(19) United States
(12) Patent Application Publication
(10) Pub. No.: US 2025/0255808 A1
(43) Pub. Date: Aug. 14, 2025

(54) NANOGEL PLATFORM TECHNOLOGY FOR LONG-TERM BIOLOGICS THERAPY

(71) Applicant: UNIVERSITY OF MARYLAND, BALTIMORE, Baltimore, MD (US)

(72) Inventor: Tao LOWE, Hummelstown, PA (US)

(21) Appl. No.: 18/859,241

(22) PCT Filed: Apr. 26, 2023
(86) PCT No.: PCT/US2023/020081

§ 371 Date: Oct. 23, 2024

Related U.S. Application Data
(60) Provisional application No. 63/334,938, filed on Apr. 26, 2022.
(60) Provisional application No. 63/334,896, filed on Apr. 26, 2022.

(51) Int. Cl. A61K 9/06; A61K 31/7105; A61K 47/36

(52) U.S. Cl. CPC A61K 9/06; A61K 31/7105; A61K 47/36

(57) ABSTRACT

Biologics, including peptides, proteins, antibodies, nucleic acids (DNA and RNA), oligonucleotides, vaccines, or complex combinations of these substances, are important for treating various types of diseases and tissue and organ regeneration. However, biologics are not stable and have short half-lives, making effective delivery to patients difficult. Therefore, there is an unmet need in the art to increase the stability and half-lives of biologics for long-term bioavailability, therapy, treatment and repair. This invention provides a nanogel platform technology that can load biologics in aqueous solution with high loading efficiency without using any organic solvent and also sustain the release of active biologics in the body for more than 2 months.

### CROSS-REFERENCE TO RELATED APPLICATIONS

This application is a 371 National Stage application of PCT Application No. PCT/US2023/020081, filed 26 Apr. 2023, which claims the benefit of U.S. provisional application Ser. No. 63/334,938, filed 26 Apr. 2022 and U.S. provisional application Ser. No. 63/334,896, filed 26 Apr. 2022. The entire contents of these applications are hereby incorporated by reference as if fully set forth herein.

### GOVERNMENT FUNDING SUPPORT

This invention was made with government support under grant no. EY023853, awarded by the National Institutes of Health. The government has certain rights in the invention.

### BACKGROUND

### 1. Field of the Invention

The invention relates to the general field of medicine, and in particular to a nanogel formulation for biologics which provides long-term sustained release of the biologic to the subject in need of such treatment.

### 2. Background of the Invention

Biologics, including peptides, proteins, antibodies, nucleic acids (DNA and RNA), oligonucleotides, vaccines, or complex combinations of these substances, are important in treating all types of diseases and in the regeneration of tissues and organs. Such treatments and regenerations are used for various conditions, not limited to neurological, eye (including retina), brain, ear, temporomandibular, dental, oral, facial, blood, bone, cartilage, joint, heart and vascular system, lung, bronchus, skin, muscle, reproductive, liver, cancer, diabetes, pancreas, gastrointestinal tract, endocrine tissues or glands, kidney, breast, oral, head, neck, esophageal, thyroid, fat, muscle, gastrointestinal stromal, intrahepatic bile duct, bladder, colon, rectum, vaginal, prostate, testicular, pancreas, cervix, uterine, pleura, immune system, and the like, or any disease or condition that would benefit from long-term sustained release of an active agent for treatment. For example, diseases and conditions such as type 1 diabetes, type 2 diabetes, diabetic retinopathy, fungal, seizure, stroke, depression, hepatitis C, age-related macular degeneration, glaucoma, dry eye, Alzheimer's disease, Parkinson's disease, neurological disorders, pain, temporomandibular joint disorders, opioid overdose, rheumatoid arthritis, and the like; and also including injuries, fractures and/or other damage to tissues and organs.

Biologics are not stable, however, and have short half-lives from a few minutes to about one month. Currently methods used for sustained release of biologics include hydrogels, implants, pumps, microparticles and nanoparticles. Hydrogels and implants require surgical implantation and removal (if the biomaterials are not biodegradable), which is costly and invasive. Pumps have the disadvantage of clunkiness and carry the burden of requiring extra batteries or a charger, potential battery failure, and infection risk.

Microparticles and nanoparticles include polymeric micro/nanoparticles made of poly(lactic acid-co-glycolic acid) (PLGA), poly(lactic acid) (PLA), poly(ethyleneglycol)-poly(lactide), polyalkylcyanoacrylate, polyamines, poly(ethylenimine), poly(amidoamine), poly(propylene imine) or albumin, liposomes, dendrimers, polymeric micelles, inorganic and carbon nanotubes. These microparticles/nanoparticles usually do not achieve more than one month of useful life, sometimes need to use organic solvents for biologic encapsulation, which can denature the biologics, have toxicity and/or high initial burst issues, and/or do not or hardly cross biological barriers. In addition, they are usually loaded with biologics by adsorption, diffusion and covalent attachment methods, which have low drug loading efficiency causing significant waste of biologics which are usually very expensive. The covalent attachment of biologics has a drawback of causing loss of drug activity.

### SUMMARY OF THE INVENTION

Therefore, there is an unmet need in the art for a product and method that can increase the loading efficiency, usage, stability, and half-lives of biologics for long-term bioavailability by providing long-term sustained release of biologics for therapy, treatment and repair in patients. In particular embodiments, the present invention relates to 1. A nanogel pharmaceutical composition, comprising: (a) a biologic medicament; and (b) a biodegradable nanogel composition comprising (i) a macromer according to Formula I wherein CLU is a crosslinkable unit, HS is a hydrolysable spacer, and DU is a dextran or polysaccharide unit (CLU-HS-DU Formula I); (ii) a hydrolyzable crosslinker; (iii) a monomer; and (iv) an initiator, wherein the macromer, the monomer, and the hydrolysable crosslinker are reacted with an initiator to form a biodegradable nanogel.

In certain preferred embodiments, the biologic medicament is selected from the group consisting of a protein, a peptide, a nucleic acid, an antibody, a monoclonal antibody, a gene, a nucleic acid (RNA or DNA), a nucleotide, a oligonucleotide, a siRNA, a mRNA, an aptamer, a vaccine, a receptor, an enzyme, a ligand, a hormone, a blood product, a biopolymer, a natural polymer, a biomolecule, a biomacromolecule, a poly(amino acid), a protein kinase, a cytokine, a growth factor, a differentiation factor, a neurotrophic factor, a stem cell factor, a fusion protein, a carbohydrate, a polysaccharide, a lipid, a lipopolysaccharide, a glycosaminoglycan, a steroid, a nutrient, a tumor necrosis factor (TNF) inhibitor, an interleukin (IL) inhibitor, a B-cell inhibitor, or a T-cell inhibitor, or a combination thereof. Most preferably, biologic medicament is selected from the group consisting of a peptide hormone, an antibody, an aptamer, and an siRNA or a combination thereof.

In some embodiments, CLU is a hydrolytically or enzymatically degradable, crosslinked nanogel. In some embodiments, the initiator is 2-hydroxy-4′-(2-hydroxyethoxy)-2-methylpropiophenone. In some embodiments, the monomer is N-isopropylacrylamide.

In certain embodiments, the biologic medicament is released from the nanogel for at least 20 days, or at least 30 days, or at least 60 days, or at least 90 days.

In preferred embodiments, the biologic medicament is loaded in the nanogels in aqueous solution with at least 50% loading efficiency or at least 80% loading efficiency, or at least 95% loading efficiency.

In certain embodiments, the invention relates to a method of treating a subject in need of a biologic medicament, comprising administering to the subject a pharmaceutical composition as described herein.

### BRIEF SUMMARY OF THE DRAWINGS

Certain embodiments are illustrated by way of example, and not by way of limitation, in the figures of the accompanying drawings.

FIG. 1 is a schematic drawing showing the synthetic method for nanogels.

FIG. 2A and FIG. 2B present LC-MS/MS spectra of 100 ng/mL human insulin showing the effects of column type on insulin peak sharpness and intensity.

FIG. 3A and FIG. 3B are MS spectra showing that increasing the mobile phase from 15%-40% to 15% to 70% improved the sharpness and the strength of the HPLC peaks. FIG. 3C and FIG. 3D present MS spectra that show both peak sharpness and strength significantly increased when the percentage of acetic acid in the mobile phase increased from 0.2 to 1%. Phase A=water; phase B=acetonitrile.

FIG. 4A, FIG. 4B, and FIG. 4C are LC-MS/MS spectra showing the effect of THF as a mobile phase modifier on insulin peak sharpness or strength (FIG. 4A: 0% THF; FIG. 4B: 5% THF; FIG. 4C: 10% THF).

FIG. 5A and FIG. 5B are LC-MS/MS spectrum of rat plasma and rat plasma spiked with human insulin, respectively.

FIG. 6A through FIG. 6P present LC-MS/MS spectra of insulin extracted from different eye tissues obtained with and without solid phase extraction. FIG. 6A: AH—before SPE; FIG. 6B: SPE—after SPE; FIG. 6C: VH—before SPE; FIG. 6D: VH—after SPE; FIG. 6E: Retina—before SPE; FIG. 6F: Retina—after SPE; FIG. 6G: I/CB—before SPE; FIG. 6H: I/CB—after SPE; FIG. 6I: Choroid—before SPE; FIG. 6J: Choroid—after SPE; FIG. 6K: Cornea—before SPE; FIG. 6L: Cornea—after SPE; FIG. 6M: Lens—before SPE; FIG. 6N: Lens—after SPE; FIG. 6O: Sclera—before SPE; FIG. 6P: Sclera—after SPE.

FIG. 7A, FIG. 7B and FIG. 7C are graphs showing significant nonspecific binding by MS spectra to the surfaces of glass and plastic inserts, resulting in about 100-fold reduction in insulin peak intensity when compared to glass vials.

FIG. 7D, FIG. 7E and FIG. 7F are graphs showing the effect of using 0.1% BSA (FIG. 7D) or coating both plastic and glass vials with 2% BSA overnight prior to their use (FIG. 7E and FIG. 7F) by MS spectra.

The extent of insulin adsorption increased with time and it was irreversible, as showing in the MS spectra of FIG. 7G and FIG. 7I, respectively. Insulin adsorption to the plastic inserts was transient and the peak intensity increased considerably, as shown in FIG. 7H.

FIG. 8 is a graph showing the effect of monomer to macromer ratio (see numbered nanogels) on insulin release.

FIG. 9 is a graph showing the effect of initiator concentration on insulin release kinetics of nanogels made of Dex-PLA-HEMA macromer with different DP and DS.

FIG. 10A and FIG. 10B present data on the effect of degree of substitution (DS) on insulin release.

FIG. 11 presents data on the effect of degree of polymerization (DP) on insulin release.

FIG. 12 is a graph comparing the effect of the type of macromer on insulin release as indicated.

FIG. 13 is a graph showing the effect of negatively charged acrylic acid and zinc in the nanogel on insulin release. The error bar stands for standard error.

FIG. 14 is a graph showing the effect of positively charged 2-aminoethyl methacrylate in the nanogel on insulin release. The error bar stands for standard error.

FIG. 15A, FIG. 15B, FIG. 15C, FIG. 15D, and FIG. 15E are graphs showing the effect of the monomer to macromer ratio on the cytotoxicity of nanogels made of NIPAAM, Dex-PLA-HEMA and acrylic acid to ARPE-19 cells.

FIG. 16A is a graph showing effects of the charge type (negative and positive charges from acrylic acid (A) and 2 amino ethyl methacrylate (B), respectively) of nanogels on the cytotoxicity of the nanogels to ARPE-19 cells.

FIG. 16B is a graph showing the effects of the PLA and PCL in the Dex-PCL-HEMA macromer and the charge type (negative and positive charges from acrylic acid (A) and 2 amino ethyl methacrylate (B), respectively) on the cytotoxicity of the nanogels to ARPE-19 cells at different concentrations.

FIG. 17 is a bar graph showing results for permeability of nanogels across the ARPE-19 cell membrane at 37° C.

FIG. 18A and FIG. 18B are bar graphs showing results for permeability of nanogels across the piglet sclera at 37° C., and the piglet cornea at 32° C., respectively.

FIG. 19 is a bar graph showing fluorescent intensities of nanogels in different ocular tissues obtained after 1 and 7 days of subconjunctival injection in the rat eye. The values are normalized with the florescent ratio of the control group and values greater than 1 indicate nanogel distribution into the eye.

FIG. 20 is a bar graph showing the amount of human insulin recovered in the different eye tissues of SD rats 1 day after subconjunctival injections of insulin and insulin loaded nanogel (NG).

FIG. 21 is a schematic showing treatment methods for macular degeneration using Bevacizumab nanogels.

FIG. 22 is a drawing showing the method used for producing Bevacizumab-loaded nanogels.

FIG. 23 presents attenuated total reflectance-Fourier transform infrared (ATR-FTIR) spectra of nanogels containing 0 mol % acrylic Acid (AA) with respect to NIPAAm and 0 wt % Avastin loaded in the nanogel (curve A), 0 mol % AA, 12.5 wt % Avastin (curve B), 2 mol % AA, 0 wt % Avastin (curve C), 2 mol % AA, 12.5 wt % Avastin (curve D), 5 mol % AA, 0 wt % Avastin (curve E), 5 mol % AA, 12.5 wt % Avastin (curve F), and Avastin alone (curve G).

FIG. 24 is a set of photomicrographs showing representative QI mode AFM images of nanogels containing 0, 2 or 5 mol % acrylic acid (AA) as indicated, loaded with and without 12.5 wt % Avastin in water at 37° C.

FIG. 25A and FIG. 25B show the cumulative release and daily release dose of Avastin, respectively from nanogels containing 2 or 5 mol % acrylic acid (AA) loaded with 37.5 wt % Avastin® in PBS (pH 7.4) as a function of time at 37° C. FIG. 25C and FIG. 25D show the cumulative release and daily release dose of Avastin, respectively from nanogels containing 5 mol % acrylic acid (AA) loaded with 12.5 or 37.5 wt % Avastin® in PBS (pH 7.4) as a function of time at 37° C. The nanogels can sustain the release of Avastin for 100 days (n=3).

FIG. 26A and FIG. 26B present far-UV and fluorescence emission scans of native, denatured, and released Avastin® from the nanogels, respectively.

FIG. 27 is a bar graph showing the cytotoxicity of the nanogels to fetal human retinal pigment epithelial (fhRPE) cells at nanogel concentrations of 0.1, 0.2, 0.5, 1, 2, 5, and 10 mg/mL, assessed using an MTT assay (n=4) after 72 hours.

FIG. 28 presents data on VEGF expression of hfRPE cells treated with and without UV. HfRPE cells were exposed to UV for 1 h, and then treated with Avastin at 0.25 mg/mL, and nanogels containing 2 mol % AA with and without 12.5 wt % Avastin at 2 mg/mL. Cells with and without UV treatment were used as controls. n=3.

FIG. 29A and FIG. 29B show the cumulative release and daily release dose of siRNA, respectively from nanogels in PBS (pH 7.4) as a function of time at 37° C.

FIG. 30A and FIG. 30B show the cumulative release and daily release dose of anti-TNFα, respectively, from nanogels in PBS (pH 7.4) as a function of time at 37° C.

### DETAILED DESCRIPTION OF EMBODIMENTS OF THE INVENTION

### 1. Overview

This invention provides a nanogel platform technology and method that can be used to load one or more biologics with more than 98% loading efficiency in aqueous solution without using any organic solvent and also to provide a sustained release of the biologic for more than 2 months. The product and method are useful for any biologic, such as proteins and peptides, nucleic acids, and the like, including insulin, antibodies such as bevacizumab (Avastin®) or anti-TNF-α antibodies, siRNAs, and the like for the treatment of such conditions as age-related macular degeneration, diabetic retinopathy and cancer.

### 2. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

As used herein, the term “about” means plus or minus 20 percent of the recited value, so that, for example, “about 0.125” means 0.125±0.025, and “about 1.0” means 1.0±0.2. Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

As used herein, the term “biologic” refers to non-small molecule pharmaceuticals for treatment of a disease or condition. The term includes, but is not limited to peptides, proteins, antibodies, monoclonal antibody (mAb) products, genes, nucleic acids (RNA or DNA), nucleotides, oligonucleotides, siRNA, mRNA, aptamers, vaccines, receptors, enzymes, ligands, hormones, gene and cellular therapies, blood products, biopolymers, natural polymers, biomolecules, biomacromolecules, amino acids, protein kinases, cytokines, growth factors, differentiation factors, neurotrophic factors, stem cell factors, fusion proteins, carbohydrates, polysaccharides, lipids, lipopolysaccharides, glycosaminoglycans (GAGs), steroids, insulin, insulin like growth factor 1, insulin like growth factor 2, insulin-like growth factor-binding protein 3, tumor necrosis factor (TNF), anti-TNFα, tumor necrosis factor-binding protein (TNF-bp), nerve growth factor (NGF), brain derived neurotrophic factor (BDNF), glial derived neurotrophic factor (GDNF), neurotrophic growth factor, neurotrophic factor 3 (NT3), bone growth factor, osteoprotegerin (OPG), bone morphogenetic protein 2 (BMP2), bone-derived growth factor, platelet-derived growth factor (PDGF), interleukin, interleukin-1 (to 18) receptor antagonist (IL-Ira), interferon (alpha, beta, gamma), consensus interferon, erythropoietin, granulocyte-colony stimulating factor (GCSF), leptin (OB protein), heparin, fibroblast growth factor (FGF), transforming growth factor (TGF), platelet transforming growth factor, milk growth factor, endothelial growth factors (EGF), endothelial cell-derived growth factors (ECDGF), alpha-endothelial growth factors, beta-endothelial growth factor, vascular endothelial growth factor (VEGF), hepatocyte stimulating factor, plasmacytoma growth factor, 4-1 BB receptor (4-1BBR), TRAIL (TNF-related apoptosis inducing ligand), artemin (GFRalpha3-RET ligand), BCA-1 (B cell-attracting chemokine1), B lymphocyte chemoattractant (BLC), B cell maturation protein (BCMA), keratinocyte growth factor (KGF), thrombopoietin, megakaryocyte derived growth factor (MGDF), keratinocyte growth factor (KGF), BRAK, C-10, Cardiotrophin 1 (CT1), CCR8, thyroid stimulating hormone (TSH), sex hormone binding globulin (SHBG), prolactin, luteotropic hormone (LTH), lactogenic hormone, parathyroid hormone (PTH), melanin concentrating hormone (MCH), luteinizing hormone (LHb), growth hormone (HGH), follicle stimulating hormone (FSHb), amphotericin B, Alzheimer vaccine, nutrients, heparin, protein A, streptavidin, beta-galactosidase, beta-amyloid, eukaryotic initiation factor-4G, granulocyte macrophage colony stimulating factor (GM-CSF), novel erythropoiesis stimulating protein (NESP), thrombopoietin, tissue plasminogen activator (TPA), urokinase, streptokinase, kallikrein, tumor necrosis factor (TNF) inhibitors, interleukin (IL) inhibitors, B-cells inhibitors, T-cells inhibitors, collagen, gelatin, elastin, elastin-like-peptides, fibrin, silk, dextran, hyaluronic acid, celluloses, chitosan, alginate (alginic acid), and the like, and any combination thereof.

As used herein, the terms “subject,” “individual,” “host,” and “patient,” are used interchangeably to refer to any animal, and can include humans, simians, avians, felines, canines, equines, rodents, bovines, porcines, ovines, caprines, mammalian farm animals, mammalian sport animals, and mammalian pets. A “subject in need” refers to a subject suffering from or likely to be suffering from a disease or condition that can be benefitted by administration of a biologic medicament.

As used herein, the term “macromer” refers to structures according to Formula I:

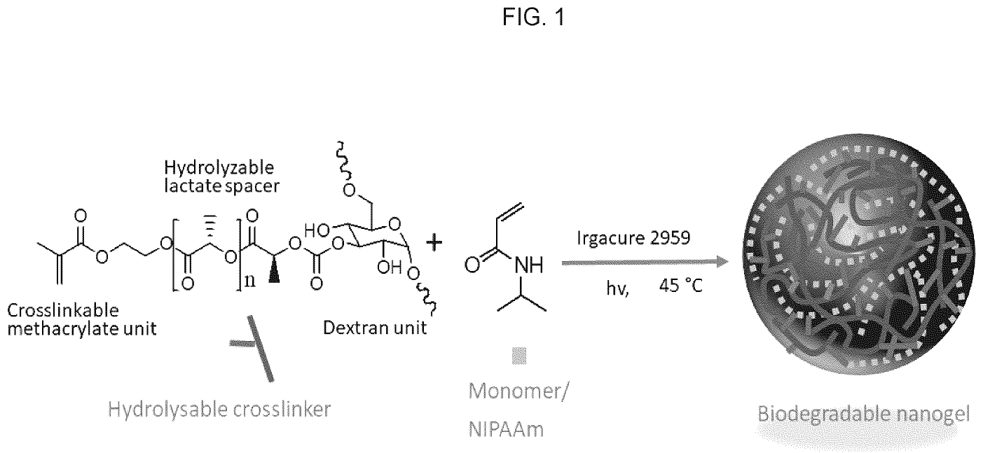

CLU-HS-DU,

where CLU is a crosslinkable unit, HS is a hydrolyzable spacer, and DU is a dextran or polysaccharide unit. Preferably, the macromer is an oligolactate-(2-hydroxyethyl methacrylate) (PLA-HEMA) grafted dextran molecules (Dex-PLA-HEMA) or a poly-ε-caprolactone-(2-hydroxyethyl methacrylate (Dex-PCL-HEMA) grafted dextrone molecules used in the production of the nanogels. See FIG. 1, which illustrates the nanogel general synthesis process.

As used herein, the term “monomer” refers to an N-isopropylacrylamide (NiPAAm), N-alkylacrylamide, N-n-propylacrylamide, N-isopropylmethacrylamide, or a derivative.

As used herein, the term “degree of polymerization (DP)” refers to the average number of units on a polymer molecule, for example the number of lactide units on the PLA-HEMA segment of a macromer.

As used herein, the term “degree of substitution (DS) refers to the average number of units per length of macromer, for example the number of PLA-HEMA units per 100 glucose units of dextran.

As used herein, the term “nanoparticle” refers to particles that have a size of about 1 nm to 1000 nm.

As used herein, the term “nanogel” refers to crosslinked particles that have a size of about 1 nm to 1000 nm, preferably about 1 to 600 nm, and most preferably about 10 to about 350 nm.

### 3. Embodiments of the Invention

### A. Introduction

The nanogel compositions according to this invention are composed of crosslinkable methacrylate units with a spacer and a dextran unit (see FIG. 1) which are crosslinked with a monomer such as NiPAAm using a hydrolyzable crosslinker such as Irgacure® 2959 to form a biodegradable nanogel structure. See FIG. 1.

### B. Nanogel Components

The hydrolyzable and crosslinkable macromer units useful in the invention comprise two or more acrylate unit, preferably 2-hydroxyethyl methacrylate, linked to a hydrolyzable spacer or spacers grafted to a macromolecule unit. The hydrolyzable spacer or spaces include oligolactate or poly-ε-caprolactone. The macromolecule include polysaccharide, preferably dextran, hyaluronic acid, celluloses, chitosan, alginate (alginic acid), collagen, gelatin, elastin, elastin-like-peptides, proteins, peptides, antibodies, DNA, siRNA, RNA, mRNA, genes, enzymes, nucleic acids, nucleotides, receptors, growth factors, differentiation factors, neurotrophic factors, stem cell factors, vaccines, fibrins, glycosaminoglycans (GAGs), silk, bioploymer, natural polymer, polylactic-co-glycolic acid, polyethylene glycol, poly(ethylene oxide)-poly(propylene oxide)-poly(ethylene oxide), poly(ethylene oxide)-co-poly(L-lactic acid), biotinylated poly(ethylene glycol-block-lactic acid), pluronic acid, polaxamer, polyester, polyamide, poly(amino acid), polyurethane, polyorthoester, polyanhydride, polyethylene terephthalate, polycarbonate, polyfumarate, polycyanoacrylate, poly(alkylcyanoacrylate), polyphosphazene, polyphosphoester, or poly(bis(p-carboxyphenoxy) propane-sebacic acid). The purpose of these units in the formulation of nanogels according to the invention is to stabilize the nanogels in water, add crosslinked structure to the nanogels to slow the biologic release, and modulate the biologic release kinetics by adjusting the hydrolyzable spacer length and number and thus degradable rate of the nanogels.

Monomer units useful in the invention include NiPAAm, N-alkylacrylamide, N-n-propylacrylamide, N-isopropylmethacrylamide, or a derivative, or a mixture thereof. The purpose of these units in the formulation of nanogels according to the invention is that the monomer is soluble in aqueous solution, and its polymer is thermoresponsive and hydrophobic at body temperature to have high biologic loading efficiency and low initial burst and modulate the biologic release kinetics. Therefore, any suitable monomer can be used.

The reaction forming the nanogel is initiated by a suitable initiator such as Irgacure® 2959 (2-hydroxy-4′-(2-hydroxyethyl)-2-methyl propiophenone), 2,2′-azobis[2-(2-imidazolin-2-yl)propane]dihydrochloride, 1-hydroxycyclohexylphenyl-ketone, 2-hydroxy-2-methyl-1-phenylpropanone, 2,2-dimethoxy-1,2-diphenyl-ethan-1-one, 2-(4-Methylbenzyl)-2-(dimethylamino)-1-(4-morpholinophenyl)butan-1-one, alpha hydroxy ketones, phosphine oxides, benzophenone, thioxanthones, 2,2-dimethoxy-2-phenylacetophenone, isopropyl thioxanthone, 2-ethylhexyl-(4-N,N-dimethyl amino)benzoate, ethyl-4-(dimethylamino)benzoate, peroxides, benzoyl peroxide, molecular oxygen, azobisisobutyronitrile, camphorquinone, eosin Y, triethanolamine, 1-vinyl-2-pyrrolidinone, and the like, or a mixture thereof. The preferred initiator is Irgacure® 2959, which is a non-yellowing radical photoinitiator for the UV curing of systems. Any similar initiator can be used as is known in the art. The reaction is initiated with UV light of a suitable wavelength, depending on the initiator used. For Irgacure® 2959, light of about 365 nm is recommended for the reaction.

Additional components that can be included in the nanogels of this invention include components that modify the bulk and/or surface properties of the nanogel. Comonomer acrylic acid, 2 amino-ethyl methacrylate, 2-ally meta-cresol, monomers containing carbolic acid side group, monomers containing primary or secondary amine side group, or a combination thereof, for example, can be added during synthesis in order to modify the surface and bulk properties of the nanogels, modulate biologic release kinetics and amount from the nanogels, enhance the stability of biologics (use of 2-ally meta-cresol), and/or increase cellular uptake and/or tissue penetration of the nanogels and/or biologics. Zinc salts, such as zinc sulfate, polyhydric alcohols, glycerol, sucrose, glucose, or trehalose, and the like, or a combination thereof, can be used as an additive to slow release of the biologic medicament from the nanogel. For example, zinc sulfate can be added during synthesis of the nanogel in a range of about 0.1 wt % to about 500 wt %, preferably about 1 wt % to about 500 wt %, and most preferably about 1 wt % to about 250 wt %.

### C. Synthetic Methods

The components of the nanogel, including the macromer (i.e. Dex PCL-HEMA macromer or Dex-PLA-HEMA macromer), NiPAAm or another monomer, acrylic acid or other modifier (optional), the biologic medicament or medicaments, Igracure® or another initiator, are mixed together in water. Initiator is added and the mixture is subjected to UV irradiation at 365 nm wavelength (for Igracure®) and 1 W/cm² intensity for about 10-60 minutes under stirring at 200-300 rpm at about 45° C. See FIG. 1. The product then is subjected to dialysis for 5 hours against water, using a 50-300 kDa dialysis membrane, with frequent change of the dialysis water (every 0.5 h to 1 h) followed by lyophilization.

### D. Modification of Nanogel Formulations

Comonomer acrylic acid, 2 amino-ethyl methacrylate, 2-ally meta-cresol, monomers containing carbolic acid side group, or a monomer containing primary or secondary amine side group, or a combination thereof is added together with the monomer and macromer before the UV-emulsion polymerization is initiated. The amount of these comonomers to be added is at molar ratio to the monomer at about 0.1 mol % to about 50 mol %, preferably about 1 mol % to about 30 mol %, and most preferably about 1 mol % to about 25 mol %. Zinc salts, such as zinc sulfate, polyhydric alcohols, glycerol, sucrose, glucose, or trehalose, and the like, or a combination thereof is added before the initiator is added in a range of about 0.1 wt % to about 500 wt %, preferably about 1 wt % to about 500 wt %, and most preferably about 1 wt % to about 250 wt %.

Nanogels synthesized with different macromers and/or different DP/DS values for the macromer are expected to behave differently and may exhibit different drug release profiles. Dex-PCL (poly-ε-caprolactone)-HEMA macromers versus macromers containing PLA produce different effects, which can be useful in modifying the release profile of the medicament to the patient. PCL is less polar and exhibit a slower degradation kinetics than PLA and may better control drug release for long term use.

Generally, the actual DP value was greater than the theoretical DP value in a particular nanogel and the actual DS value was less than the theoretical DS value. As expected, however, the longer the reaction time, the higher the DS value and the yield. The DS values of PCL-HEMA were relatively less than the DS values of the corresponding PLA-HEMA oligomers showing that PCL is less reactive towards dextran than PLA. See the examples below for effects on the nanogels.

Nanogel synthesis yield and particle size increased considerably with increasing initiator concentration, particularly up to 1.0% and 1.5% of the initiator concentration for yield and particle size, respectively. The morphology, mechanical strength and stability of the nanogel product can be adjusted as follows: change chemical composition, adjust the relative amount of the components including the macromer, monomer and modifiers, and change the DP and DS of the macromer.

### E. Loading of Nanogel Formulations

To load the nanogel, the biologic or biologics to be loaded are included in the synthetic mixture. Generally, a concentration of about 1×10⁻³ wt % to about 50 wt %, preferably about 1×10⁻³ wt % to about 50 wt %, and most preferably about 1×10⁻³ wt % to about 35 wt % can be used. The amount can easily be determined by the practitioner depending on the nature of the biologic(s), the dose required for a particular patient and disease or condition from which the patient suffers. See synthetic method, above. Quantitation of the loaded medicament in the nanogel can be determined using an ultra performance liquid chromatography (UPLC), mass spectroscopy, ELISA, and/or bicinchoninic acid assay (BCA) method. See Example 1, below.

The percentage synthesis yield is calculated as (the amount of nanogels obtained after lyophilization/theoretical weight of the nanogel×100). Percent biologic loading is calculated as (total amount of biologic loaded−total amount of biologic recovered in the dialysis media)/total amount of biologic loaded×100). Release from the nanogels can be measured as shown in Examples, below.

### F. Toxicity

The toxicity of the nanogels and their degradation product was investigated by MTT assay, as described herein. The nanogels exhibited very low cytotoxicity in all tests with cell viability more than 90% at concentration up to 2 mg/mL except for the nanogels made of PLA-containing macromer and positively charged 2-aminoethyl methacrylate. The nanogels made of PCL-containing macromer and negatively charged acrylic acid are not cytotoxic at concentration up to 10 mg/mL.

### G. Biologics

The biologics that can be loaded into the nanogels of the invention for release to a patient include any protein, peptide, nucleic acid, or the like as known in the art. Preferably, it is contemplated that the nanogel invention is useful for administration to a patient of antibody medications, peptide hormones, RNA and DNA medications, and the like as are known to the person skilled in the art. Preferred examples of biologics are monoclonal antibodies, growth factors, aptamers, peptide or protein hormones, nucleic acids (RNA or DNA), nucleotides, oligonucleotides, siRNA, mRNA, gene therapies, vaccine proteins and peptides, receptors, enzymes, ligands, hormones, gene and cellular therapies, blood products, biopolymers, natural polymers, biomolecules, biomacromolecules, polysaccharides, lipids, lipopolysaccharides, glycosaminoglycans (GAGs), steroids, nutrients, amino acids, protein kinases, cytokines, growth factors, differentiation factors, neurotrophic factors, stem cell factors, fusion proteins, carbohydrates, TNF inhibitors, interleukin inhibitors, B cell inhibitors, T cell inhibitors, and the like. Specific, non-limiting examples of suitable biologics are Humalog® (insulin lispro), Admelog® (lispro), Novolog® (aspart), Fiasp® (aspart), Apidra® (gluisine), Humulin® R (U-100) (regular human insulin (RHI)), Novolin® R (regular human insulin (RHI)), Humulin® R (U-500) (regular human insulin (RHI)), Humulin® N (neutral protamine Hagedorn (NPH), Novolin® N (neutral protamine Hagedorn (NPH), Lantus® (glargine (100 U/ml)), Basaglar® (glargine (100 U/ml)), Levemir® (detemir), Toujeo® (glargine (300 U/ml)), Tresiba® (degludec), Humulin® 70/30 (70% neutral protamine hagedorn and 30% regular human insulin), Novolin® 70/30 (70% neutral protamine hagedorn and 30% regular human insulin), HumaLog® Mix 75/25 (75% insulin lispro protamine and 25% lispro), HumaLog® Mix 50/50 (50% insulin lispro protamine and 50% lispro), NovoLog® Mix 70/30 (70% insulin aspart protamine and 30% aspart), Ryzodeg® 70/30 (70% insulin degludec protamine and 30% aspart), Humira^{T}M (adalimumab), Herceptin™ (trastuzumab), Avastin™ (bevacizumab), Alymsys (bevacizumab), Mvasi (bevacizumab-awwb), Vegzelma (bevacizumab-adcd; CT-P16), Zirabev (bevacizumab), Lucentis® (ranibizumab), BYOOVIZ™ (ranibizumab-nuna), EYLEA® (aflibercept), Botox™ (onabotulinumtoxinA), insulin like growth factor 1, insulin like growth factor 2, insulin-like growth factor-binding protein 3, TNF, anti-TNFα, TNF-bp, nerve growth factor (NGF), brain derived neurotrophic factor (BDNF), glial derived neurotrophic factor (GDNF), neurotrophic growth factor, neurotrophic factor 3 (NT3), bone growth factor, osteoprotegerin (OPG), bone morphogenetic protein 2 (BMP2), bone-derived growth factor, platelet-derived growth factor (PDGF), interleukin, interleukin-1 (to 18) receptor antagonist (IL-Ira), interferon (alpha, beta, gamma), consensus interferon, erythropoietin, granulocyte-colony stimulating factor (GCSF), leptin (OB protein), heparin, fibroblast growth factor (FGF), transforming growth factor (TGF), platelet transforming growth factor, milk growth factor, endothelial growth factors (EGF), endothelial cell-derived growth factors (ECDGF), alpha-endothelial growth factors, beta-endothelial growth factor, vascular endothelial growth factor (VEGF), hepatocyte stimulating factor, plasmacytoma growth factor, 4-1 BB receptor (4-1BBR), TRAIL (TNF-related apoptosis inducing ligand), artemin (GFRalpha3-RET ligand), BCA-1 (B cell-attracting chemokinel), B lymphocyte chemoattractant (BLC), B cell maturation protein (BCMA), keratinocyte growth factor (KGF), thrombopoietin, megakaryocyte derived growth factor (MGDF), keratinocyte growth factor (KGF), BRAK, C-10, Cardiotrophin 1 (CT1), CCR8, thyroid stimulating hormone (TSH), sex hormone binding globulin (SHBG), prolactin, luteotropic hormone (LTH), lactogenic hormone, parathyroid hormone (PTH), melanin concentrating hormone (MCH), luteinizing hormone (LHb), growth hormone (HGH), follicle stimulating hormone (FSHb), amphotericin B, Alzheimer vaccine, heparin, protein A, streptavidin, beta-galactosidase, beta-amyloid, eukaryotic initiation factor-4G, granulocyte macrophage colony stimulating factor (GM-CSF), novel erythropoiesis stimulating protein (NESP), thrombopoietin, tissue plasminogen activator (TPA), urokinase, streptokinase, kallikrein, collagen, gelatin, elastin, elastin-like-peptides, fibrin, silk, dextran, hyaluronic acid, celluloses, chitosan, alginate (alginic acid), and the like. All of these types of biologics are contemplated for use with the invention.

### H. Diseases and Conditions

Any disease or condition that can benefit from longer term administration of a biologic is suitable for treatment using the invention described herein. For example, common diseases and conditions that are treated using biologics include, but are not limited to type 1 diabetes, type 2 diabetes, diabetic retinopathy, cancer, macular degeneration, age-related macular degeneration, fungal, seizure, stroke, depression, hepatitis C, glaucoma, dry eye, Alzheimer's disease, Parkinson's disease, neurological disorders, pain, temporomandibular joint disorders, opioid overdose, rheumatoid arthritis, and the like; or any disease or condition that would benefit from long-term sustained release of an active agent for treatment; and also injuries, fractures and/or other damage to tissues and organs that need regeneration of tissues. The invention can be used in treating any tissue or organ of the body, including but not limited to neurological, eye (including retina), brain, ear, temporomandibular joint, dental, oral, facial, blood, bone, bone, cartilage, joint, heart and vascular system, lung, bronchus, skin, muscle, reproductive, liver, cancer, diabetes, pancreas, gastrointestinal tract, endocrine tissues or glands, kidney, breast, oral, head, neck, esophageal, thyroid, fat, muscle, gastrointestinal stromal, intrahepatic bile duct, bladder, colon, rectum, vaginal, prostate, testicular, pancreas, cervix, uterine, pleura, immune system, and the like.

### I. Methods of Use

The nanogels are administered to a patient in a location such that the biologic will be released near the damaged or injured tissue, or the site of action of the biologic. Preferably, a solution containing the nanogels is injected into the appropriate tissue. The solution used and the injection volume will depend on the concentration of nanogels and the location of the injection. The practitioner is able to determine an appropriate route of administration and amount.

Nanogels preferably are prepared as a suspension in a solution of water, saline, water and ethanol co-solvent, water and dimethylsulfoxide (DMSO), or the like, as determined by the practitioner, at a concentration of about 0.01 mg/mL to about 1000 mg/mL, preferably about 0.1 mg/mL to about 500 mg/mL, and most preferably about 1 mg/mL to about 250 mg/mL. A dose of the nanogel dispersion generally is given at about 1 μL to about 20 μL, and more preferably about 5 μL to about 10 μL, depending on the tissue or area of the body to be injected. Thus, an individual dose generally is about 1 mg/kg to about 800 mg/kg.

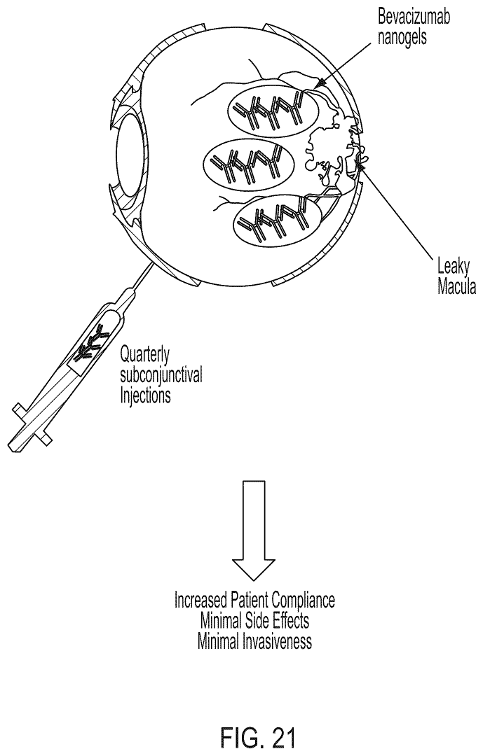

Any tissue or organ in need of treatment by the biologic can be treated according to the invention. For example, nanogels can be administered to the eye for treatment of macular degeneration. See FIG. 21. Other examples include, but are not limited to, type 1 diabetes, type 2 diabetes, diabetic retinopathy, cancer, macular degeneration, age-related macular degeneration, fungal, seizure, stroke, depression, hepatitis C, glaucoma, dry eye, Alzheimer's disease, Parkinson's disease, neurological disorders, pain, temporomandibular joint disorders, opioid overdose, rheumatoid arthritis, and the like; or any disease or condition that would benefit from long-term sustained release of an active agent for treatment; and also injuries, fractures and/or other damage to tissues and organs that need regeneration of tissues.

Doses are contemplated for administration as frequently as every two or three days, every week or two weeks, every three weeks, every month, every two months, quarterly, or semi-annually, however any dosage schedule is appropriate depending on the release rate, total amount of biologic in the administered biogels, and the condition of the patient.

### 5. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: General Methods

### A. Determination of Yield and Loading.

The yield of the prepared nanogels was calculated gravimetrically after freeze-drying. The percentage of insulin loaded was determined indirectly after measuring the amount of insulin recovered in the dialysis media. The amount of insulin in the lyophilized dialysis media was determined using an ultra performance liquid chromatography (UPLC, Waters ACQUITY UPLC®, Waters Technologies Corporation, Milford, MA, USA) method (see insulin quantitation method below). The percentage yield was calculated as (The amount of nanogels obtained after lyophilization/Theoretical weight of the nanogel×100). Percent insulin loading was calculated as (Total amount of Insulin Loaded−Total amount of Insulin recovered in the dialysis media)/Total amount of insulin loaded×100).

### B. Particle Size, Size Distribution and Zeta Potential.

The particle size, polydispersity index (PDI) and zeta potential of the freshly prepared nanocarriers were measured at a concentration of 1 mg/mL and 25 and 37° C. by using dynamic light scattering using Zetasizer Nano ZS (Malvern Instrumentsm Ltd., Westborough, MA, USA) or ALV dynamic light scattering containing ALV-CGS-8F compact goniometer system, DPSS laser (660 nm, 50 mW), and ALV-5000/EPP multiple tau digital real time correlator (ALV-Laser Vertriebsgesellschaft m.b.H., Langen, Germany). The sample dispersions were filtered through 0.45 μm syringe filters before the measurements. The nanogels were equilibrated at 37° C., for 10 minutes before starting measurement. Three replicates were performed for each measurement and reported as mean±standard deviation. Atomic Force Microscopy (AFM) with quantitative imaging mode (JPK NanoWizard 4a AFM QI Mode, Brucker, Billerica, MA, USA) was used to measure nanogel particle sizes by plating 10 μg/mL sample dissolved in water on mica substrate. Nanogels on mica were dried by nitrogen air, and when samples were rehydrated when mounted on the AFM and then equilibrated at 37° C. before being imaged. C14 probe was used, 2×2 um map imaged, and several AFM regions of interest (2-3 regions) were captured per nanogel formulation, and 100 total particles sized using ImageJ per formulation, quantities reported as mean±standard deviation.

### C. Determination of Lower Critical Solution Temperature (LCST).

The LCST of the nanoparticles was determined by measuring the size the nanogels using dynamic light scattering (DLS) over a temperature range of 15° C. to 65° C., with 1° C. intervals. Between measurements, the samples were maintained for 3 minutes at that temperature.

### D. Determination of the Thermodynamic Stability of Synthesized Nanogels.

To determine their thermodynamic stability, prepared nanogels are stored in PBS buffer at 37° C. for 4 weeks and the size of nanoparticles is measured at predetermined time intervals. The change in particle size with time is an indicator of nanoparticle stability.

### E. MTT Assays

The toxicity of the nanogels and their degradation product was investigated by MTT assay, as follows: 150 μL of Adult Retinal Pigment Epithelial (ARPE-19) cells or human fetal retinal pigment epithelial (hfRPE) cells were seeded in each well of 96-well plates at 50,000 cells/well density. After 24 hours, 30 μL solution was removed from each well and replaced by 30 μL of medium containing different concentrations of nanogels loaded with and without biologics. After 48 hours, 50 μL of 5 mg/mL MTT was added to every well and incubated at 37° C. for 4 hours. Absorbance was read at 570 nm using a BioTek cytation 5 microplate reader, for all experiments after removing all the wells content and then dissolving the formazan crystals in 150 μL of DMSO.

### F. Insulin Quantitation by UPLC.

During insulin release and stability experiments, measurements of insulin were conducted using an ultra-performance liquid chromatography (UPLC) system (Waters ACQUITY UPLC®, Waters Technologies Corp.) and a photodiode array (PDA) detector. The UPLC was fitted with a reversed phase UPLC column (ACQUITY UPLC® BEH 130 C18 Column 1.7 μm 2.1×100 mm column, Waters Technologies Corp.). A gradient solvent system comprising 0.1% formic acid and acetonitrile (0 min, 20%; 4.5 min, 40%; 5.0 min, 95%; 5.1 min, 98%; 5.5 min, 20%; 6.0 min, 20%) was used as a mobile phase. The flow rate was set at 0.4 mL/minute and the sample injection volume was 5 μL. Insulin detection was made using the photodiode array detector in the range of 190-400 nm. During the run, the column was maintained at room temperature and the autosampler temperature was set at 15° C. Insulin stock solution (1 mg/mL) was prepared in PBS for the calibration curve and standard samples were prepared by serial dilution of the stock solution (2.5, 5, 10, 15, 20, 25, 50, 75 and 100 μg/mL). For in vivo experiments, higher level of sensitivity was required and a UPLC-MS method is being developed.

### G. Insulin Quantitation by LC-MS/MS.

ELISA has been used for quantification of insulin in biological tissues. It is a highly sensitive method but lacks the ability to distinguish different insulin variants. As a result, an appropriate LC-MS/MS method was developed for the quantification of insulin extracted from biological tissues. During method development, nonspecific insulin binding to different surfaces and poor insulin fragmentation resulted in poor method sensitivity and reproducibility. Different approaches have been proposed to deal with these problems. Simple approaches include appropriate choice of stationary and mobile phases, sample preparation solvents, and purification and concentration of the extracted insulin using solid phase extraction. Other alternatives include nano HPLC, immunoenrichment (e.g. Mass Spectrometric Immunoassay Technology), reduction of insulin disulfide bonds followed by analysis of the a or b chains, or enzymatic digestion of the insulin and peptide quantitation. However, these approaches require special equipment or instruments and longer processing steps.

A SCIEX QTRAP® 5500 LC-MS system coupled with an LC-20AD XR Shimadzu HPLC system (Shimadzu USA Manufacturing Inc.), with an SIL-20 AC_{XR} auto sampler and a CTO-20 AC oven, was used for to measure low concentrations of insulin extracted from biological samples. Bovine insulin was used as internal standard. A Waters CORTECS® UPLC® C18+, 1.6 μm, 2.1×50 mm column was used as a stationary phase and a gradient solvent system comprising 1% acetic acid in water (solvent A) and of 1% acetic acid in acetonitrile (solvent B) was used as a mobile phase at a flow rate of 0.2 mL/min. The gradient system comprised 0.01 min-start; 0.5 min-20% B; 4.5 min-60% B; 7.0 min-60% B; 9.0 min-98% B; 9.5 min-98% B; 9.9 min-20% B; 11 min-stop. The column/oven temperature was set at 55° C. and the sample injection volume was set at 10 μL. During the experiment 1% acetic acid in 50% methanol was used as a washing solvent.

Quantitation was conducted in a positive ionization mode and the optimized MS conditions include, curtain gas (CUR), 20 Psi; collusion gas (CAD), 8 psi; ion spray voltage (IS), 5500 V; temperature (TEM), 600° C.; ion source gas 1 (GS1), 60 psi; Ion source gas 2 (GS2), 60 psi; Entrance potential, 10 V, vertical knob, 1.5; horizontal knob, 5.5. The M6+(968.6 to 226.3 obtained at DP=176 V, CE=53 V, CXP=14 V) and M5+(1162.3 to 226.3 obtained at DP=251.1 V, CE=63 V, CXP=14 V) fragmentation ions were monitored for human insulin and the M6+(956.1 to 226.3 obtained at DP=176 V, CE=51 V, CXP=12 V) fragmentation ion was monitored for the internal standard bovine insulin.

### H. Effects of Stationary and Mobile Phases on LS-MS/MS.

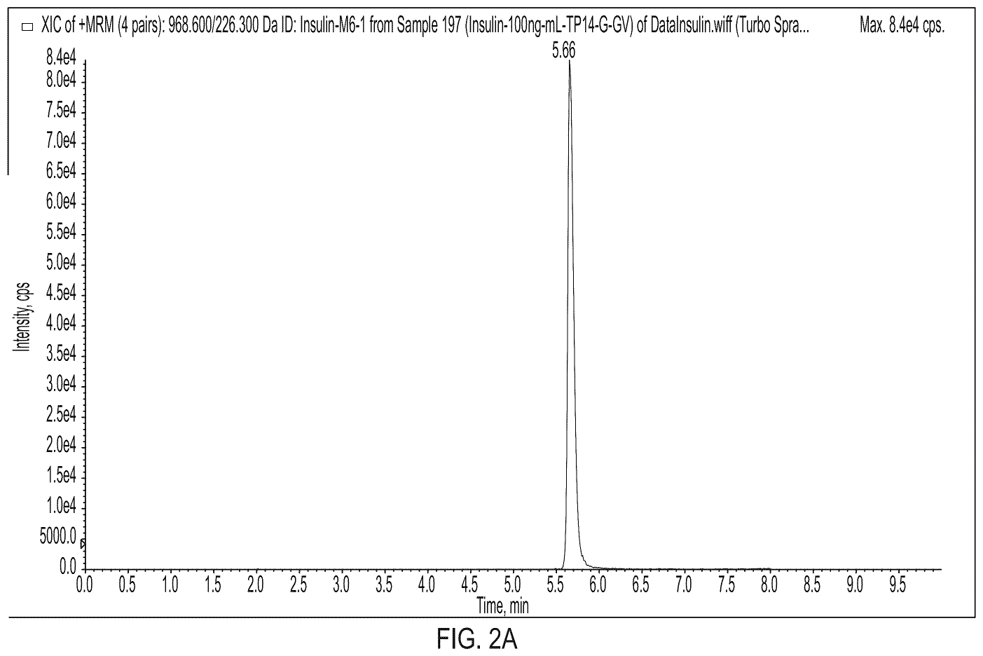

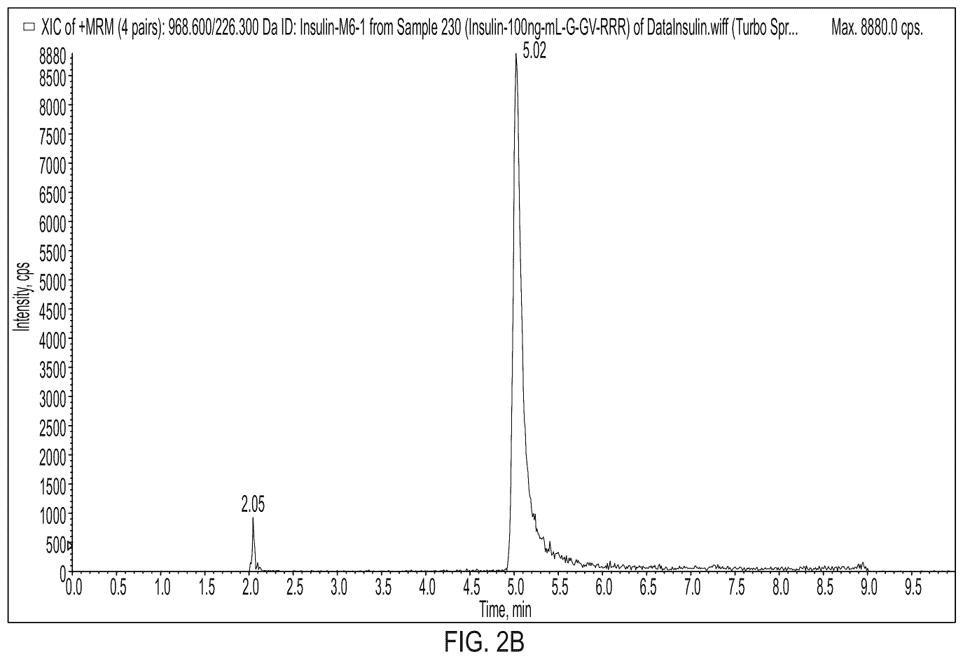

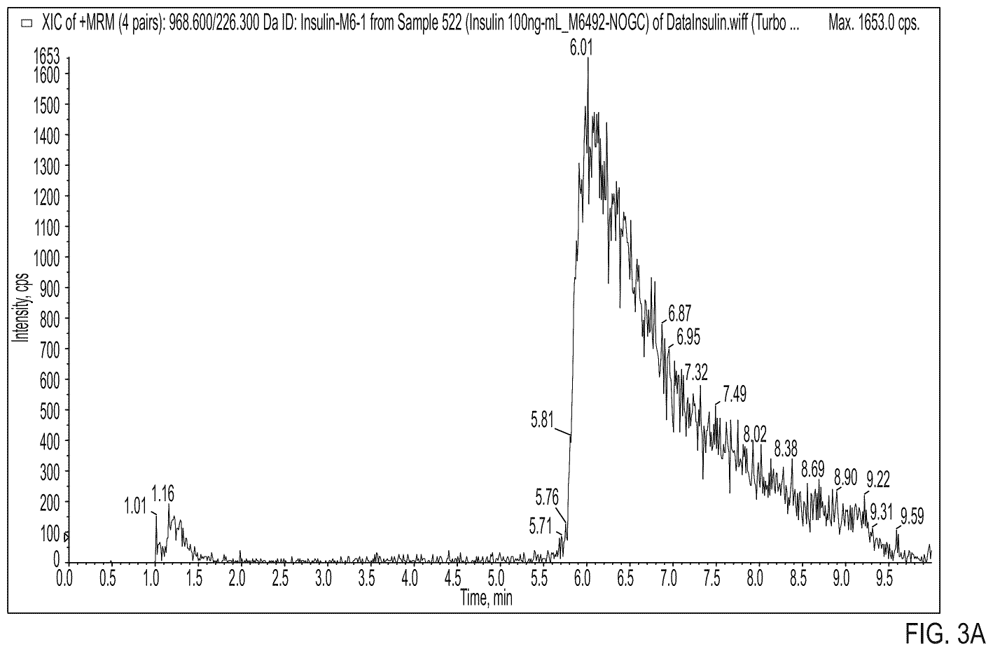

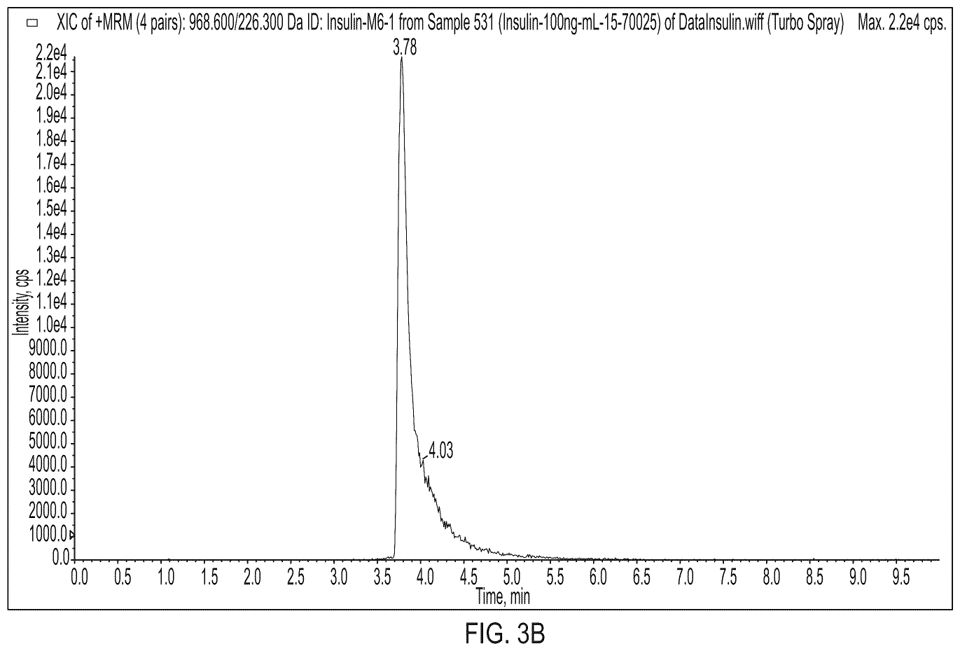

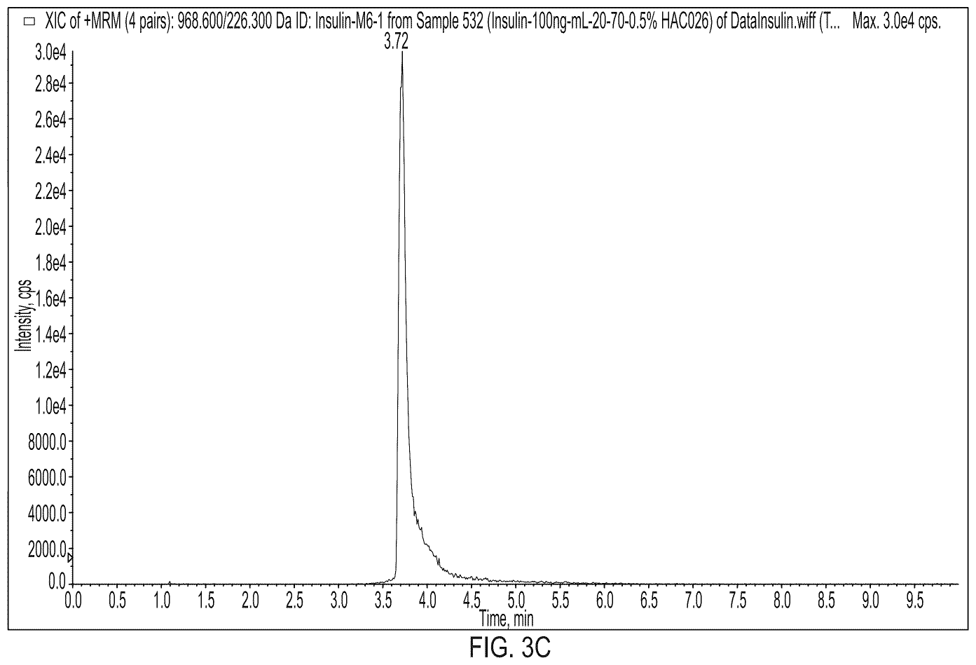

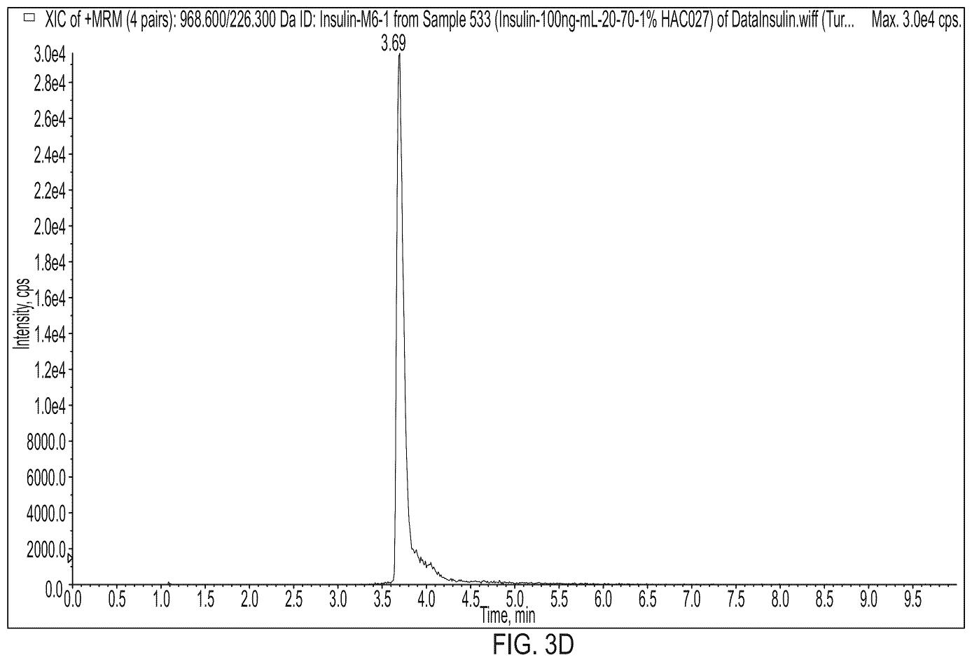

The choice appropriate stationary phase is important for the development of sensitive LC-MS technique for quantification of peptides and proteins and few specialized columns have been used. As a result, the effects of C-18 Luna and C-18 CORTECS® columns were compared and the CORTECS® column gave a sharper peak that is about 10-fold stronger than the Luna HPLC column (FIG. 2). FIG. 2A provides an MS spectrum of 100 ng/mL insulin using CORTECS® UPLC column; FIG. 2B provides an MS spectrum of 100 ng/mL insulin using a Luna HPLC column. Peak intensity=8.8×10³. Peak intensity=8.4×10⁴. Increasing the mobile phase from 15%-40% to 15% to 70% tremendously improved the sharpness and the strength of the HPLC peaks (FIGS. 3A and 3B).

In addition, both peak sharpness and strength significantly increased when the percentage of acetic acid in the mobile phase increased from 0.2 to 1% (FIG. 3B, FIG. 3C and FIG. 3D). FIG. 3 shows the LC-MS/MS spectra of 100 ng/mL human insulin showing the effects of mobile phase gradient (varied 0.5 to 0.45 min) and percentage of acetic acid (HAC) as mobile phase moodier on peak sharpness and strength. Phase A=water; phase B=acetonitrile. FIG. 3A: Gradient, 15% to 40%; HAC, 0.2%; peak intensity=1.6×10³; FIG. 3B: Gradient, 15% to 70%; HAC, 0.2%; peak intensity=2.2×10⁴; FIG. 3C: Gradient, 15% to 70%; HAC, 0.5%; peak intensity=3.0×10⁴; FIG. 3D: Gradient, 15% to 70%; HAC, 1%; peak intensity=3.0×10⁴.

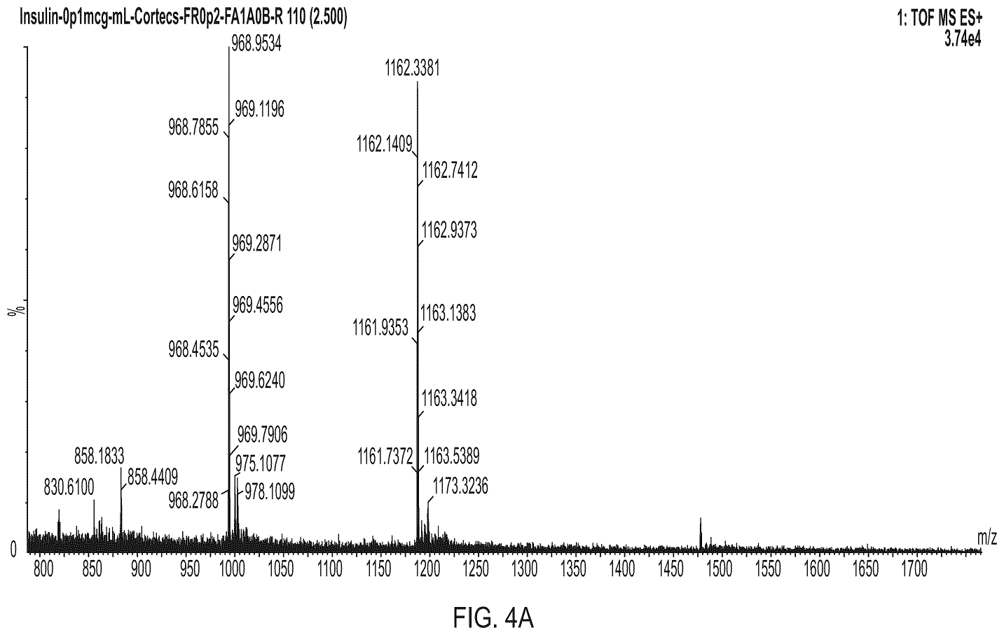

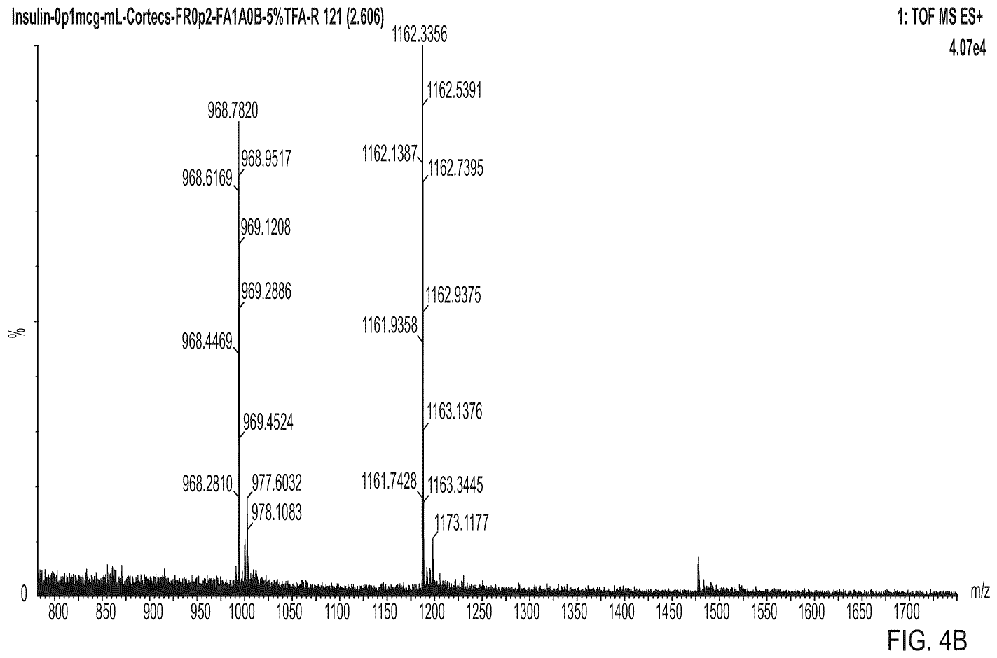

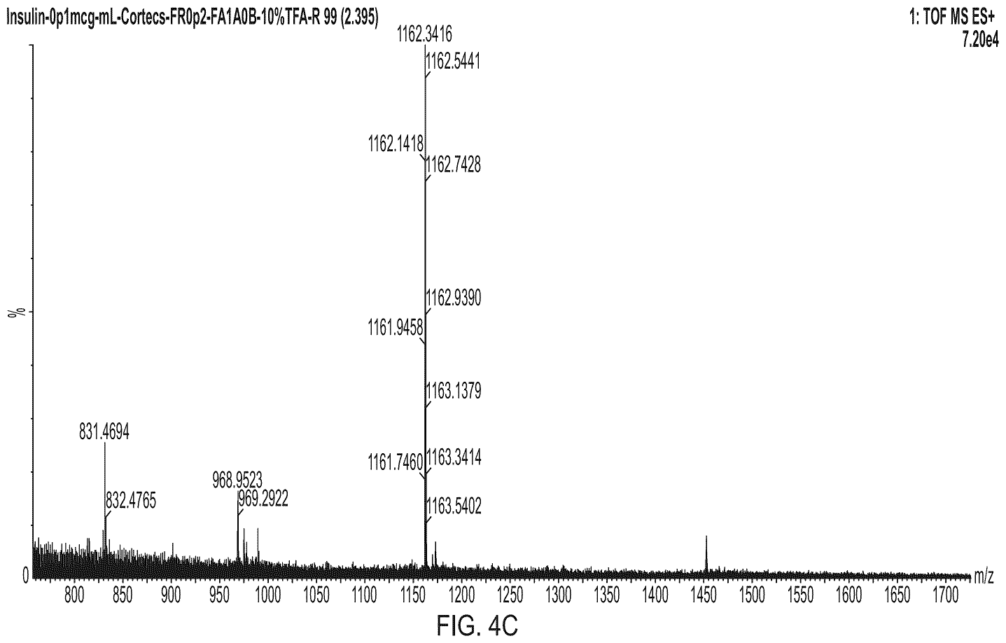

The effect of THF as a mobile phase modifier was also investigated. However, it did not increase the intensity of the insulin LC-MS/MS peak sharpness or strength. Interestingly, investigation of the fragmentation pattern using a Q-TOF, showed that it suppressed the M6+ ion especially at high concentration (FIG. 4). FIG. 4 shows the effect of percentage of THF as a mobile phase modifier on insulin ionization pattern. FIG. 4A: 0% THF; FIG. 4B: 5% THF; FIG. 4C: 10% THF.

### I. Assessment of Method Specificity Matrix Effect and Sensitivity.

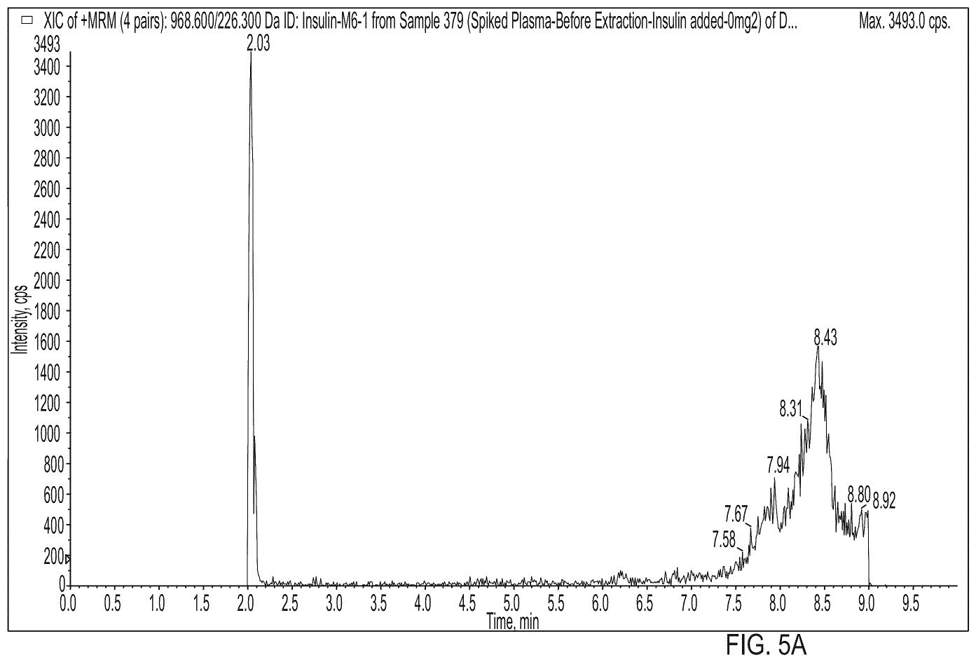

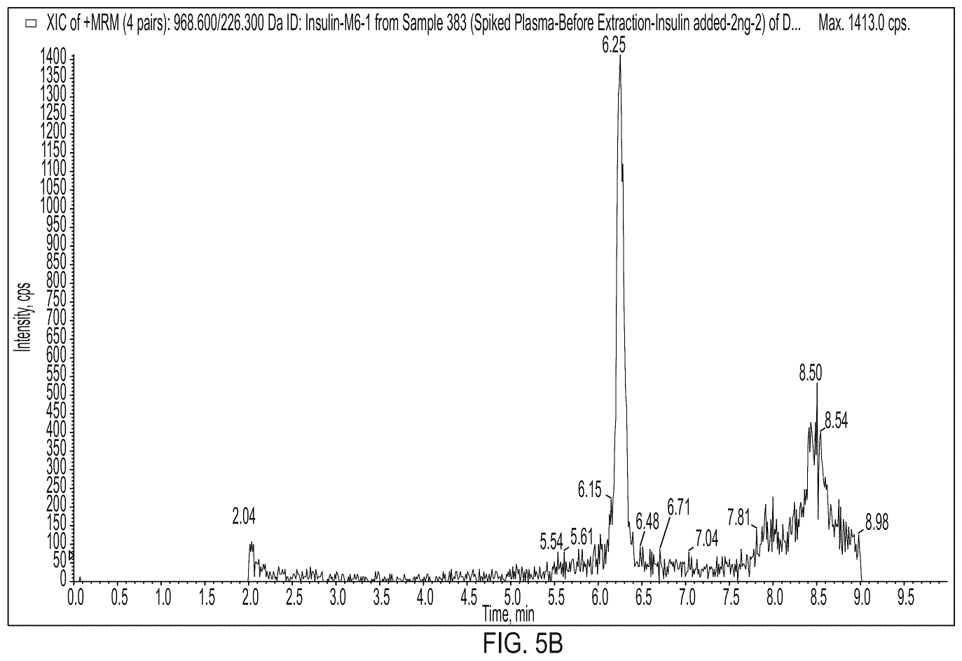

Although insulin sample extraction and purification involves protein precipitation steps, due the presence of similar peptides and other components, assessment of insulin the tissue extracts might have problems of specificity, selectivity and matrix effect. The selectivity and specificity of the developed method in biological sample was assessed using plasm as a surrogate matrix and spiking it with insulin. At the retention times of both human and bovine insulins, no −MS/MS peak was detected indicating that the method is selective to insulin; FIG. 5 provides LC-MS/MS spectra of rat plasma (FIG. 5A) and rat plasma (FIG. 5B) spiked with human insulin.

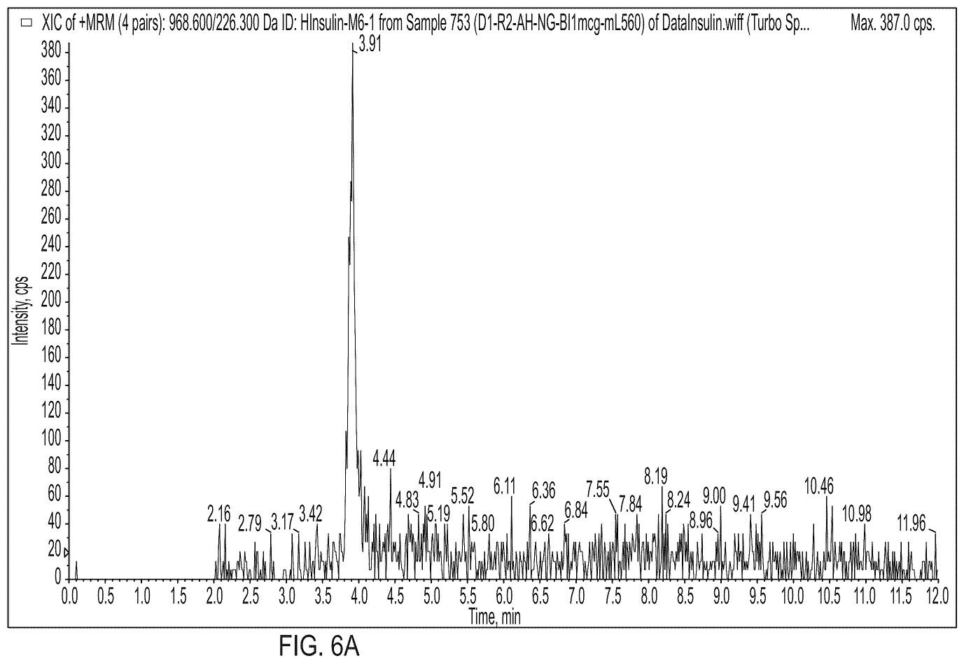

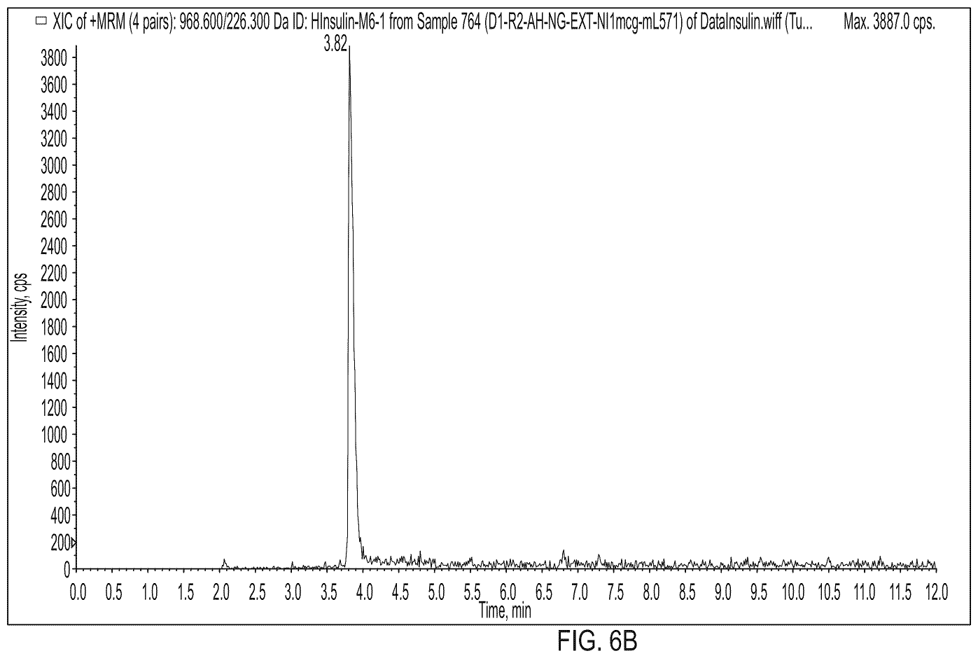

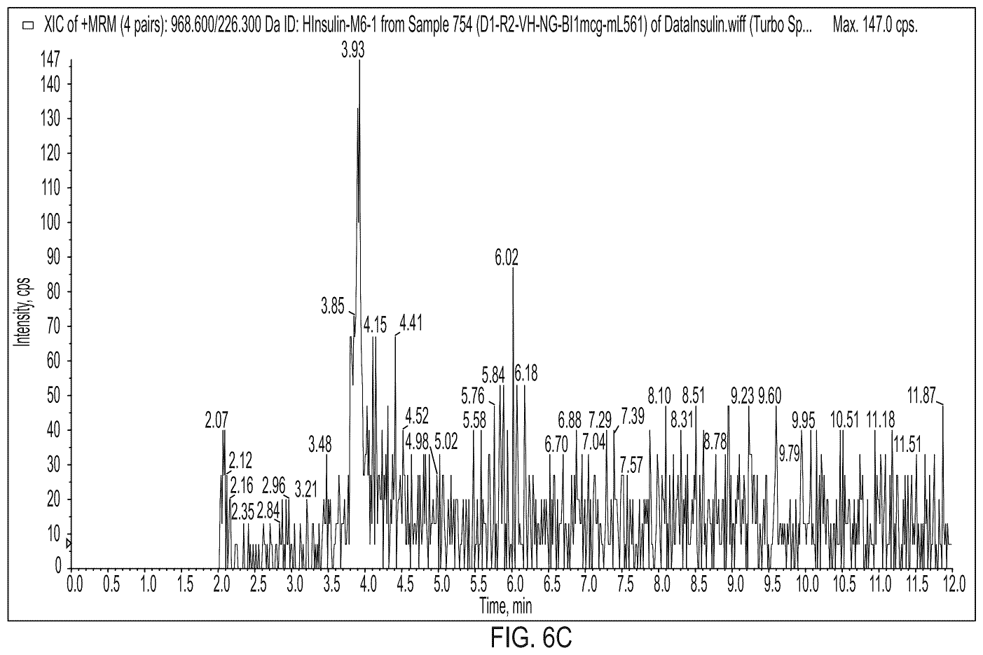

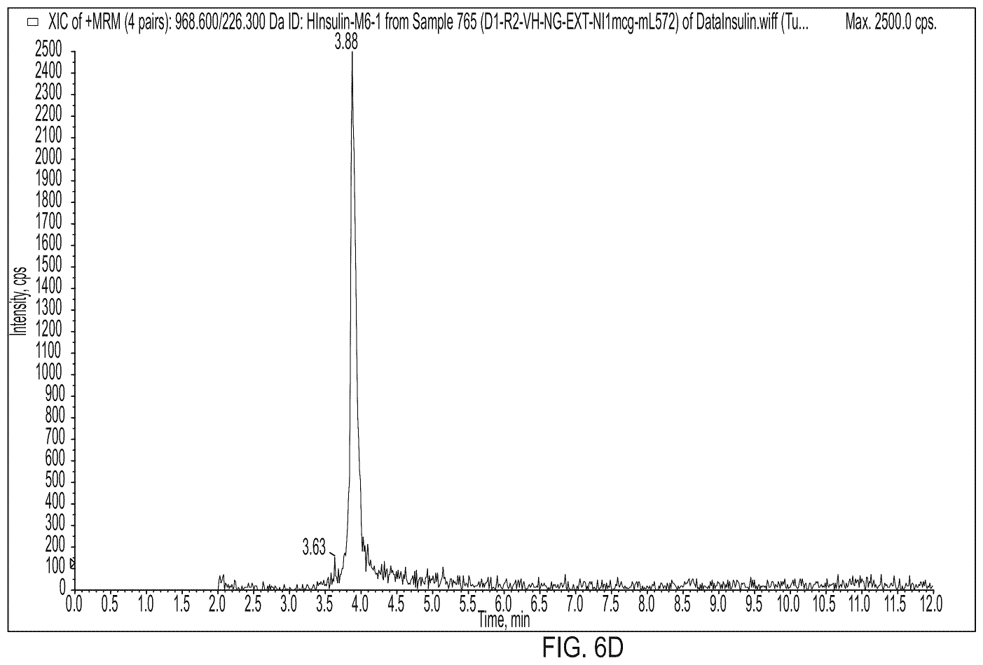

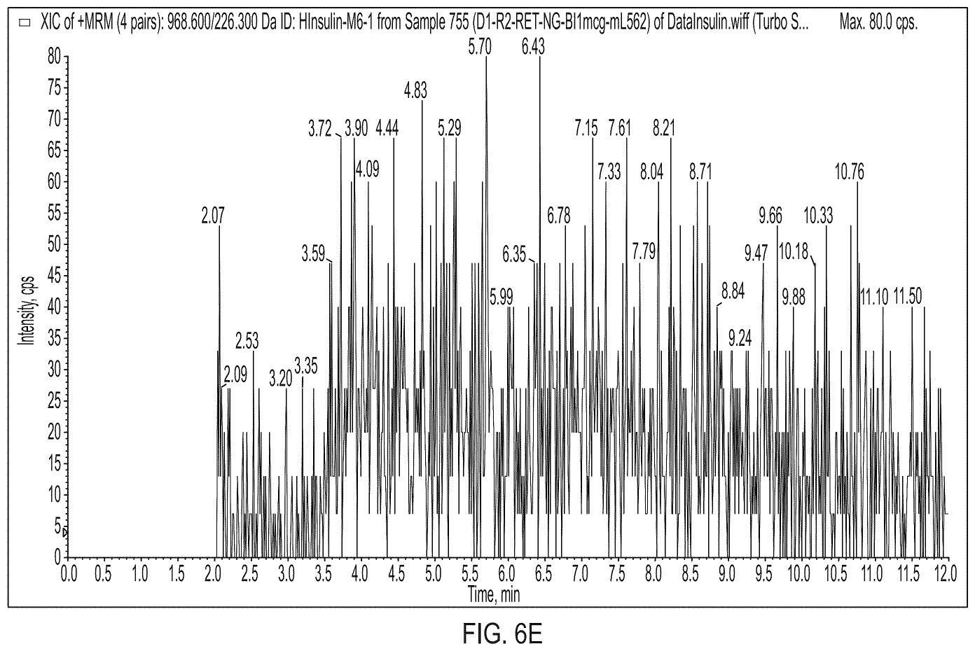

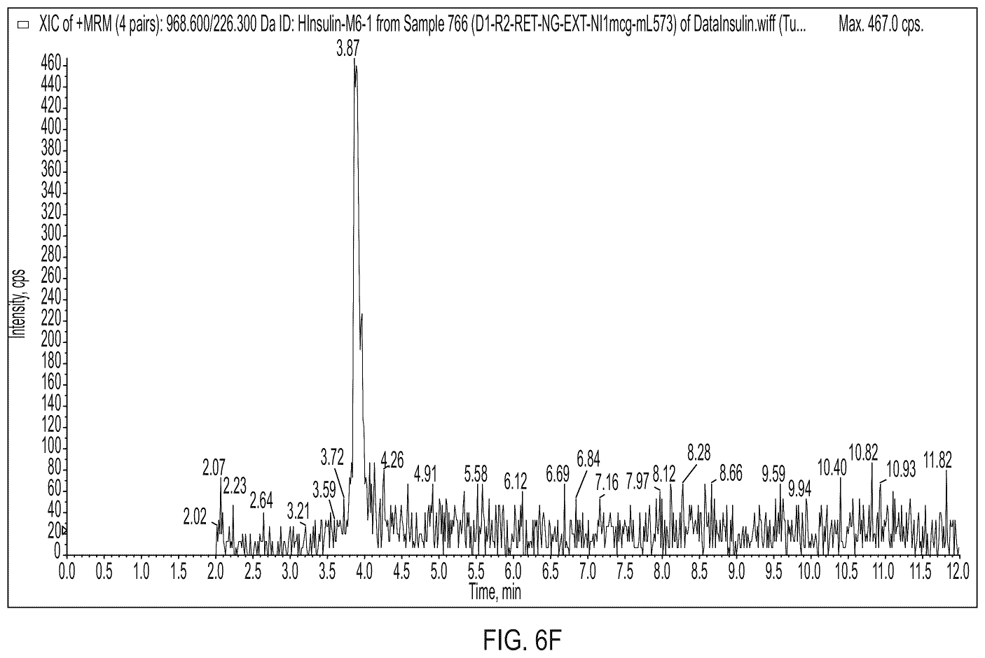

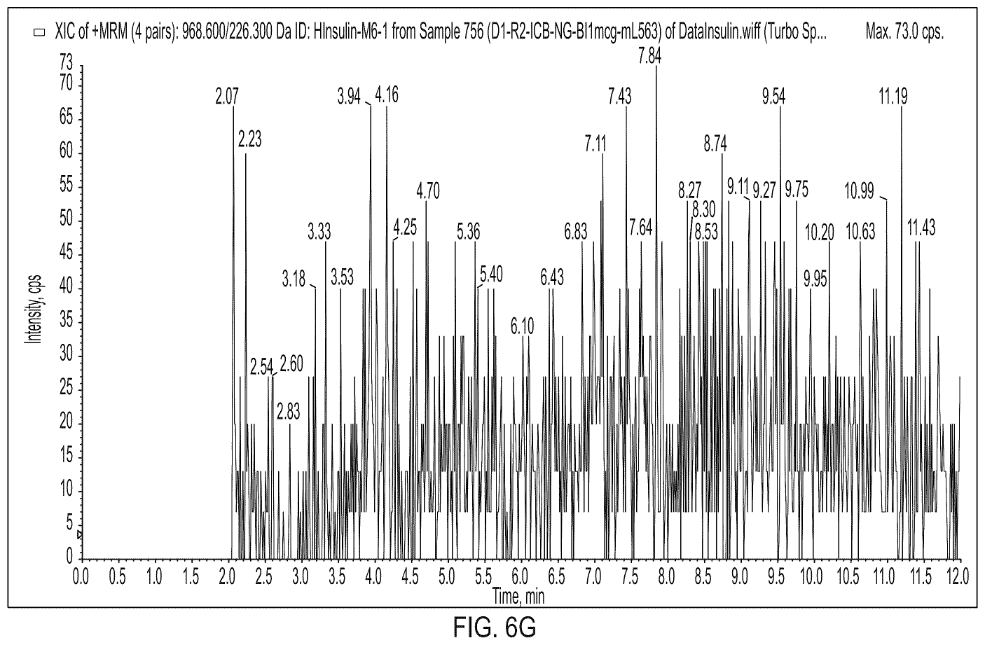

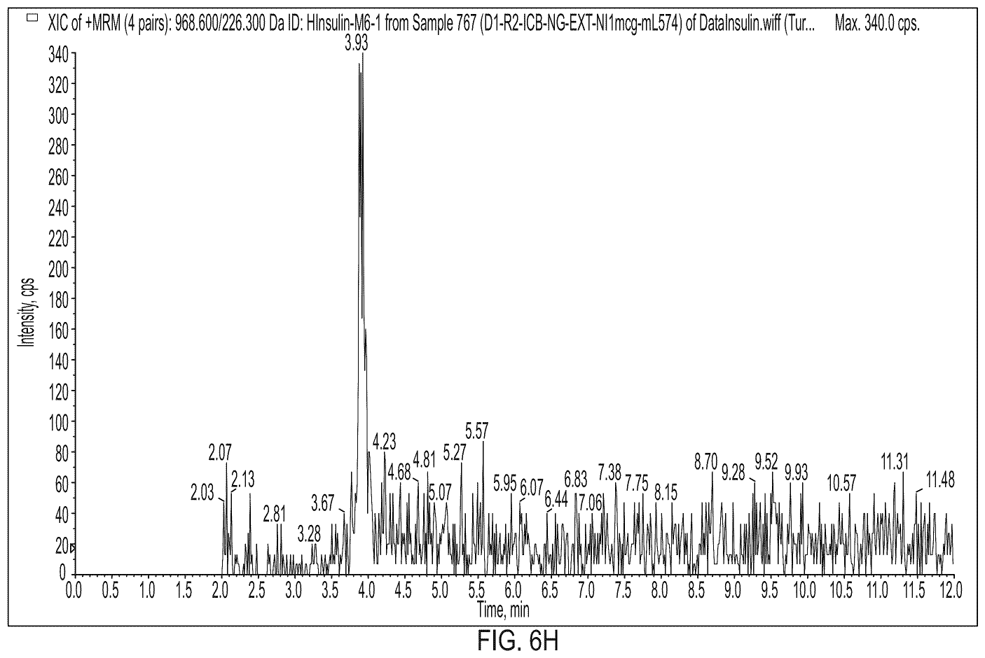

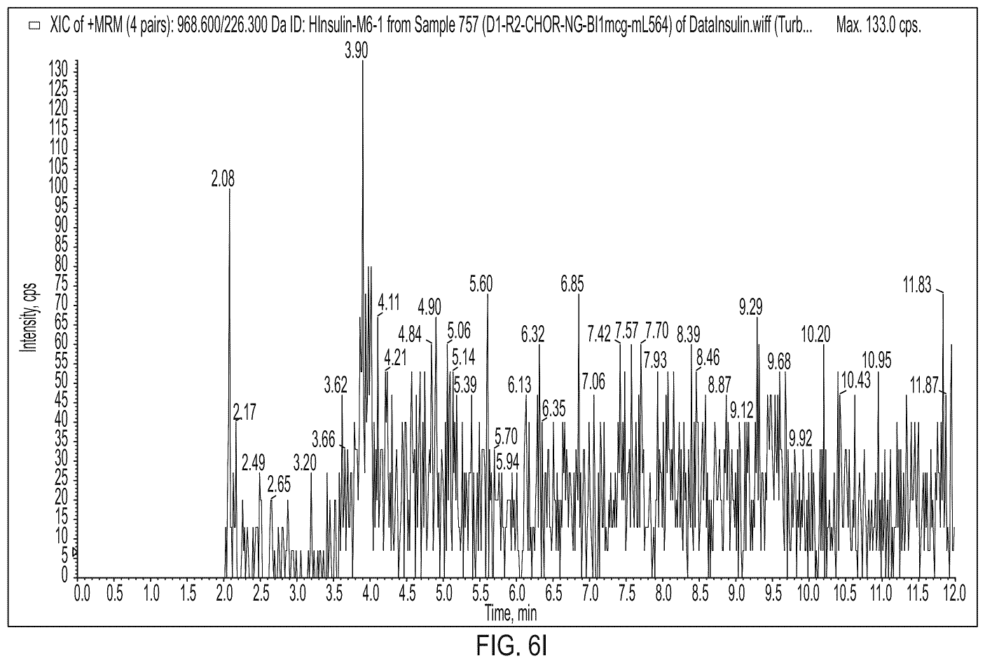

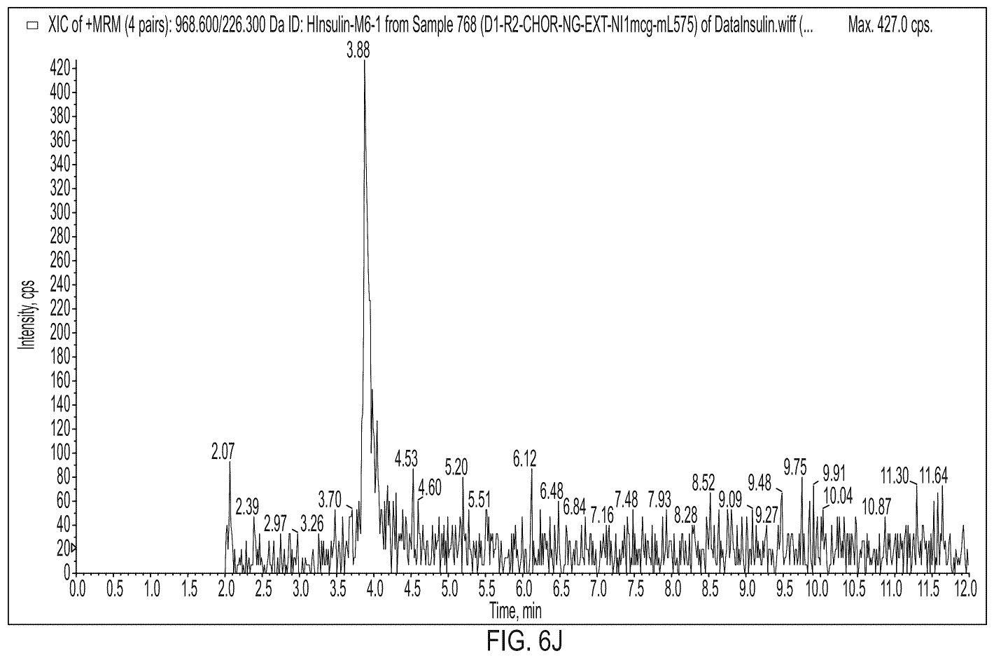

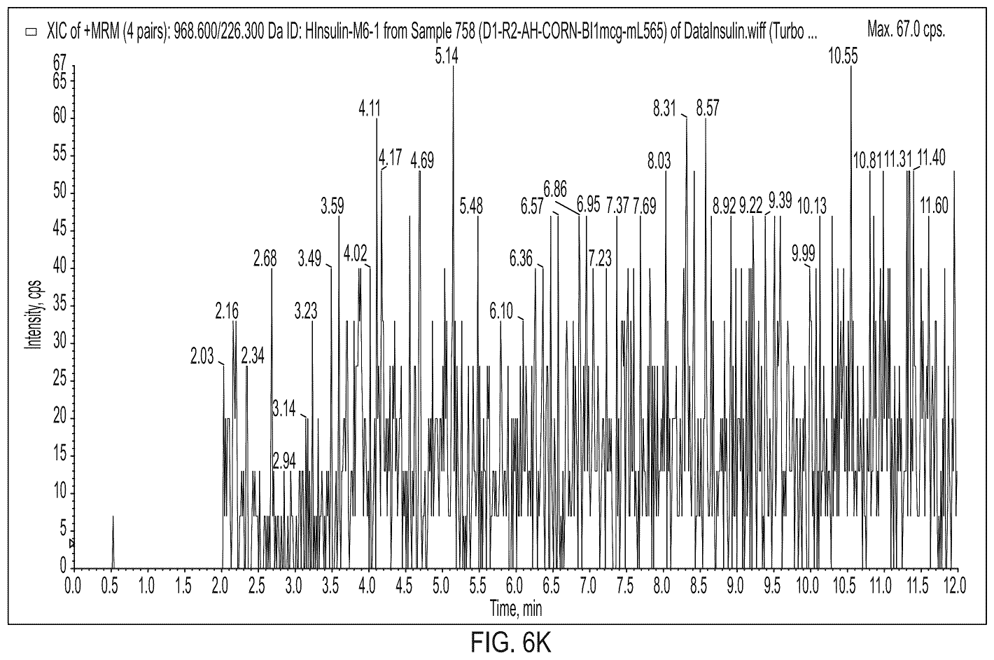

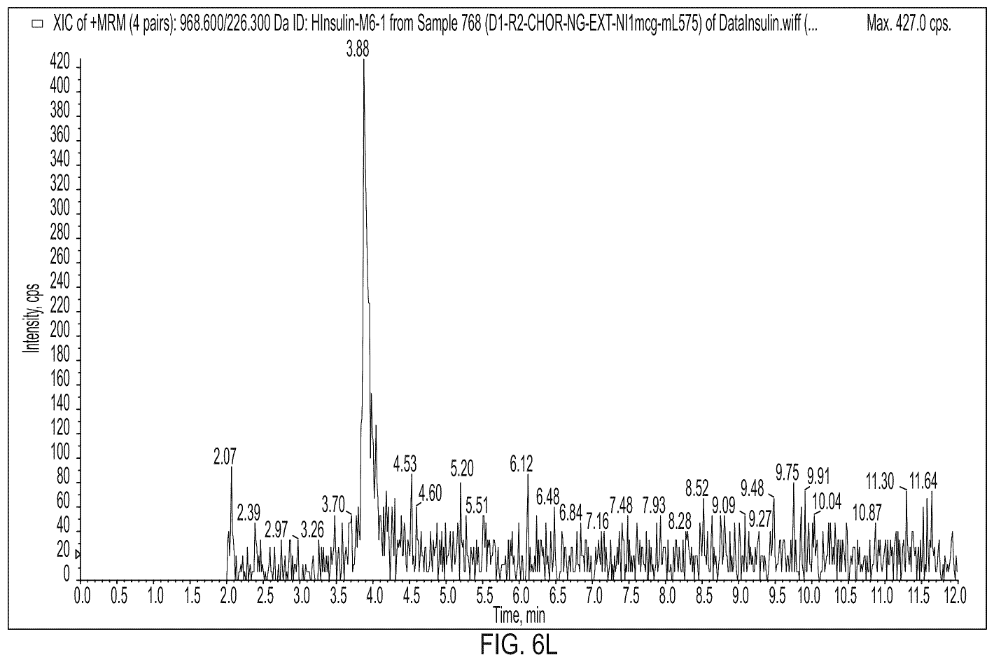

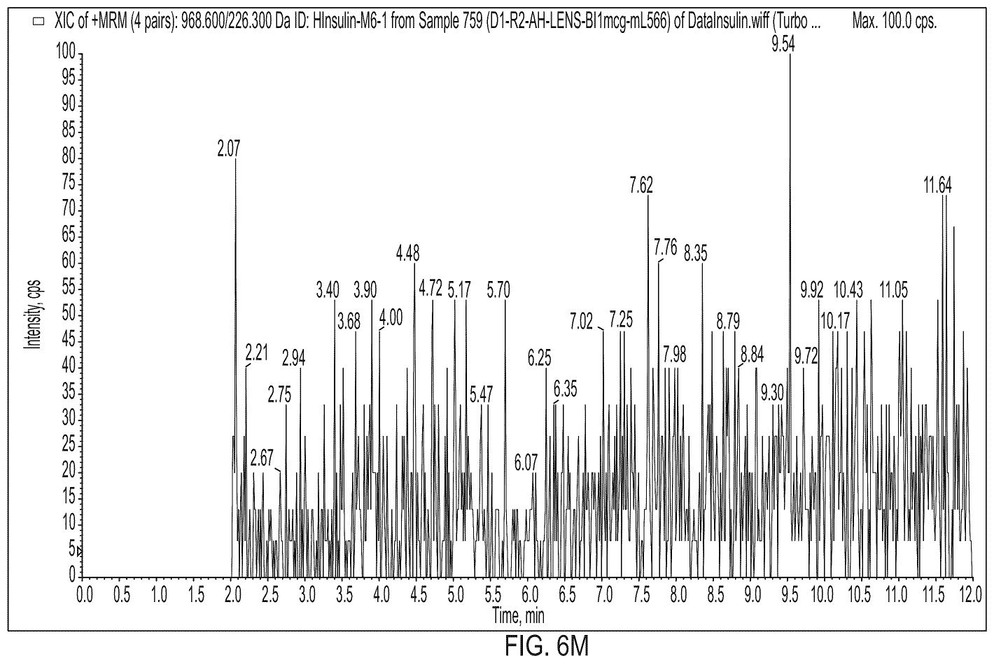

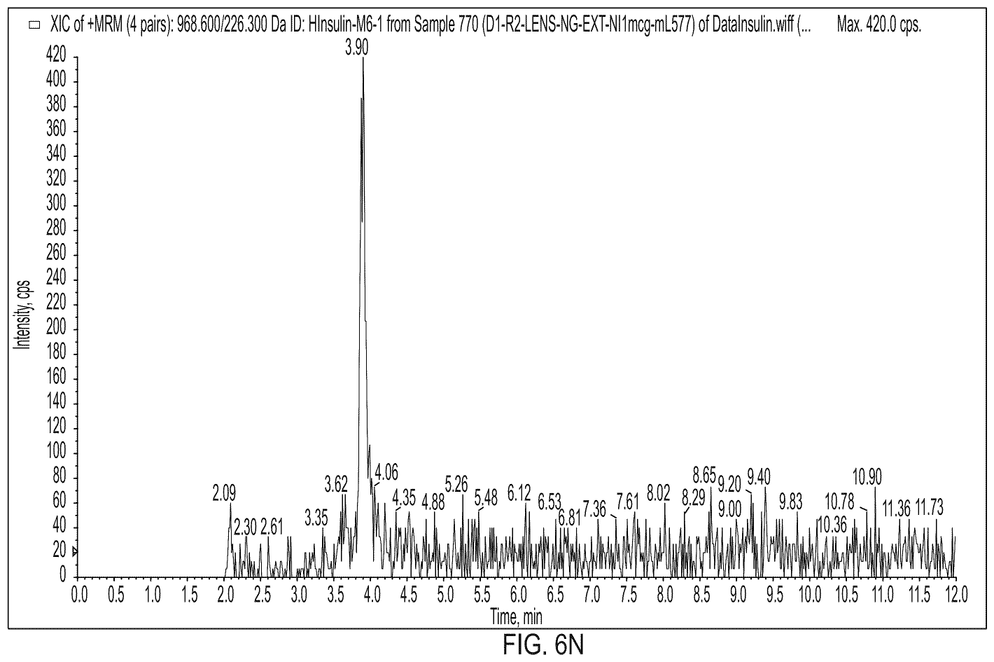

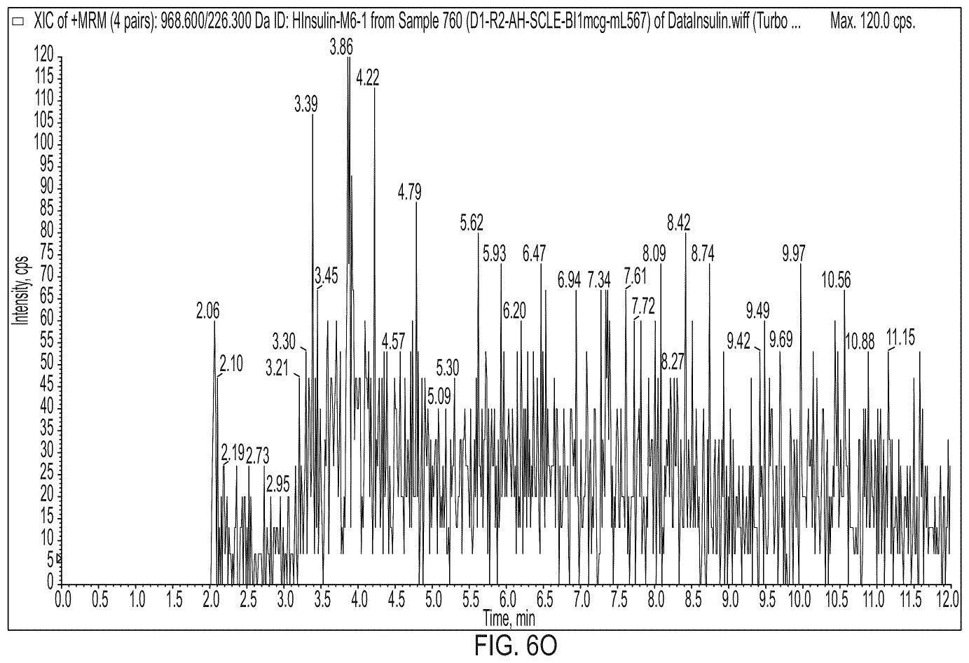

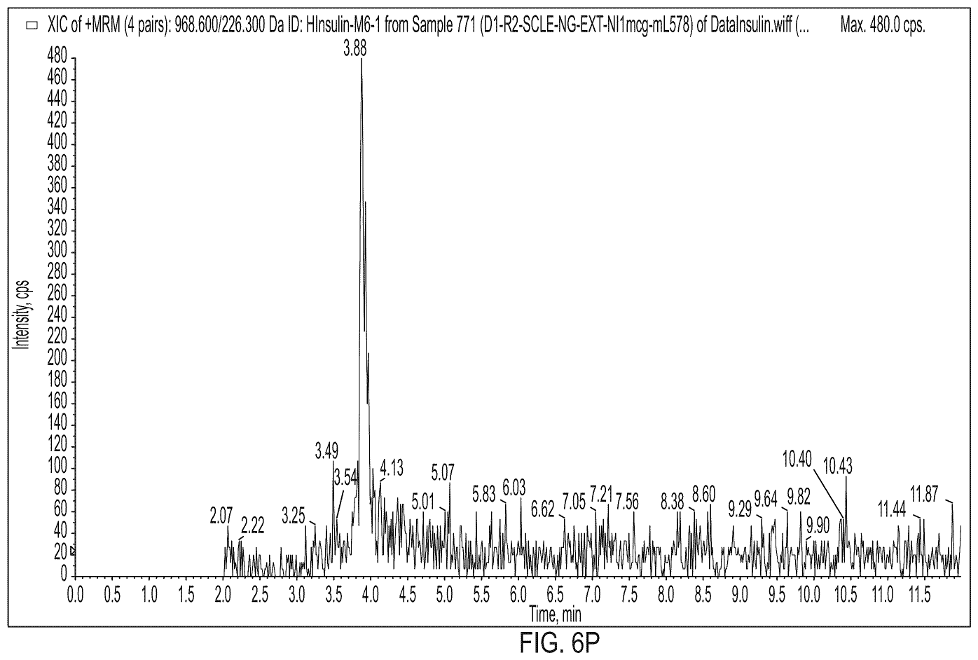

Similarly, the selectivity and specificity of the method in the different eye tissue was investigated and no LC-MS/MS peak was detected within the retention time of both human and bovine albumins. However, the intensities of the spiked samples was significantly low compared to the non-spiked samples showing a significant matrix effect and sample loss during sample preparation. To minimize the matrix effect, the tissue extract was further subjected to solid phase extraction (SPE) and in all the ocular tissues isolated SPE significantly reduced matrix effect and improved method sensitivity (FIG. 6). FIG. 5 provides LC-MS/MS spectra of insulin extracted from different eye tissues obtained with and without solid phase extraction. Accordingly, a calibration curve describing the relationship between concentration and AUC was constructed using plasma as a surrogate model. The linearity of the method was 50 to 2500 ng/mL (R²=0.9991) with accuracy more than 85%. FIG. 6 provides LC-MS/MS spectra of insulin extracted from different eye tissues obtained with and without solid phase extraction. FIG. 6A: AH, before SPE; FIG. 6B: SPE, after SPE; FIG. 6C: VH, before SPE; FIG. 6D: VH, after SPE; FIG. 6E: retina, before SPE; FIG. 6F: retina, after SPE; FIG. 6G: I/CB, before SPE; FIG. 6H: I/CB, after SPE; FIG. 6I: choroid, before SPE; FIG. 6J: choroid, after SPE; FIG. 6K: cornea, before SPE; FIG. 6L: cornea, after SPE; FIG. 6M: lens, before SPE; 6N: lens, after SPE; FIG. 6O: sclera, before SPE; FIG. 6P: sclera, after SPE.

### Example 2. Assessment of Non-Specific Insulin Binding to Vials and Vial Inserts

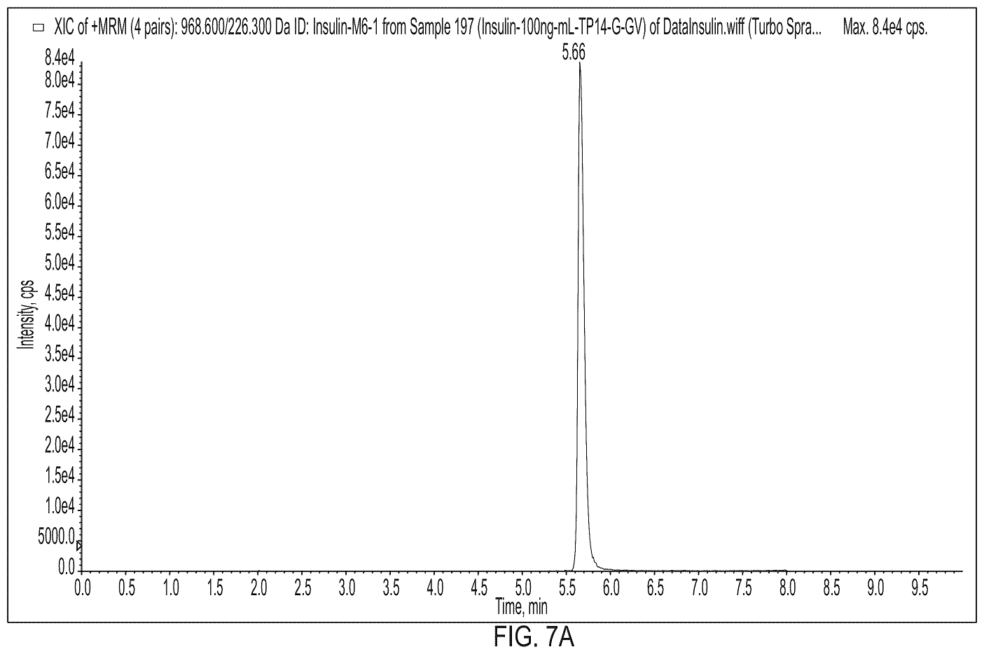

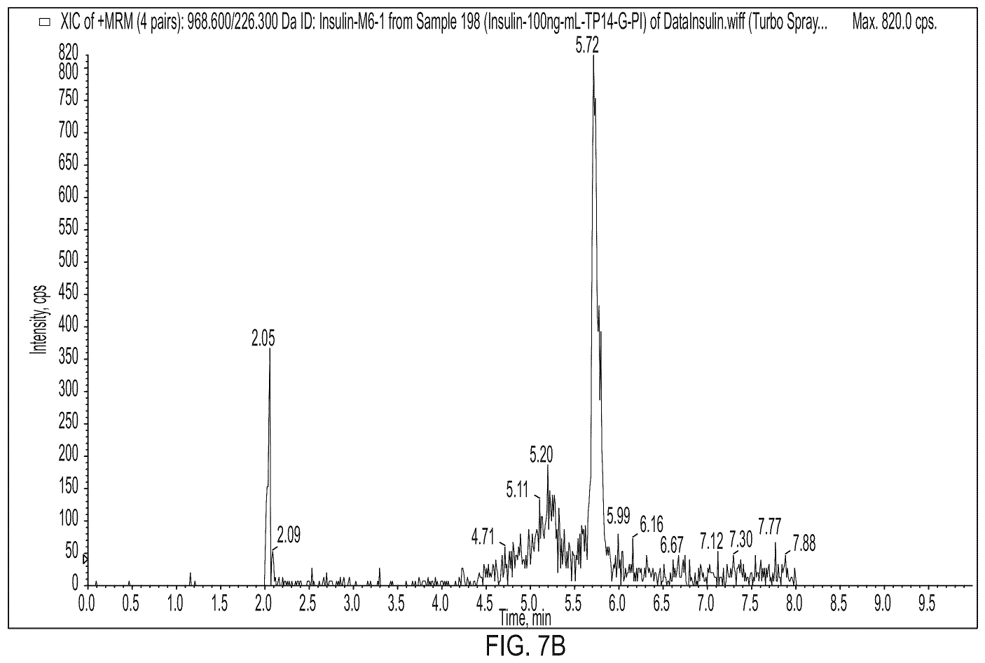

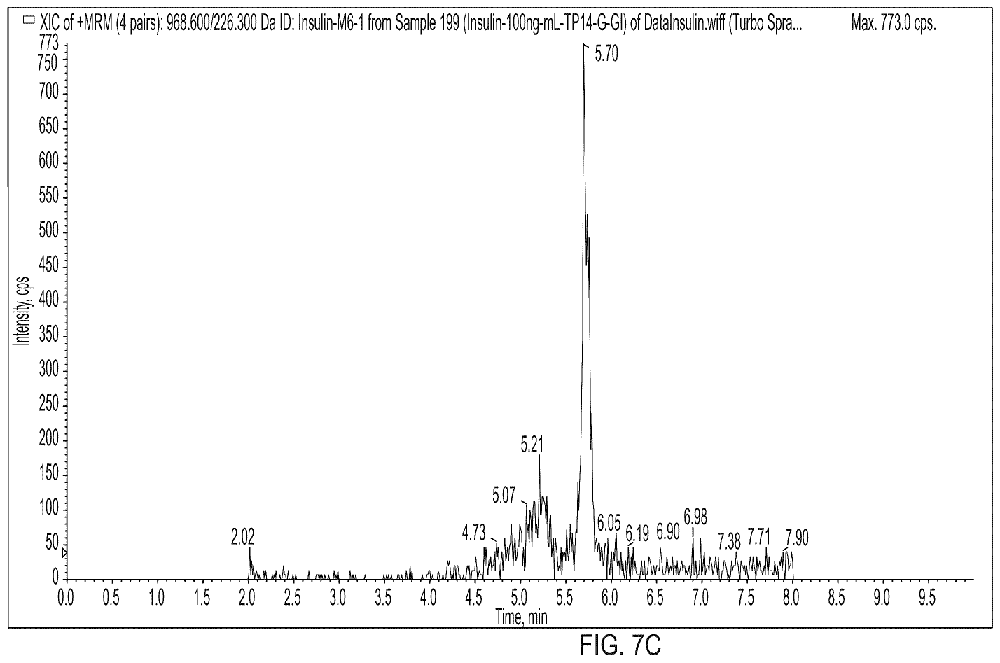

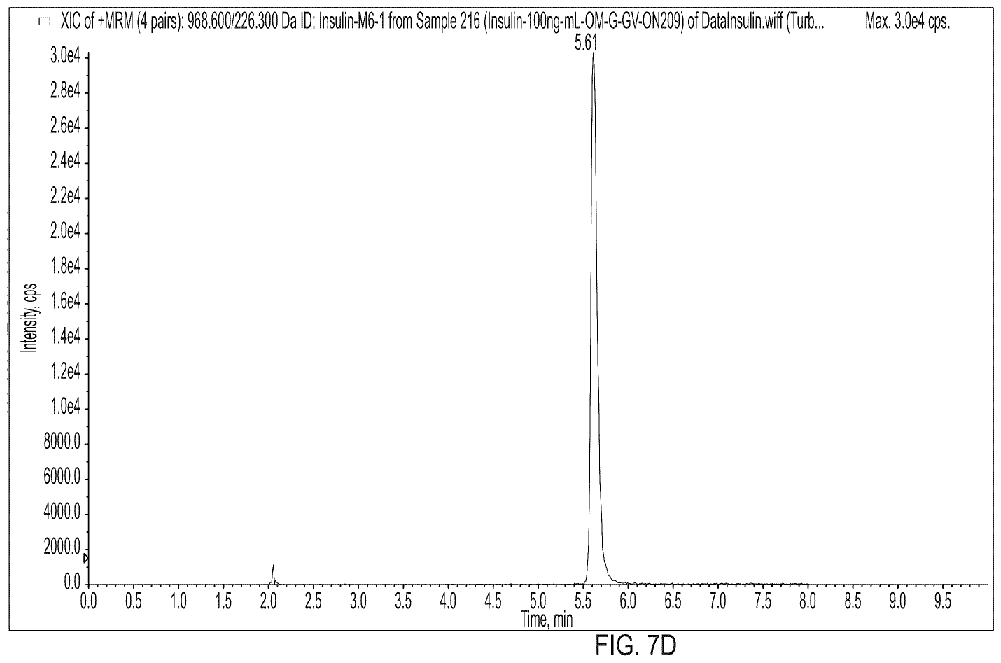

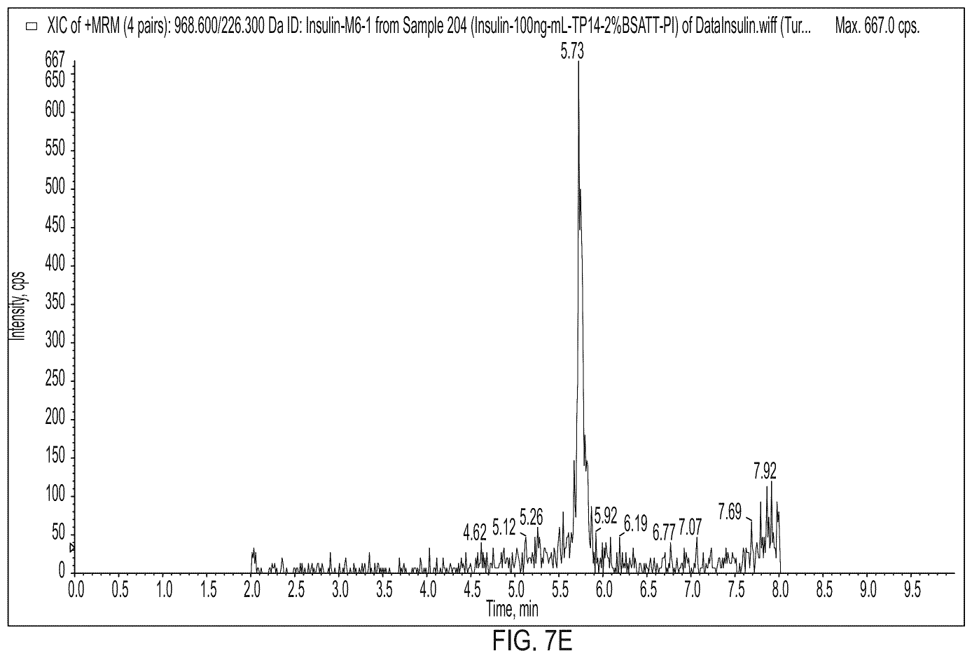

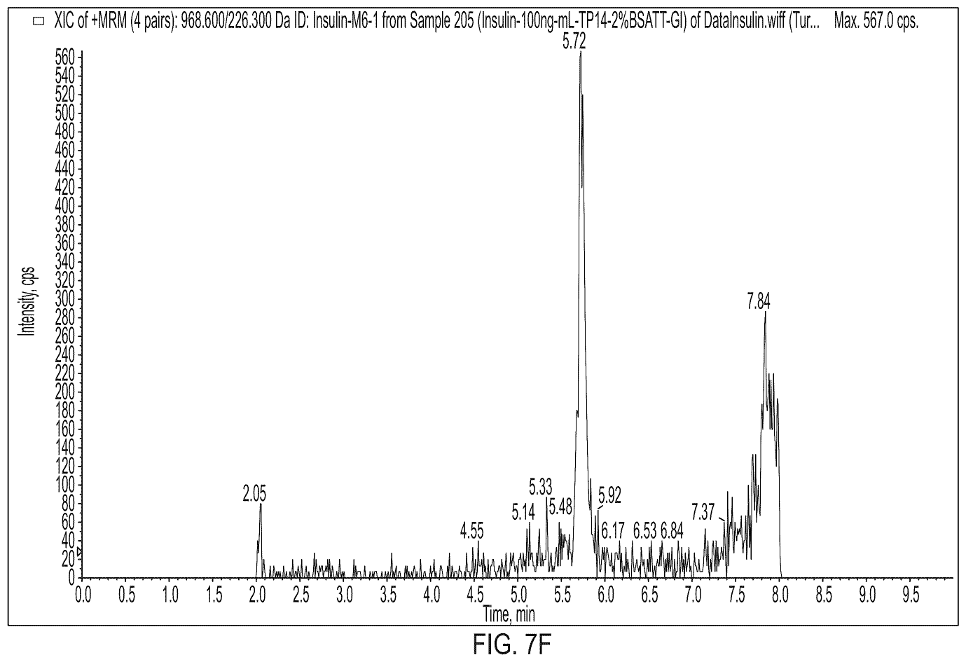

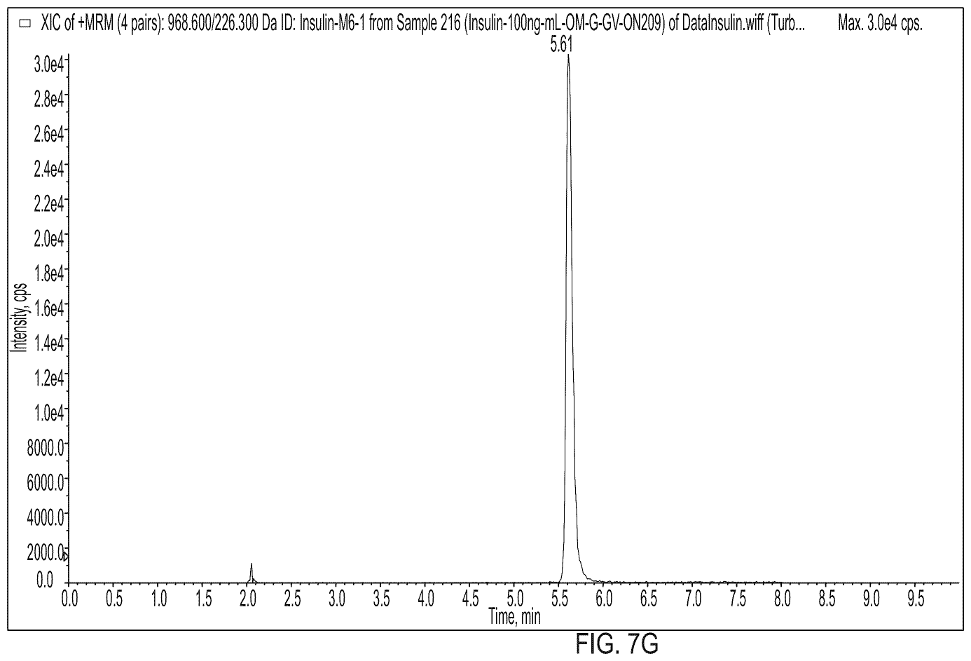

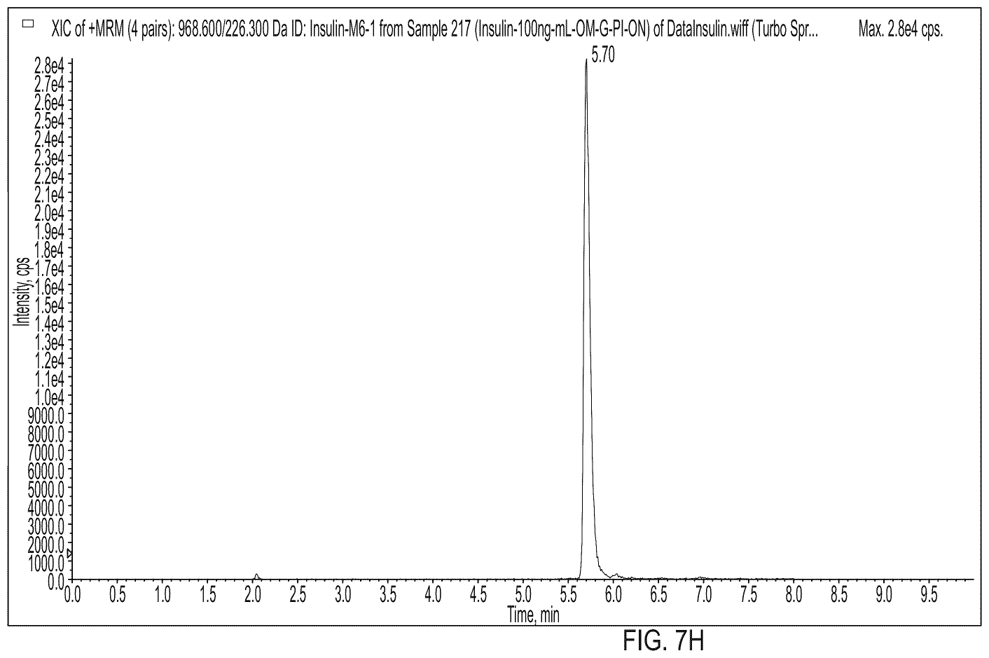

When working with biological samples low volumes of solvent are used along with glass inserts. However, at low concentration, insulin underwent a significant nonspecific binding to the surfaces of glass and plastic inserts and resulted in about 100-fold reduction in insulin peak intensity when compared to glass vials (See FIG. 7A, FIG. 7B, and FIG. 7C). Using a low percentage of BSA in the protein solution or coating the vials or inserts using BSA is reported to minimize non-specific protein adsorption. However, using 0.1% BSA (FIG. 7D) or coating both plastic and glass vials with 2% BSA overnight prior to their use (FIG. 7E and FIG. 7F) did not prevent non-specific insulin binding to glass and plastic inserts.

In addition, the extent of non-specific insulin adsorption to the different surfaces with time was investigated by analyzing the same samples kept overnight in their respective containers. In both glass vial and glass insert, the extent of insulin adsorption increased with time and it was irreversible (FIG. 7G, FIG. 7I). Interestingly, insulin adsorption to the plastic inserts was transient and the peak intensity increased considerably (FIG. 7H) significantly overnight, indicating that the adsorption process is partially reversible.

Therefore, during insulin measurement in different vials and inserts, the time dependent insulin adsorption should be taken into consideration. In addition, the effect of percentages of acetic acid and isopropanol in water on the extent of insulin adsorption in plastic and glass inserts was investigated and higher percentage of acetic acid and isopropanol minimized insulin adsorption and increased peak intensity about 8-fold (see Table 1, below).

TABLE 1
50 ng/mL insulin LC-MS/MS peak intensity changes as
a function of percent acetic acid and isopropanol.

| | Plastic | Plastic insert | Glass | Glass insert |
|---|---|---|---|---|
| Solvent/ insert | insert | overnight | insert | overnight |
| 0% HAC | 318 | 296 | 276 | 150 |
| 0.1% HAC, 0% IPN | 308 | 390 | 354 | 203 |
| 1% HAC, 0% IPN | 1205 | 951 | 1166 | 1049 |
| 2% HAC, 0% IPN | 1449.5 | 1027 | 1352 | 1286 |
| 3% HAC, 0% IPN | 1406 | 1095 | 1545 | 1482 |
| 2% HAC, 10% IPN | 1407 | 1187 | — | — |
| 2% HAC, 20% IPN | 2328 | 1947 | — | — |
| 2% HAC, 30% IPN | 2773 | 2329 | — | — |

Therefore, solvent with 2% of acetic acid was used for sample processing as described in the methodology part. FIG. 7A: MS spectrum of 100 ng/mL insulin in a glass vial. Peak intensity=8.0×10⁴; FIG. 7B: MS spectrum of 100 ng/mL insulin in a plastic insert. Peak intensity=820; FIG. 7C: MS spectrum of 100 ng/mL insulin in a glass insert. Peak intensity=773; FIG. 7D MS spectrum, of 100 ng/mL insulin in 0.1% BSA in a plastic insert. Peak intensity=700; FIG. 7E: MS spectrum of 100 ng/mL insulin in a BSA coated plastic insert. Peak intensity=640; FIG. 7F: MS spectrum of 100 ng/mL insulin in a BSA coated glass insert. Peak intensity=640; FIG. 7G: MS spectrum of 100 ng/mL insulin kept in a glass vial overnight. Peak intensity=3.0×10⁴; FIG. 7H: MS spectrum of 100 ng/mL insulin kept in a plastic insert overnight. Peak intensity=2.8×10⁴; FIG. 7I: MS spectrum of 100 ng/mL insulin kept in a glass insert overnight. Peak intensity=140.

### Example 3. Macromer Synthesis

Dextran (poly lactic acid) hydroxy-ethyl-methacrylate (Dex-PLA-HEMA) and dextran (poly ε-caprolactone) hydroxy-ethyl-methacrylate (Dex-PCL-HEMA) precursors (PLA-HEMA and PCL-HEMA) were synthesized according to known methods. See Table 2, below.

TABLE 2
Characteristics of PLA-HEMA and PCL-HEMA precursors.

| Example | Precursor (theoretical DP) | Actual DP | Yield (%) |
|---|---|---|---|
| 1 | PLA-HEMA_DP 2 | 4 | 29 |
| 2 | PLA-HEMA_DP 4-B#1 | 6 | 52.5 |
| 3 | PLA-HEMA_DP 4-B#2 | 6 | 64.2 |
| 4 | PLA-HEMA_DP 4-B#3 | 6 | 61.5 |
| 5 | PLA-HEMA_DP 6 | 8 | 85.2 |
| 6 | PCL-HEMA_DP 4 | 6 | 73.5 |

From the precursors, oligolactate-(2-hydroxyetheyl methacrylate) (PLA-HEMA) grafted dextran macromers (Dex-PLA-HEMA) were synthesized with varying degrees of polymerization (DP) and degrees of substitution (DS) according to known methods per Huang et. al (2015). Both of the Dex-PLA-HEMA and the Dex-PCL-HEMA macromers were obtained following the same procedure. The DP and DS of the macromers were estimated from 1H NMR (Bruker, 500 MHz) spectra obtained in deuterated DMSO. Dex-PLA-HEMA macromers with different DP (the number of lactic acid in the PLA segment) and DS (the number of PLA groups per 100 glucose unit of the dextran chain) were synthesized. See Table 3 and Table 4, below.

Whenever further purification of the macromer was necessary, the macromer was dissolved in 33.3% v/v ethanol (0.36 mg/mL) at 45° C. for 15 minutes, dialyzed for 1 hour in a dialysis membrane (Spectra/Por® Biotech Cellulose Ester Dialysis Membrane MWCO: 100-500 D, Spectrum Laboratories Inc.), filtered through a 0.2 μm glass filter and lyophilized.

TABLE 3
Theoretical and Practical DP/DS Values and Yield
of Dex-PLA-HEMA and Dex-PCL-HEMA Macromers.

| | | | | Solubility |
|---|---|---|---|---|
| Example | Macromer_theoretical DP/DS | Practical DP/DS | Yield (%) | (mg/mL) |
| 1 | Dex-HEMA_DS/10_B#3 | 6.9 | 116.6 | >20 |
| 2 | Dex-HEMA_DS/17_B#3 | 11.0 | 95.5 | >20 |
| 3 | Dex-HEMA_DS/33_B#3 | 17.9 | 91.2 | >20 |
| 4 | Dex-HEMA_DS/50_B#3 | 20.8 | 65.0 | >20 |
| 5 | Dex-PLA-HEMA_2/10 | 4/4.1 | 78.1 | >20 |
| 6 | Dex-PLA-HEMA_2/17 | 4/11.8 | 68.7 | >20 |
| 7 | Dex-PLA-HEMA_2/33_B#2 | 4/14.4 | 65.9 | >20 |
| 8 | Dex-PLA-HEMA_2/50_B#2 | 4/17.1 | 56.6 | >20 |
| 9 | Dex-PLA-HEMA_4/10* | 6/4.2 | 81.2 | >20 |
| 10 | Dex-PLA-HEMA_4/17* (B3) | 6/6.2 | 57.4 | >20 |
| 11 | Dex-PLA-HEMA_4/17_B2** | 6/8.7 | 93.2 | >20 |
| 12 | Dex-PLA-HEMA_4/33* | 6/6.2 | 41.8 | Soluble |
| 13 | Dex-PLA-HEMA_4/50* | 6/7.8 | 31.7 | Soluble |
| 14 | Dex-PLA-HEMA_6/10 | 8/4.6 | 81.7 | Soluble |
| 15 | Dex-PLA-HEMA_6/17 | 8/10.2 | 71.0 | Soluble |
| 16 | Dex-PLA-HEMA_6/33 | 8/22.6 | 61.0 | Insoluble |
| 17 | Dex-PLA-HEMA_6/50 | 8/28.1 | 46.0 | Insoluble |
| 18 | Dex-PLA-HEMA_10/10 | 12/2.3 | 81.5 | Insoluble |
| 19 | Dex-PLA-HEMA_10/17 | Crosslinked | | |
| 20 | Dex-PLA-HEMA_10/33p | 12/4.3 | 36.8 | Insoluble |
| 21 | Dex-PLA-HEMA_10/50 | 12/8.8 | 46.8 | Insoluble |
| 22 | Dex-PCL-HEMA_2/10 | 6/2.3 | 89.9 | >20 |
| 23 | Dex-PCL-HEMA_2/17 | 6/2.2 | 74.3 | >20 |
| 24 | Dex-PCL-HEMA_2/33 | 6/2.4 | 53.0 | >20 |
| 25 | Dex-PCL-HEMA_2/50 | 6/2.7 | 44.7 | >20 |
| 26 | Dex-PCL-HEMA_4/10 | 6/3.5 | 74.7 | Soluble |
| 27 | Dex-PCL-HEMA_4/17 | 6/4.1 | 68.3 | Soluble |
| 28 | Dex-PCL-HEMA_4/33 | 6/6.5 | 47.0 | Insoluble |
| 28 | Dex-PCL-HEMA_4/50 | 6/7.9 | 36.7 | Insoluble |

*reaction took place for 4 days,
**reaction took place for 10 days, partially crosslinked DP 0-crosslinked; crosslinker added.

TABLE 4
DP/DS and Solubility

| | | Practical | Solubility |
|---|---|---|---|
| Example | Theoretical DP/DS | DP/DS | in water |
| 1 | Dex-PLA-HEMA_2/10 | 4/4.1 | Soluble |
| 2 | Dex-PLA-HEMA_2/17 | 4/11.8 | Soluble |
| 3 | Dex-PLA-HEMA_2/33 | 4/14.4 | Soluble |
| 4 | Dex-PLA-HEMA_2/50 | 4/17.1 | Soluble |
| 5 | Dex-PLA-HEMA_6/10 | 8/4.6 | Soluble |
| 6 | Dex-PLA-HEMA_6/17 | 8/10.2 | Soluble |
| 7 | Dex-PLA-HEMA_6/33 | 8/22.6 | Insoluble |
| 8 | Dex-PLA-HEMA_6/50 | 8/28.1 | Insoluble |
| 9 | Dex-PCL-HEMA_4/10 | 6/3.5 | Soluble |
| 10 | Dex-PCL-HEMA_4/17 | 6/4.1 | Soluble |
| 11 | Dex-PCL-HEMA_4/33 | 6/6.5 | Insoluble |
| 12 | Dex-PCL-HEMA_4/50 | 6/7.9 | Insoluble |

### Example 5. Synthesis of Blank and Loaded Nanogels

Nanogels were synthesized by UV emulsion polymerization using Irgacure® 2959 as an initiator. These nanogels contained N-Isopropylacrylamide (NIPAAm) and dextran grafted oligolactate-(2-hydroxyetheyl methacrylate) (Dex-PLA-HEMA, DP=6 and DS=8.7) or dextran grafted oligocaprolactone-(2-hydroxyetheyl methacrylate) (Dex-PCL-HEMA, DP=6 and DS=4.1) macromer at varying weight ratio 8:1, 7:2, 6:3, 5:4 and 4:5, with and without acrylic acid (AA) or 2-aminoethyl methacrylate at 0, 2, 5, 7 or 10 mol % with respect to NIPAAm. During synthesis, NIPAAm (monomer) and Dex-PLA-HEMA or Dex-PCL-HEMA (macromer) were dissolved in water (10 to 25 mg/mL), and degassed for 10 minutes using N₂ gas. The solution was exposed to UV light at 320-500 nm wavelength (1 W/cm²) (EXFO, Inc.) under stirring at 300 rpm and 45° C. for 15 minutes.

In the case of insulin loaded nanogels, 15 wt % insulin was added to the reaction mixture after the insulin was dissolved by pH change (double concentration of insulin (3 mg/mL) was prepared by dissolving it in pH 2.5 HCl solution for 3 minutes followed by pH adjustment to about 7 using 0.1 N NaOH). In the case of Avastin-loaded nanogels, siRNA-loaded nanogels (0.5 nmol) and anti-TNFα-loaded nanogels, 1 to 37.5 wt % Avastin, 6.65×10⁻³ wt % siRNA or 3×10⁻³ wt % anti-TNFα, respectively was added to the reaction mixture before the UV polymerization. See FIG. 1, which illustrates the nanogel general synthesis process. After the synthesis, the nanogels were dialyzed in DI water through a dialysis membrane (Spectra/Por® 6 Dialysis Membrane MWCO: 50 kD, Spectrum Laboratories Inc.) for 5 hours, with frequent change of the dialysis media (every 1 hour). The purified nanogel dispersion was lyophilized to obtain dry nanogels.

Nanogels were produced using varying weight ratios of monomer to macromer (8:1, 7:2, 6:3, 5:4, and 4:5) using 0, 2, 5, 7 and 10 mole % of comonomer acrylic acid (A) or 2-aminoethyl methacrylate (B) with respect to NIPAAm as nanogel surface and bulk modifier. The different nanogels were identified as 81A, 72A, 63A, 54A, and 45A, respectively, when acrylic acid was used, as 81B, 72B, 63B, 54B, and 45B when 2-aminoethyl methacrylate (B) was used as nanogel surface and bulk modifier. Nanogels with neither are referred to as 81, 72, 63, 54, and 45. The nanogel terminology also reflected the type (PLA for PLA based macromer and PCL for PCL based macromers) and the DP/DS of the macromer used.

The effect of the initiator concentration and reaction/UV exposure time on the characteristics of PLA-72A DP/DS-4/17 was investigated as follows, prior to optimizing the effect of other parameters. Nanogel yield and particle size increased considerably with increasing initiator concentration, particularly up to 1.0% and 1.5% of the initiator concentration for yield and particle size, respectively. Nanogels were synthesized for about 15 to 30 minutes.

Data on yield, size, and size distribution obtained by dynamic light scattering. See Table 5, below, for results for reaction yield, particle diameter (size) and polydispersity index (PDI).

TABLE 5
Effect of initiator concentration on PLA-72A_DP/DS-6/8.7 nanogel
yield, size and size distribution (measured at 37° C.).

| | Irgacure | | | |
|---|---|---|---|---|
| No | conc. (%) | Yield (%) | Size (nm) | PDI |
| 1 | 0.1 | 61.3 | 68.2 (3.6) | 0.235 (0.043) |
| 2 | 0.2 | 70.0 | 81.2 (0.4) | 0.154 (0.008) |
| 3 | 0.5 | 81.8 | 131.4 (0.2) | 0.106 (0.013) |
| 4 | 1 | 88.4 | 158 (1.0) | 0.089 (0.007) |
| 5 | 1.5 | 89.0 | 172.7 (1.0) | 0.080 (0.004) |
| 6 | 2 | 89.2 | 170.0 (1.4) | 0.079 (0.010) |

At 1% Irgacure, the yield increased and the particle size decreased slightly when the reaction/UV exposure time increased up to 15 minutes, measured at 37° C. See Table 6, below. Afterwards, the yield decreased and size increased slightly. The amount of unreacted NIPAAM, recovered from the dialysis media after 4 hours of nanogel dialysis and quantitated by UPLC (AUC=43098C−291.49, R²=0.9999), also showed early completion of the reaction.

TABLE 6
Effect of reaction time on yield, size, and size
distribution of PLA-72_DP/DS-6/8.7 nanogels.

| | Reaction | NIPAAM | | | |
|---|---|---|---|---|---|
| | time | consumed | Yield | | |
| No | (minutes) | (%) | (%) | Size (nm) | PDI |
| 1 | 5 | 90.1 | 89.7 | 171.6 (0.6) | 0.091 (0.013) |
| 2 | 10 | 92.7 | 91.2 | 175.6 (1.2) | 0.116 (0.014) |
| 3 | 15 | 91.0 | 95.2 | 157.9 (1.4) | 0.078 (0.015) |
| 4 | 20 | 92.6 | 93 | 166.2 (0.8) | 0.082 (0.019) |
| 5 | 25 | 91.2 | 92.2 | 150.8 (0.6) | 0.091 (0.005) |
| 6 | 30 | 92.9 | 92.7 | 149.0 (1.4) | 0.086 (0.013) |
| 7 | 45 | 93.0 | 90.2 | 161.3 (2.4) | 0.080 (0.012) |
| 8 | 60 | 92.3 | 91.6 | 170.6 (0.4) | 0.078 (0.011) |

TABLE 7
Characteristics of PLA based unloaded nanogels synthesized by UV
polymerization for in vitro nanogel cytotoxicity experiments.

| | | | 25° C. | | 37° C. | |
|---|---|---|---|---|---|---|
| | | Yield | Size (SD) | PDI | Size (SD) | PDI |
| No | Nanogel | (%) | (nm)* | (SD) | (nm)* | (SD) |
| 1 | PLA-81A_DP/DS-6/8.7 | 83.8 | 35.7 (5.7) | 0.546 (0.045) | 146.5 (0.6) | 0.093 (0.005) |
| 2 | PLA-72A_DP/DS-6/8.7 | 81.6 | 33.6 (8.7) | 0.551 (0.337) | 167.5 (1.2) | 0.098 (0.005) |
| 3 | PLA-63A_DP/DS-6/8.7 | 82.1 | 39.5 (3.1) | 0.355 (0.093) | 175.3 (1.4) | 0.088 (0.015) |
| 4 | PLA-54A_DP/DS-6/8.7 | 80.3 | 29.7 (3.4) | 0.376 (0.087) | 178.5 (4.9) | 0.112 (0.025) |
| 5 | PLA-45A_DP/DS-6/8.7 | 79.1 | 35.2 (1.1) | 0.317 (0.062) | 161.5 (1.4) | 0.186 (0.004) |

TABLE 8
Characteristics of 15% insulin-loaded PLA-based nanogels synthesized
by UV polymerization for in vitro insulin release study.

| | | | 25° C. | | 37° C. | |
|---|---|---|---|---|---|---|
| | | Yield | Size (SD) | PDI | Size (SD) | PDI |
| No | Nanogel | (%) | (nm) | (SD) | (nm) | (SD) |
| 1 | PLA-81A_DP/DS-6/8.7 | 75.9 | 32.9 (5.5) | 0.335 (0.110) | 113.7 (1.6) | 0.058 (0.032) |
| 2 | PLA-72A_DP/DS-6/8.7 | 70.4 | 35.0 (3.4) | 0.463 (0.056) | 212.1 (1.3) | 0.034 (0.015) |
| 3 | PLA-63A_DP/DS-6/8.7 | 75.5 | 44.6 (5.1) | 0.451 (0.047) | 140.4 (1.9) | 0.098 (0.022) |
| 4 | PLA-54A_DP/DS-6/8.7 | 57.6 | 25.0 (2.1) | 0.381 (0.199) | 253.5 (5.9) | 0.066 (0.020) |
| 5 | PLA-45A_DP/DS-6/8.7 | 53.9 | 27.1 (1.8) | 0.239 (0.088) | 246.5 (5.3) | 0.086 (0.013) |
| 6 | PLA-72A_DP/DS-6/4.2 | 75.4 | 193.1 (8.4) | 0.751 (0.023) | 135.2 (1.0) | 0.056 (0.010) |
| 7 | PLA-72A_DP/DS-6/6.2 | 74.8 | 235.5 (31.5) | 0.497 (0.068) | 119.1 (1.3) | 0.111 (0.011) |
| 8 | PLA-72A_DP/DS-6/7.8 | 74.5 | 204.7 (18.7) | 0.356 (0.034) | 126.8 (2.1) | 0.093 (0.009) |
| 9 | PLA-72A_DP/DS-4/4.1 | 81.4 | 137.6 (11.6) | 0.353 (0.066) | 83.1 (8.5) | 0.394 (0.079) |
| 10 | PLA-72A_DP/DS-4/13.1 | 77.4 | 191.6 (6.8) | 0.450 (0.014) | 177.6 (1.1) | 0.128 (0.011) |
| 11 | PLA-72A_DP/DS-8/4.6 | 78.9 | 37.7 (6.8) | 0.333 (0.176) | 152.4 (1.3) | 0.059 (0.008) |
| 12 | PLA-72A_DP/DS-8/10.2 | 78.0 | 225.0 (12.2) | 0.486 (0.02) | 149.5 (1.9) | 0.050 (0.022) |
| 13 | PCL-72A_DP/DS-6/3.5 | 77.6 | 95.9 (31.2) | 0.454 (0.124) | 160.0 (5.3) | 0.147 (0.014) |

Table 9 shows the effect of modifier acrylic acid (A) and 2-aminoethyl methacrylate (B) on the synthesis yield, hydrodynamic z-average diameter, polydispersity index (PDI), zeta potential of nanogels made of Dex-PCL-HEMA macromer at 25 and 37° C. The z-average size and zeta potential decreased/increased with increasing the amount of acid/base in the nanogel at 25° C. In contrast, at 37° C. the size decreased with increasing amounts of acid/base. In comparison, the nanogels contain the same mol % of acrylic acid and 2-aminoethyl methacrylate, the nanogels have larger size with positively charged 2-aminoethyl methacrylate than with negatively charged acrylic acid.

TABLE 9
Syntheis yield, hydrodynamic z-average diameter, polydispersity
index (PDI), zeta potential of nanogels.

| | | | | | | Zeta |
|---|---|---|---|---|---|---|
| | | Diameter | | Diameter | | Potential |
| | Yield | nm | PDI | nm | PDI | mV |
| | % | 25° C. | | 37° C. | | 37° C. |
| 10% A | 93.1 | 140.7 ± 12.7 | 0.593 | 96.0 ± 2.1 | 0.166 | −11.5 ± 0.8 |
| 5% A | 88.5 | 121.5 ± 2.2 | 0.528 | 117.1 ± 1.3 | 0.160 | −10.2 ± 0.7 |
| 2% A | 92.5 | 107.9 ± 0.7 | 0.496 | 173.2 ± 0.9 | 0.032 | −7.82 ± 0.1 |
| 0% | 88.9 | 80.3 ± 0.5 | 0.494 | 233.9 ± 0.7 | 0.077 | −0.70 ± 0.3 |
| 2% B | 91.7 | 196.7 ± 6.3 | 0.591 | 265.7 ± 1.8 | 0.115 | 9.3 ± 1.1 |
| 5% B | 92.5 | 136.2 ± 60.8 | 0.468 | 260.9 ± 2.6 | 0.109 | 26.5 ± 0.5 |
| 10% B | 89.7 | 108.3 ± 27.7 | 0.551 | 221.9 ± 8.3 | 0.153 | 30.20 ± 0.3 |

### Example 6. In Vitro Insulin Release Measurements

Insulin release from the thermoresponsive nanogels was investigated in vitro by using a dialysis method (n=4) in which 1 mL of the insulin loaded (if not stated otherwise 30 mg/mL) nanogel was put in a preconditioned 1 mL dialysis device (Float-A-Lyzer® G2 MWCO: 50 kD, Spectrum Laboratories™ Inc.) and the whole dialysis device was immersed in the release media (20 mL, 0.05% NaN3 in pH 7.4 PBS kept in a 50 mL centrifuge tube). The whole set-up was kept in an incubator-shaker (MAXQ 8000, Thermo Fisher Scientific™) maintained 37° C. and shaken at 50 rpm. Then, at a predefined time interval (4 h; 1, 2, 3, 5, 7, 9, 12, 15, 18, 21 days; afterwards weekly) the dialysis device was transferred to a new release medium maintained at 37° C. and the amount of insulin released at each time point was quantitated by UPLC as described herein. As a control, Insulin solution (1 mg/mL) was kept in the dialysis bag and the release of insulin from the solution was similarly determined.

A sensitive stability-indicating UPLC method was developed and a calibration curve was constructed (AUC=11982C−11557) for the quantitation of the released insulin. The method was linear in the range of 2.5 to 100 μg/mL (R²=0.9996) and selective, with all the precursor compounds were eluted before the insulin peak. During the release experiment, to minimize degradation of the released insulin and to simulate the sink condition better, fresh release media was replaced after each time point.

Insulin release from 15% insulin-loaded nanogels synthesized using PLA and PCL based macromers with different DP and DS values at different monomer to macromer ratio using UV polymerization (see Table above) was investigated.

### Example 7. Effect of Monomer to Macromer Ratio on Insulin Release

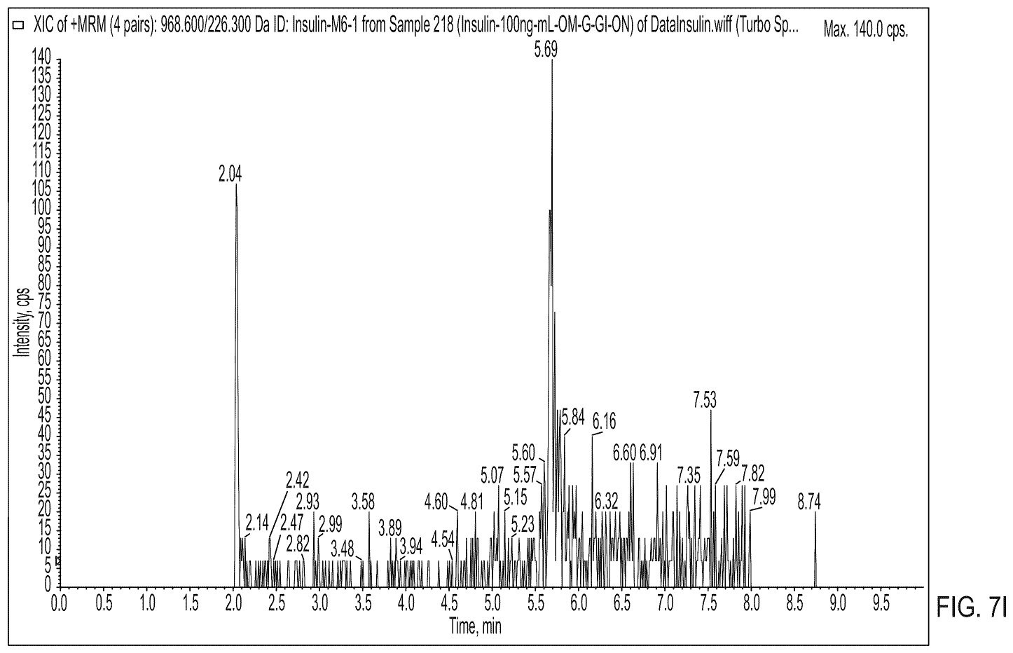

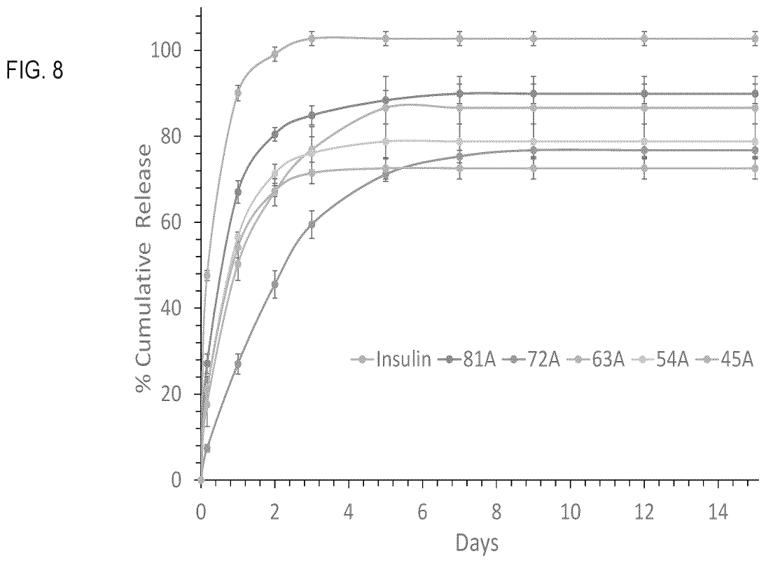

The monomer to macromer ratio had a significant effect on the rate of insulin release; a higher release rate was obtained with 81A nanogels, which can be attributed to the lower degree of crosslinking. A slower insulin release was obtained with the 72A nanogel. Drug release from 63A, 54A, and 45A was not that different (see FIG. 8). 72A nanogel was further used to evaluate the effect of other factors on insulin release.

### Example 8. Effect of Initiator Concentration on Insulin Release

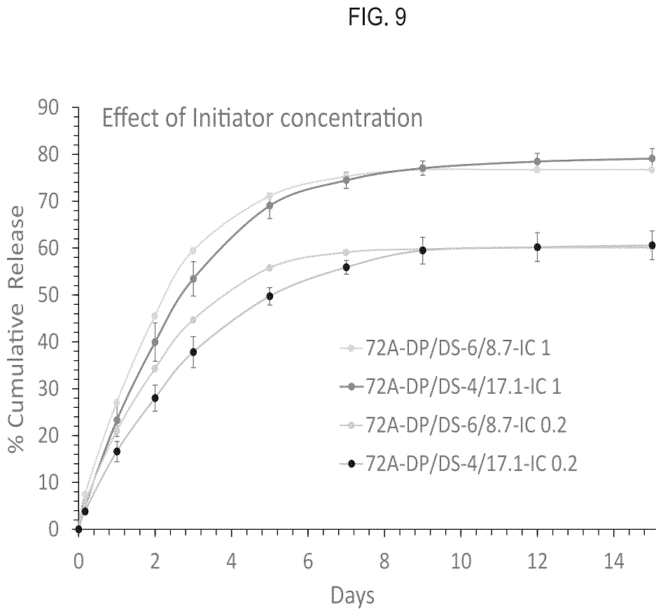

The initiator concentration has a significant effect on insulin release kinetics (see FIG. 9) with the extent of insulin release higher at high initiator concentration, but a similar rate of insulin release.

### Example 9. Effect of DP and DS on Insulin Release

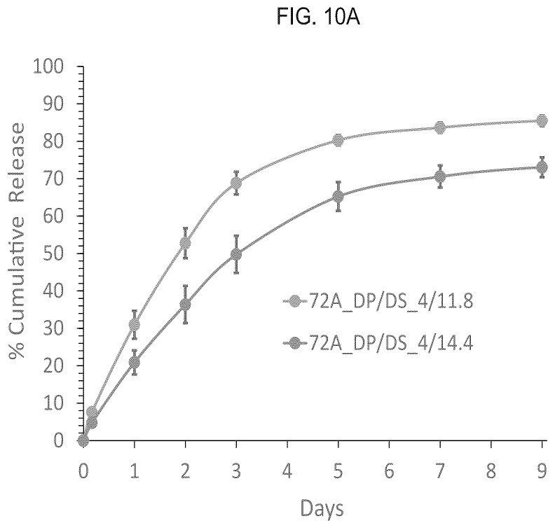

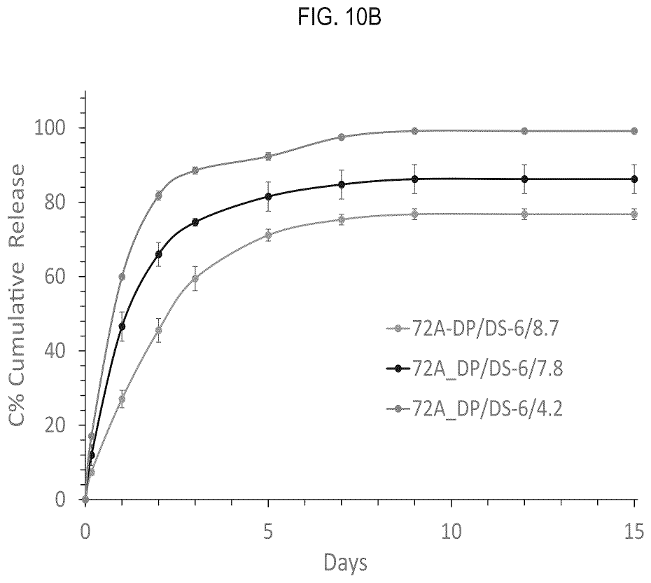

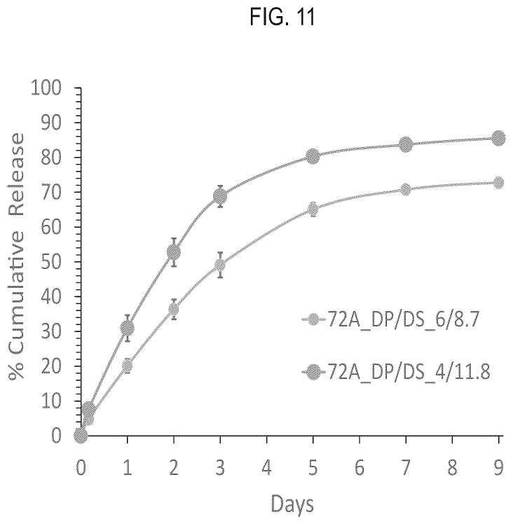

The higher the degree of substitution, the slower was insulin release (see FIG. 10A and FIG. 10B). This is expected because higher DS resulted in increased degree of crosslinking and slower insulin release. However, DP showed no considerable effect on insulin release. Generally, the longer the degree of polymerization, the less the hydrophilicity and the slower the degradation and release of the nanogels in aqueous medium, however this can be nullified by the decrease of DS, which increase the rate of nanogel degradation and rate of insulin release. See FIG. 11, for which release was investigated at 15 mg/mL insulin.

### Example 10. Effect of Macromer Type on Insulin Release

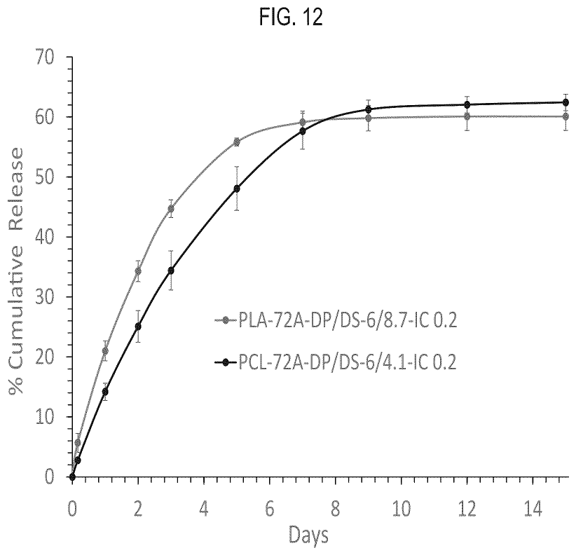

The effect of macromer type was also investigated and insulin release from PCL based nanogels was slower than PLA based nanogels (FIG. 12), which might be attributed to the hydrophobic nature of PLA. The PCL based macromer was even synthesized using macromer with lower DS values indicating the difference would be even higher if PCL macromer with high DS value was used.

### Example 11. Effect of Bulk and Surface Modifiers with Different Charges and Zinc on Insulin Release

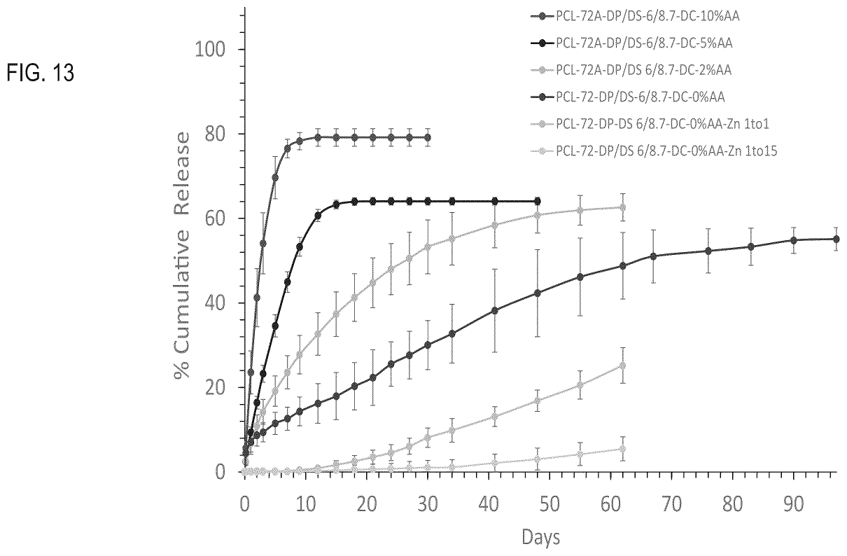

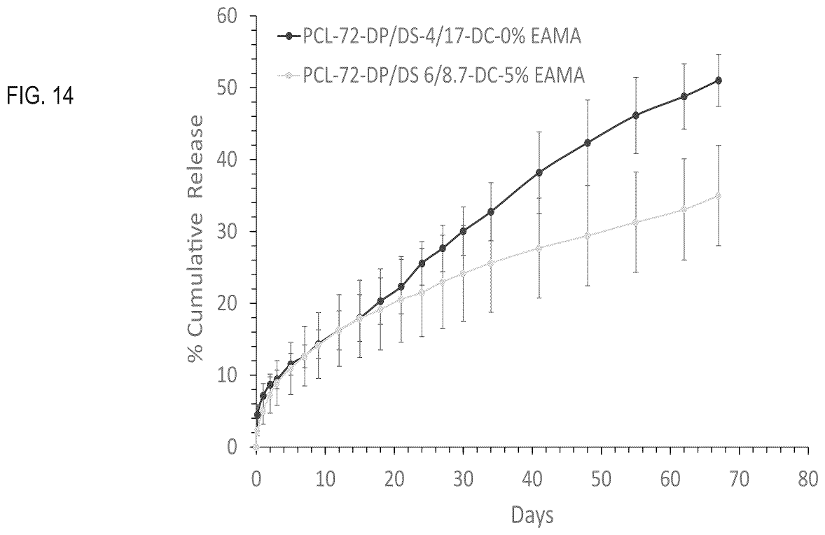

Unlike the effect of monomer to crosslinker ratio, the type and DP and DS of the macromer, insulin release from nanogels were significantly influenced by surface and bulk modifiers. Acrylic acid was used as nanogel modifier nanogels surface charge and membrane permeability but is also had significant effect on insulin release. Generally, using a smaller percentage of acrylic acid with respect to NIPAAm significantly enhanced insulin release from the nanogels and insulin release could be controlled from 9 days to 90 days, by simply decreasing the mole percentage of acrylic acid from 10% to 0% (see FIG. 13). This is particularly true that the isoelectric point of insulin is around 5.3 and at the pH where insulin release was investigated (7.2) insulin is negatively charged. Therefore, the negative charge in the nanogel repels the insulin and enhances its release. Contrarily, the positively charged 2-aminoethyl methacrylate significantly slows down insulin release even though the nanogels containing 2-aminoethyl methacrylate have higher DP and DS (which should slow down the release) than the neutral nanogels without charges (see FIG. 14). The error bars stand for standard error.

Zinc has also been commonly used in different insulin formulations for prolonged insulin action. Particularly it initiates insulin aggregation and slows down its release. Similarly, in our case, addition zinc sulfate during nanogel synthesis considerably slowed down insulin release (see FIG. 13). In addition, its effect was proportional to its concentration. Taken together, insulin release from nanogels is effectively controlled by varying the concentration of acrylic acid with respect to NIPAAm. Zinc sulfate considerably slowed down insulin release from nanogels with effect proportional to the amount of zinc used. 2 amino-allyl methacrylate slowed down insulin release.

### Example 12. Toxicity Study

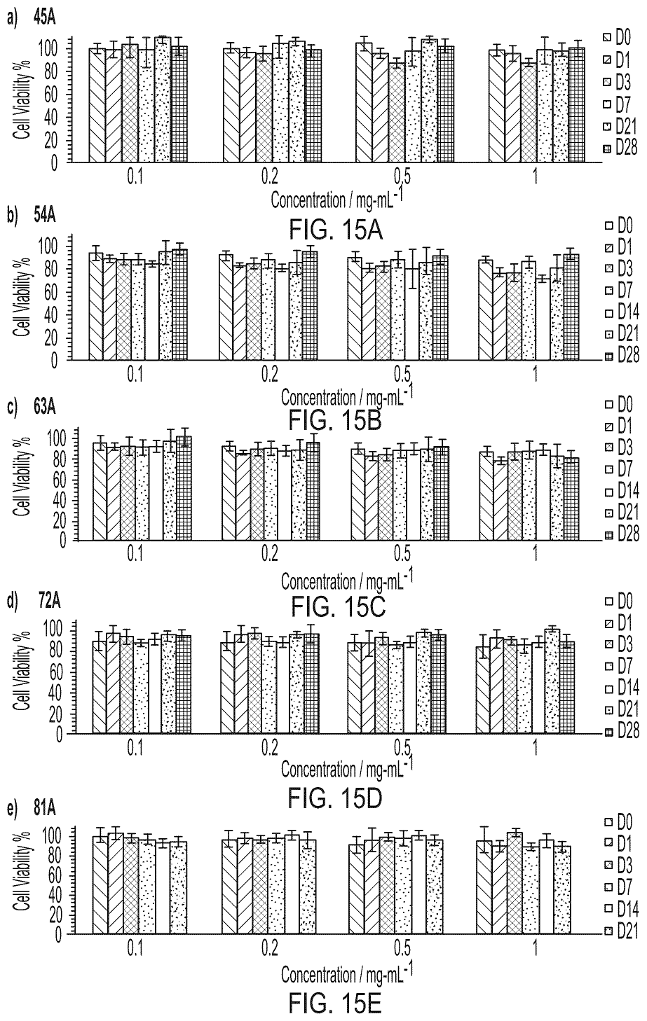

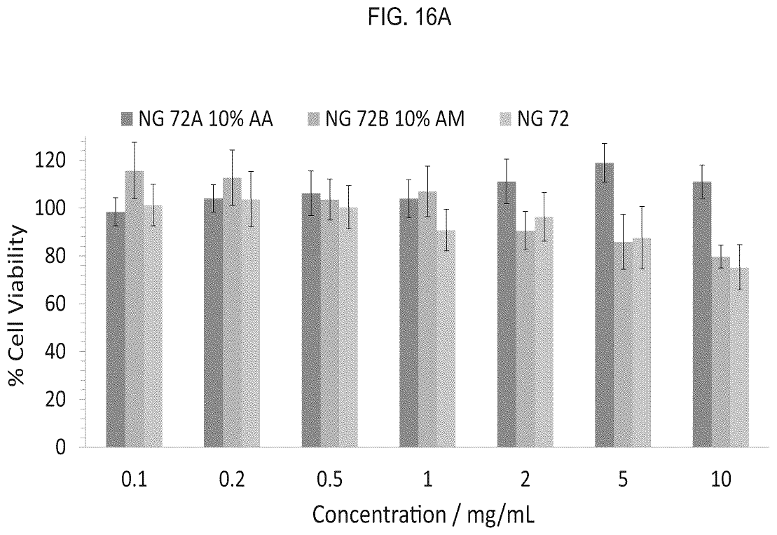

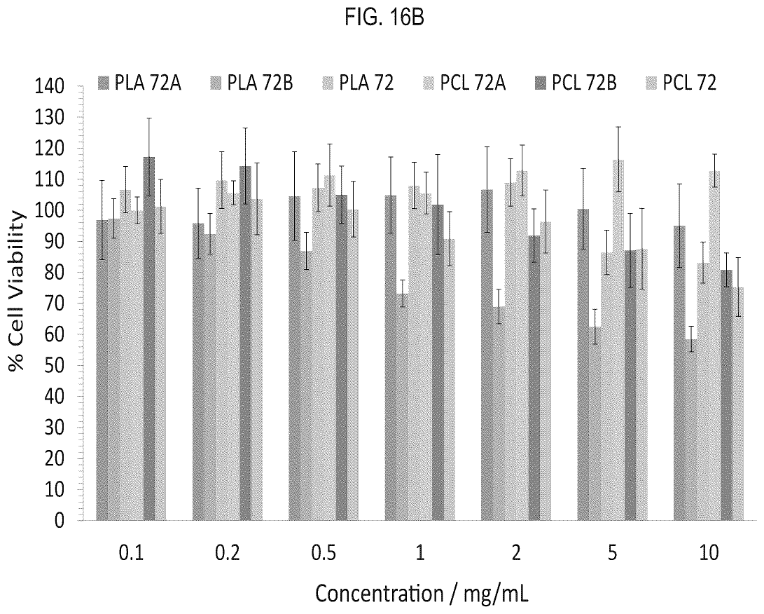

The nanogels were tested for cytoxicity as well. All the nanogels were not cytotoxic to the ARPE-19 cells at up to 5 mg-mL-1. The nanogels were tested for cytoxicity to human adult (ARPE-19) and fetal retinal pigment epithelial (hfRPE) cells at nanogel concentrations of 0, 0.1, 0.2, 0.5, 1, 5, and 10 mg/mL using thiazolyl blue tetrazolium bromide (MTT) assay after 72 hours of incubation at 37° C. and cell seeding density 50,000 cells/cm² in 96 well plates. The toxicity of nanogels synthesized at different monomer to macromer ratios (81A, 72A, 63A, 54A and 45A) and their degradation products, obtained by incubating the nanogels in PBS for up to 4 weeks, was assessed in RPE cells (FIG. 15) by MTT assay. All of the nanogels were safe at a concentration of 1 mg/mL. In addition, the effect of the type of macromer and modifier on insulin toxicity was investigated using MTT assay. Generally, PCL based, negatively charged nanogels showed a relatively better safety profile than PLA based positively charged nanogels (FIG. 16). Taken together, the nanogels were not cytotoxic to the ARPE-19 cells in all tests with cell viability more than 90% at concentration up to 2 mg/mL except for the nanogels made of PLA-containing macromer and positively charged 2-aminoethyl methacrylate. The nanogels made of PCL-containing macromer and negatively charged acrylic acid are not cytotoxic at concentration up to 10 mg/mL. FIG. 15 shows the percent cell viability after an MTT assay of RPE cells exposed to varying concentrations of nanogels synthesized at different monomer to crosslinker ratio. FIG. 16A and FIG. 16B show the percent cell viability after an MTT assay of RPE cells exposed to varying concentrations of nanogels synthesized using PLA and PCL based macromers and using acrylic acid (A) or 2 amino ethyl methacrylate (B) as nanogel bulk and surface modifiers.

### Example 13. Fluorescent Labeling of Nanogels

5-(4,6-Dichlorotriazinyl) Aminofluorescein (5-DTAF) labeled nanogels were prepared by dropwise addition of 3.3 mg 5-DTAF dissolved in 0.2 mL DMSO into 2 mL of 50 mg/mL blank nanogel dispersion in sodium carbonate buffer (0.1 M, pH 9), while stirring. The reaction was carried out at 4° C. overnight. 5-(and-6)-Carboxytetramethylrhodamine (5 (6)-TAMRA) labeled nanogels were prepared by Steglich Esterification. Briefly, 5.56 mg of 5 (6)-TAMRA (1 eq.), 4.3 mg DCC (1.1 eq.) and 9.8 mg 4-dimethylaminopyridine (DMAP)(5 eq.) were dissolved in dichloromethane and stirred at room temperature in the dark for 6 hours. Then the mixture was added dropwise, while stirring, to 185.8 mg nanogel dissolved in dichloromethane and was further stirred at room temperature in the dark overnight. Finally, the nanogels were dialyzed using a 1 KDa MWCO dialysis membrane against DI water for 24 hours, changing the dialysis media every 8 hours, and then lyophilized. The nanogel-dye conjugations were carried out under mild conditions to minimize degradation of the labile lactide bond of the nanogels.

### Example 14. In Vitro Permeability of Nanogels

FITC-dextran (4 kDa) or DTAF labeled nanogels at 1 mg-mL-1 were used for permeability studies. Transwells with 0.45 um pore size were used for the in vitro study and Valia-Chien diffusion cells were used for the ex vivo study. All the permeability studies were done over 4 hours.

The studies were performed as follows. ARPE-19 cells were seeded at a density of 50,000 cells/well on transwell inserts and cultured in DMEM-F12 medium containing 10% FBS and 1% penicillin-streptomycin (10,000 U/mL) at 37° C. with 95% humidity and 5% CO₂. The medium in the wells was changed every other day until maximum TEER value was obtained. 5-DTAF labeled nanogels made of DEX-PCL-HEMA with various charges were suspended in cell culture medium at concentration of 1 mg·mL-1. Negative and positive controls were media without nanogels and FITC-labeled dextran with 4 kDa molecular weight. Transport experiments were conducted in the apical to basal direction at 37° C. for 4 hours. At selected time points 5, 15, 30, 45, 60 minutes and every 30 minutes thereafter, 50 μL medium was taken from the basolateral chamber and replaced with 50 μL of fresh medium. Medium (50 μL) was taken from the apical chamber at the 4 hour time point as well. Aliquots were quantitated using Cytation™ 5 (BioTek, Winooski, VT) at excitation and emission wavelengths of 485 nm and 528 nm, respectively. The permeability (P₀) of the nanogels across the monolayer was calculated by the following formula:

$$Permability Coefficient ( {P}_{0} ) = \frac{Flux ( J )}{Donor Concentration} = \frac{( {F}_{r} / Δ i ) / {A}_{d}}{{F}_{d} / {V}_{d}}$$

where P₀, F_{d}, V_{d}, F_{τ} and A_{d} are the permeability coefficient, the basolateral fluorescence of the solute over Δt time, the fluid volume of the basolateral chamber, the apical fluorescence of the solute, and the surface area of the filter, respectively.

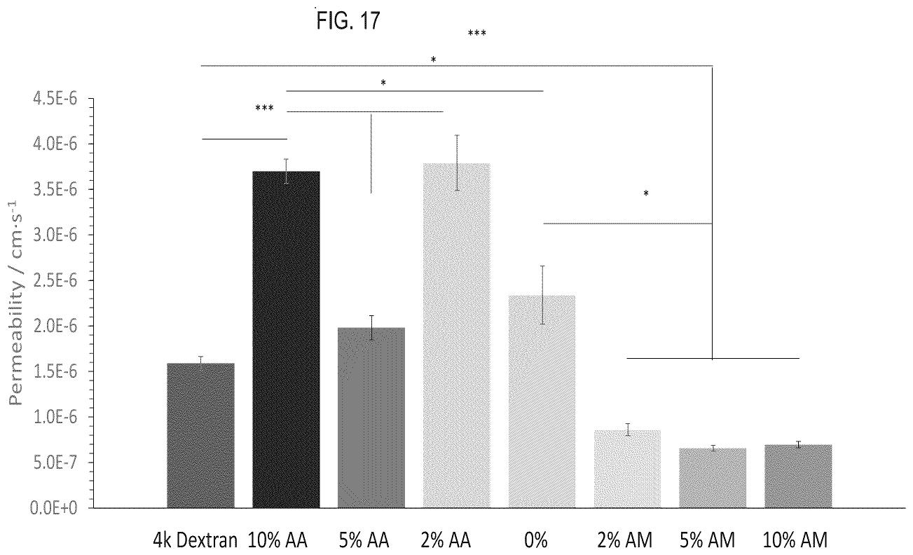

FIG. 17 shows that the permeability across the ARPE-10 cell membrane of neutral and negatively charged nanogels was higher but that of positively charged nanogels was lower than the 4 kDa dextran control. The permeability of negatively charged nanogels with 2 and 10 mol % AA was higher than that of the neutral nanogels and the nanogels with 5 mol % AA. The permeability results correlate well with the cellular uptake of the nanogels by ARPE-19 cells within 4 hours. The greater the amount of nanogels taken up the cells, the more permeable are the nanogels. Due to the binding interaction of the positively charged nanogels with the negatively charged cell membrane, the uptake and permeability of the nanogels were lower than the neutral nanogels. The nanogel particle size increases with increase the charge amount and the aggregation of the nanogels increase with decreasing the charge amount and increasing temperature from 25 to 37° C. Due to the balance of the size and charge, the nanogels containing 5 mol % AA had lower cellular uptake and permeability than the nanogels containing 2 and 10 mol % AA; and there is not much difference in permeability between the positively charged nanogels with 5 and 10 mol % AM.

### Example 15. Ex Vivo Permeability Study

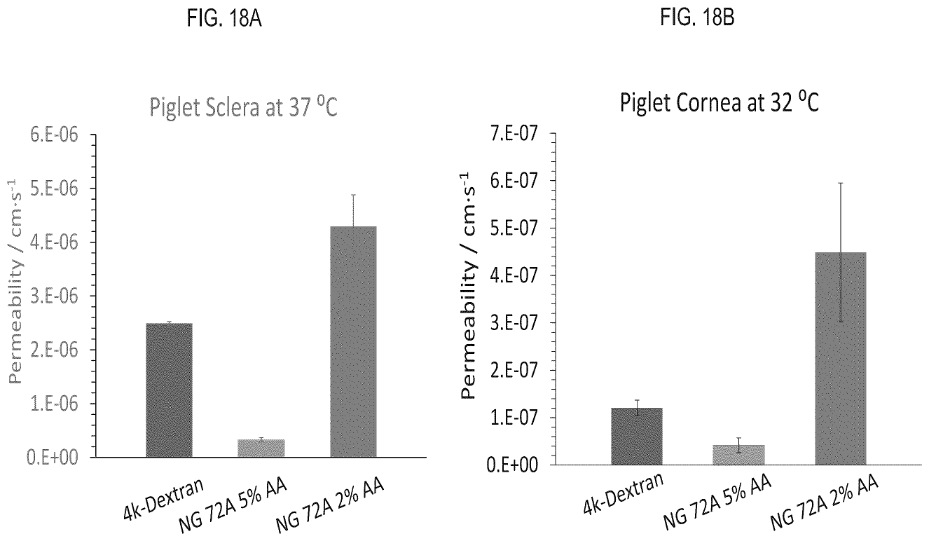

Nanogels made of DEX-PLA-HEMA with 2 mol % acrylic acid was more permeable than those with 5 mol % acrylic acid and 4 kDa dextran control across the ex vivo porcine sclera and cornea tissues. See FIG. 18A and FIG. 18B. The nanogel charge, particle size and composition played important roles on nanogel permeability across ocular barriers.

### Example 16. In Vivo Ocular Nanogel Distribution

5-DTAF labeled 72A nanogel was dispersed in PBS (20 mg/mL) and was injected subconjunctivally (20 μL) to the left eyes of anaesthetized SD rats (n=5) using a 25 gauge Hamilton™ syringe and needle. PBS was injected to the right eyes of the rats to be used as sham controls. After 1 or 7 days of injection, the rats were euthanized, their eyes were inoculated, and the different eye tissues (aqueous humor (AH), vitreous humor (VH), cornea, lens, Iris/ciliary body (CB), sclera, choroid and retina) were collected in a 2 mL flat bottom Eppendorf™ microcentrifuge tube and were kept at −80° C. until tissue homogenization and nanogel extraction. Before homogenization, the tough tissues (lens, cornea and sclera) were ground by a CryoGrinder™ (OPS Diagnostics LLC) at cryogenic temperatures. During tissue extraction, all the tissues were kept on an ice bath and 230 μL cold PBS was added and the samples were homogenized (except the AH and VH) twice for 15 seconds using a rotor-stator (Bio-Gen PRO200 homogenizer, PRO Scientific Inc.) attached with a 5-mm flat bottom generator probe, at a medium speed, with 10-second intervals between the two homogenization steps. Between homogenization of different tissues, the homogenizing probe was rinsed in DI water, followed by 3 washes in 70% ethanol and a final rinse in DI water, and was wiped and dried.

Prior to the homogenization step, to offset the effect of dilution, sample loss & nanogel tissue binding, a fixed amount of rhodamine (TAMRA) labeled nanogel was added as an internal standard (20 μl, 2 mg/mL TAMRA labeled nanogel dispersed in PBS). The homogenate was then centrifuged for 5 minutes at 5000 rpm and the supernatant was put in a 96-well plate and the fluorescent intensity was read at excitation/emission wavelength of 492/519 nm (DTAF) and 541/565 (TAMRA) using a fluorescent reader (Cytation® 5 Cell Imaging Multi-Mode Reader, BioTek Instruments Inc.). A calibration curve of the DTAF label was constructed at a concentration of 5 to 100 μg/mL after adding 160 μg/mL of TAMRA-labeled nanogel as an internal standard.

### Example 17. In Vivo Nanogel Ocular Distribution after Subconjunctival Injection

An efficient tissue homogenization and nanogel extraction method was developed for the quantitation of nanogels in different ocular tissues. The nanogel was labeled with the fluorescent dye 5-DTAF (absorption/emission maxima of ˜492/516 nm) to be able to track its distribution in the tissues. Nanogel recovery from the different ocular tissue homogenates spiked with 5-DTAF-labeled nanogel ranged from 76.6% to 88.2% (see Table 10, below). The nanogel recovery after tissue spiking before homogenization step was lower (49.6%-77.7%). To improve the recovery and reproducibility of the results, 5-(6) TAMRA-(absorption/emission maxima of ˜557/583 nm) labeled nanogel was used as an internal standard. 5-(6) TAMRA-labeled nanogel was chosen over the free dye assuming that the nanogel undergoes similar degree of tissue binding and precipitation. In addition, the two dye-labeled nanogels had different absorption and excitation wavelengths and minimal quenching and interference during measurements occurs. Protein precipitation using organic solvent also significantly reduced the nanogel recovery, and the loss was higher with acetonitrile.

TABLE 10
Percentage nanogel recovery from spiked ocular samples
before and after homogenization in PBS and protein
precipitation in different solvents steps.

| | Nanogel recovery | | | | | |
|---|---|---|---|---|---|---|
| | (%): tissue spike | | Nanogel recovery (%): tissue spiked | | | |
| | with nanogel after | | with nanogel before homogenization | | | |
| | homogenization | | | | | Acetonitrile: |
| Tissue/ | In | Methanol- | In | Methanol- | Acetonitrile- | methanol |
| Sample | PBS | extracted | PBS | extracted | extracted | (1:1) -extracted |
| AH | 87.7 | 81.8 | 71.9 | 85.4 | 95.2 | 77.9 |
| VH | 84.5 | 82.2 | 69.5 | 66.7 | 62.1 | 67.7 |
| Lens | 88.2 | 48.1 | 49.6 | 26.9 | 7.2 | 16.5 |
| Cornea | 81.8 | 79.9 | 77.7 | 68.8 | 72.5 | 71.5 |
| Sclera | 85.1 | 68.2 | 57.5 | 53.8 | 39.6 | 60.2 |
| CB/Iris | 76.6 | 77.8 | 67.9 | 69.3 | 51.3 | 77.6 |
| Choroid | 84.4 | 81.9 | 66.5 | 68.1 | 33.3 | 66.6 |
| Retina | 86.7 | 85.9 | 67.7 | 66.6 | 20.1 | 66.1 |

However, the auto-fluorescence from the non-protein precipitated samples at the excitation wavelengths of the dyes was not significant. Consequently, a protein precipitation step was not considered in the tissue homogenization and nanogel extraction process. Accordingly, a calibration curve of 5-DTAF-labeled nanogels in the range of 0.05 μg/mL to 200 μg/mL was obtained by using 160 g/mL of 5-(6) TAMRA labeled nanogel as an internal standard. The calibration curve was linear (R²=0.9998) in the range of 0.05 to 75 μg/mL, with a calibration equation fluorescence ratio=0.0821C+0.0184. The nanogel recovery in AH, VH, Retina, CB/Iris, choroid, lens, cornea, and sclera, after tissue spiking and using 5-(6) TAMRA-labeled nanogel as an internal standard, was 97.4%, 99.0%, 89.8%, 102.1%, 99.3%, 78.4%, 91.9%, 91.1%, respectively.

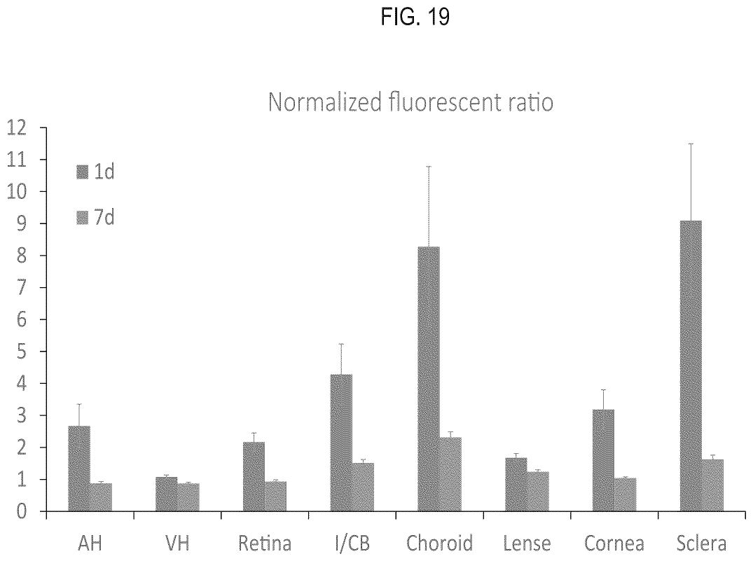

Accordingly, the ocular distribution of the 5-DTAF labeled nanogel in the different eyes were determined after 1 and 7 days of subconjunctival injection and with respect to the control group (results are normalized by the control group) a significant amount of nanogel was recovered in the tissues after 1 day (see FIG. 19). After 7 days, significant amount of nanogel was still recovered in the sclera and choroid. FIG. 19 is a bar graph showing fluorescent intensities of nanogels in different ocular tissues obtained after 1 and 7 days of subconjunctival injection of nanogels to the rat's eye. The values are normalized with the florescent ratio of the control group and values greater than 1 indicate nanogel distribution into the eye.

### Example 18. In Vivo Insulin Pharmacokinetics and Ocular Distribution Study

Insulin-loaded 72A nanogels containing 10% acrylic acid as bulk and surface modifier in PBS (20 mg/mL; equivalent to 3 mg/mL insulin) were injected subconjunctivally (20 μL) to the left eye of anaesthetized SD rats (n=5) using a 25 gauge Hamilton™ needle. The right eyes were injected with PBS to serve as sham controls. After 1, 3 and 7 days of injection, the rats were euthanized, their eyes were inoculated, and the aqueous humor (AH), vitreous humor (VH), cornea, lens, Iris/ciliary body (CB), sclera, choroid and retina were collected in a weighted 2 mL flat bottom Eppendorf™ microcentrifuge tube and were kept at −80° C. until tissue homogenization and insulin extraction.

Similarly, another group of rats (n=5) were injected with an equivalent amount of insulin in PBS. Before tissue homogenization, the tough tissues (lens, cornea and sclera) were ground by a CryoGrinder™ System (OPS Diagnostics LLC) at cryogenic temperatures. During the tissue homogenization steps, all the tissues were kept on an ice bath and 180 μL cold PBS was added and the samples were homogenized (except AH and VH) twice for 15 seconds using a rotor-stator (Bio-Gen™ PRO200 homogenizer, PRO Scientific Inc.) attached with a 5-mm flat bottom generator probe, at a medium speed, with 10 second intervals between the two homogenization steps. Between samples the homogenizing probe was rinsed in DI water, followed by 3 washes in 70% ethanol and a final rinse in DI water, and was wiped and dried.

Prior to the homogenization step, 20 μL 1 mg/mL bovine insulin was added as an internal standard. The homogenate was then kept for 30 minutes and centrifuged for 5 minutes at 6000 rpm. To the supernatant, 200 μL 2% acetic acid in acetonitrile was added and mixed and centrifuged for 5 minutes at 6000 rpm. The obtained supernatant was then dried in a CentriVap™ and the dried sample was reconstituted in 50 μL 1% acetic acid in PBS and was kept for 30 minutes with frequent vortexing. Then the insulin in the desolate was extracted using a solid phase extraction column and the insulin trapped in the column was extracted in 20 μL solvent mixture (acetonitrile: isopropanol: water: HAC 50:24:24:2 v/v/v/v) and quantitated using LC-MS. During solid phase extraction, the column was first activated in 2% HAC in 60% CAN, equilibrated in 1% HAC in water and after extraction the column was washed with 1% HAC. Calibration curve was constructed using 200 μL rat plasma as a surrogate matrix.

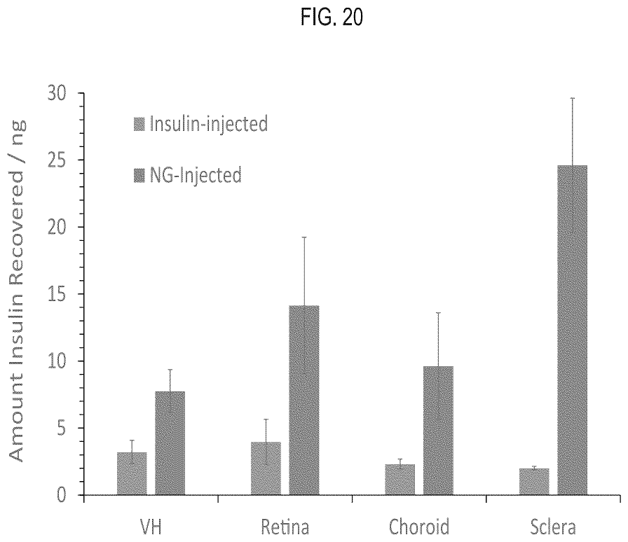

One day after injection, a significant amount of insulin was recovered in different ocular tissues of both group of rats injected with insulin and nanogel, with a significantly higher amount of nanogel recovered in the nanogel injected group (FIG. 20). However, after 3 and 7 days, no appreciable amount of the insulin was detected in the tissues. FIG. 20 shows the amount of human insulin recovered in the different eye tissues of SD rats 1 day after injection of insulin and insulin loaded nanogel (NG).

### Example 19: Loading and Sustained Release of Bevacizumab for Macular Degeneration

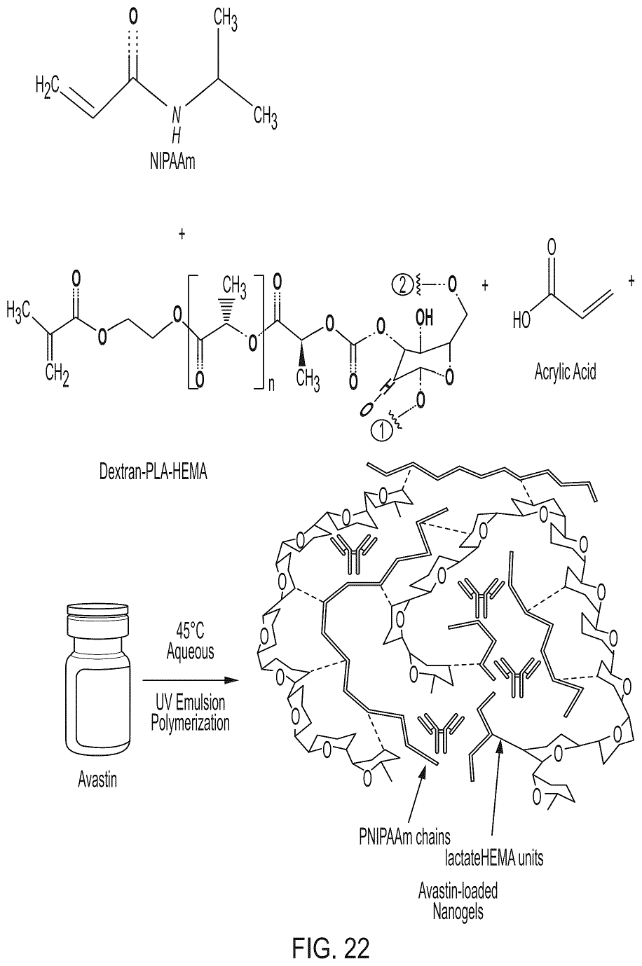

The current standard of care treatment for age-related macular degeneration (AMD) requires monthly intravitreal injections of anti-VEGF therapy Bevacizumab (Avastin®, Genentech). This onerous therapy often leads to low patient compliance and complications including increased ocular pressure and retinal detachment. Here, we show that the inventive biocompatible nanogel system can load and sustain release intact Bevacizumab for 3 months. See FIG. 21. Nanogels containing N-Isopropylacrylamide (NIPAAm) and dextran grafted oligolactate-(2-hydroxyetheyl methacrylate) (Dex-PLA-HEMA, DP=6 and DS=8.7) macromer at weight ratio 7:2, and acrylic acid (AA) at 0, 2, 5, 7 or 10 mol % with respect to NIPAAm were synthesized by UV emulsion polymerization using Irgacure® 2959 as an initiator. During synthesis, the monomer and macromer were dissolved in water (10 mg/mL), and degassed for 10 minutes using N₂ gas. The solution was exposed to UV light (1 W/cm²) (EXFO, Inc., Richardson, TX, USA) under stirring at 300 rpm and 45° C. for 15 minutes. In the case of Avastin-loaded nanogels, 1 to 37.5 wt % Avastin was added to the reaction mixture before the UV polymerization. FIG. 22 illustrates the Avastin-loaded nanogel synthesis process.

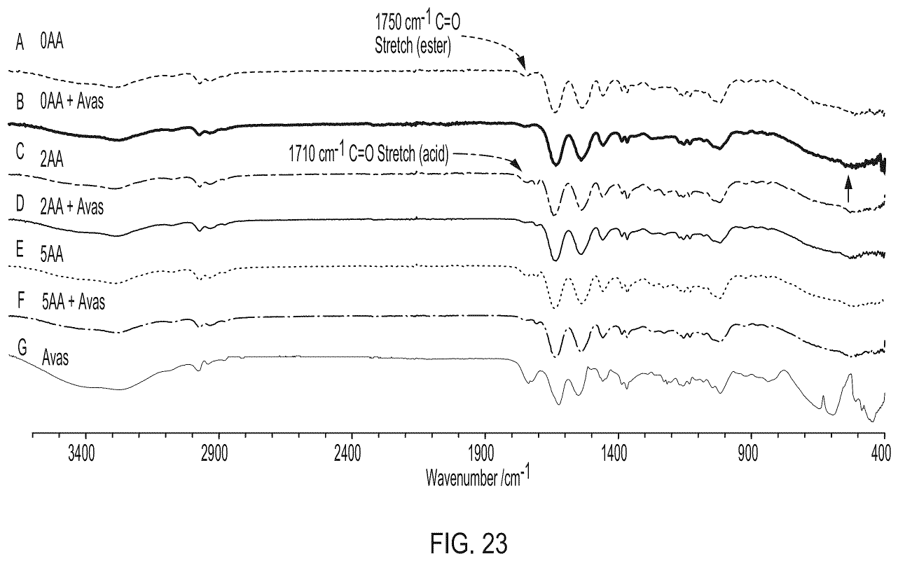

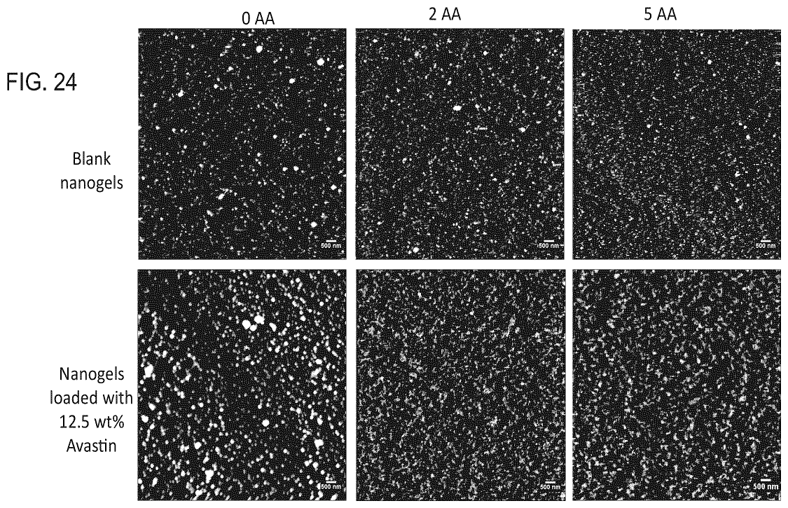

Bevacizumab encapsulation inside the nanogel was evaluated by ATR-FTIR. The FTIR spectra of nanogels containing different amount of acrylic Acid and Avastin show that the peak intensity at 1710 cm-1 corresponding to the C═O stretching of acid increases with increasing amounts of acrylic acid from 0 to 2 to 5 mol %. This indicates the success of incorporation of acrylic acid in the nanogels during the synthesis. The successful loading of Avastin into the nanogels can be observed by increases in the intensity of the broad peak in 800-400 cm-1 region compared to their non Avastin loaded counter parts. See FIG. 23. The size and morphology of the nanogels in water at room temperature were measured and analyzed using dynamic light scattering (DLS) and atomic force microscopy with quantitative imaging mode (AFM QI Mode). See FIG. 24.

In Table 11, below, both the DLS and AFM results show that the particle sizes of the blank nanogels decrease with increasing the AA amount from 0 to 5 wt %. See also FIG. 24. Without wishing to be bound by theory, the reason for this phenomenon is probably that the nanogels aggregated at 37° C. and the repulsive force between the negative charges of AA reduces the aggregation of the nanogels more with increasing AA. The increase of the zeta potential with increasing the AA amount supports this explanation. Table 11 also shows that the incorporation of Avastin in the nanogels increases the size (DLS and AFM data) of the nanogels and makes the nanogels less negatively charged (zeta potential data). In Table 11, the nanogels contained 0, 2 or 5 mol % acrylic acid (AA) with respect to N-isopropylacrylamide (NIPAAm) loaded with and without 12.5 wt % Avastin in water at 37° C.

TABLE 11
Hydrodynamic size measured by DLS, polydispersity index,
zeta potentials and size measured by AFM of nanogels.

| | Hydrodynamic | | | AFM |
|---|---|---|---|---|
| | Diameter | | Zeta | Diameter in |
| | in Water /nm | PDI | Potential | Water /nm |
| 0 AA | 128.37 ± 3.81 | 0.05 ± 0.02 | −6.34 ± 0.99 | 51.81 ± 9.01 |
| 0 AA | 135.25 ± 1.96 | 0.08 ± 0.06 | 0.53 ± 0.14 | 85.20 ± 18.27 |
| Avas | | | | |
| 2 AA | 101.83 ± 2.89 | 0.13 ± 0.02 | −10.26 ± 1.27 | 41.64 ± 7.85 |
| 2 AA | 114.87 ± 3.15 | 0.07 ± 0.01 | −0.10 ± 0.84 | 46.25 ± 7.93 |
| Avas | | | | |
| 5 AA | 75.16 ± 1.61 | 0.17 ± 0.01 | −15.44 ± 0.48 | 28.83 ± 1.66 |
| 5 AA | 79.72 ± 2.00 | 0.18 ± 0.04 | −6.14 ± 2.26 | 42.02 ± 10.79 |
| Avas | | | | |

### Example 20. In Vitro Avastin Release

Nanogels containing 0, 2 or 5 mol % acrylic acid (AA) loaded with 12.5 or 37.5 wt % Avastin were dispersed in 1 mL PBS (pH 7.4) inside a release device (Float-A-Lyzer® G2, Spectrum Laboratories Inc.) with MWCO 300 kDa at 20 mg/mL. The release device was immersed in 20 mL PBS (pH 7.4) in a 50 mL conical centrifuge tube, and the release was carried out in an incubator shaker at 70 rpm and 37° C. for 100 days. At selected times, the release device was transferred to a new PBS (pH 7.4) release media maintained at 37° C. The amount of Avastin released at each time point was measured using a BCA total protein assay kit (n=4). The fluorescence spectra of released Avastin at late time points were compared to native Avastin® to evaluate the structural integrity of the therapeutic antibody during release.

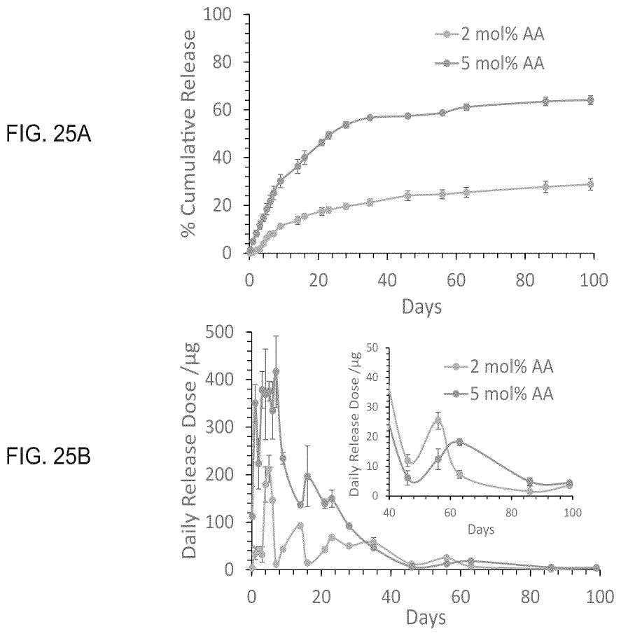

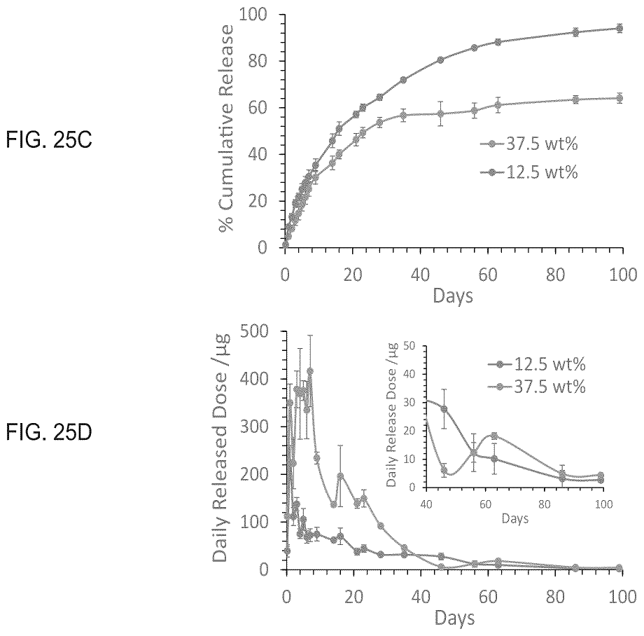

FIG. 25A through FIG. 25D show that the nanogels can sustain the release of Avastin for 100 days, and the release kinetics can be tailored in two ways. First, increasing acrylic acid amount from 2 to 5 mol % increases the Avastin® release rate (see FIG. 25A and FIG. 25B, showing different mol % of acrylic acid). Second, increasing the Avastin® loading amount from 12.5 wt % to 37.5 wt % decreases the Avastin® release rate (FIG. 25C and FIG. 25D, showing different wt % Avastin®). Thus, the release kinetics of the loaded drug depend on the acrylic acid amount and Avastin loading content and can be adjusted thereby.

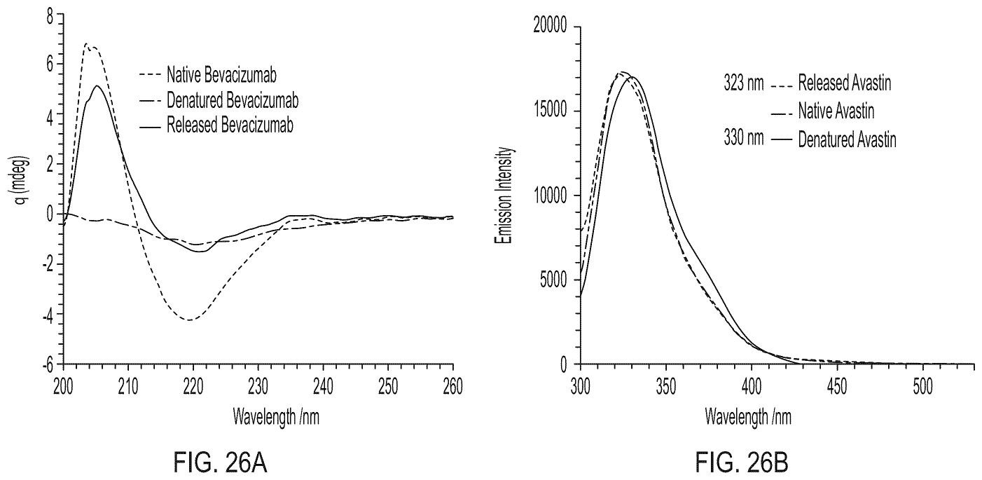

Furthermore, FIG. 26 shows the Avastin released from the nanogels maintains its integrity with its second and tertiary protein structures intact. FIG. 26A and FIG. 26B present far-UV (FIG. 26A) and fluorescence emission (FIG. 26B) scans, respectively, of native, denatured, and released Avastin® from the nanogels. The data demonstrate that both secondary and tertiary protein structures of Avastin® was intact after release from the nanogels. From both graphs, it can be recognized that released Avastin® shows spectral similarity to their native controls and are unlike the denatured controls.

### Example 21. In Vitro Cytotoxicity of Nanogels

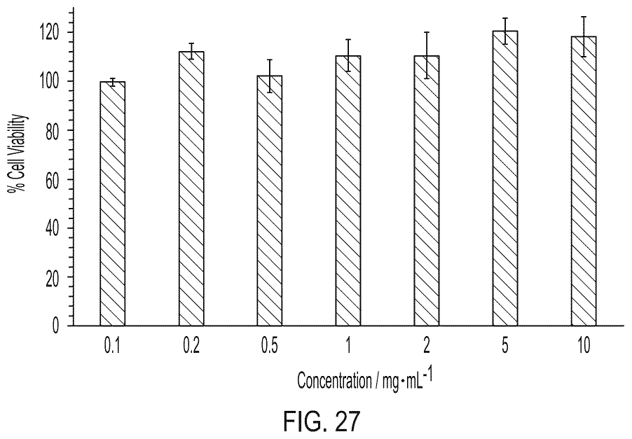

The cytotoxicity of the nanogels to human fetal retinal pigment epithelial (hfRPE) cells was assessed at nanogel concentrations of 0, 0.1, 0.2, 0.5, 1, 5, and 10 mg/mL using a thiazolyl blue tetrazolium bromide (MTT) assay after 72 hours of incubation at 37° C. and cell seeding density 50,000 cells/cm² in 96 well plates. The nanogels were not toxic to hfRPE cells at concentration up to at least 10 mg/mL. FIG. 27.

### Example 22. In Vitro Bioeffect of Avastin-Loaded Nanogels

To test the ability of Avastin®-loaded nanogels to affect VEGF expression, HfRPE cells were seeded in 96-well plates at a density of 100,000 cells/cm². After one day incubation, cell media for all groups was changed to PBS (pH 7.4). Half of all cells were exposed to UV light (1 J/cm) for one hour, and the other half were placed back into the cell incubator. After the 1-hour exposure, PBS (pH 7.4) for all wells were replaced with serum free media (DMEM+pen/strep), then Avastin® at concentration 0.25 mg/mL, and nanogels containing 2 mol % AA with and without 12.5 wt % Avastin® were added into the wells at a concentration of 2 mg/mL. Cells with and without UV treatment were used as controls.

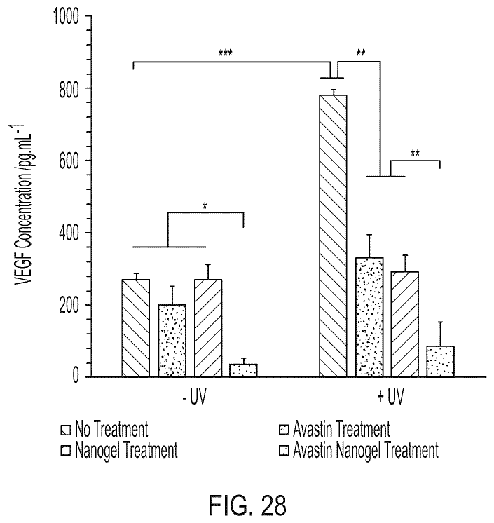

After 24 hours, a VEGF ELISA assay was conducted on all experimental groups. The data in FIG. 28 show that Avastin® and nanogels alone do not change VEGF expression of untreated hfRPE. UV treatment increases the VEGF expression of hfRPE more than 2 times, and treatment of Avastin® or nanogels alone reduces the VEGF expression to the level of the cells without UV treatment. Therefore, treatment with Avastin®-loaded nanogels reduces the VEGF expression in both non-UV- and UV-treated hfRPE cells.

### Example 23. siRNA- and Anti-TNFα-Loaded Nanogels

Nanogels containing siRNA or anti-TNFα were synthesized by UV emulsion polymerization of N-Isopropylacrylamide (NIPAAm), dextran grafted oligocaprolactone-(2-hydroxyetheyl methacrylate) (Dex-PCL-HEMA, DP=6 and DS=4.1) and acrylic acid (AA) with the presence of siRNA or anti-TNFα, respectively. During the synthesis, NIPAAm (77.8 mg), Dex-PCL-HEMA macromer (22.2 mg), AA (5 mol % with respect to NIPAAm) and siRNA (6.65 μg) or anti-TNFα (3 μg) were first dissolved in 3.48 mL RNase free or DI water, respectively, and degassed for 10 minutes using N₂ gas. The initiator Irgacure® 2959 (1 wt %) was added to the precursor solution, and UV light (1 W/cm²; wavelength 320-500 nm; EXFO, Inc.) was shined on the solution to polymerize the monomers and macromer. The polymerization was conducted under stirring at 300 rpm and 45° C. for 15 minutes. After the synthesis, the nanogels were dialyzed in DI water through a dialysis membrane (Spectra/Por® 6 Dialysis Membrane MWCO: 50 kD, Spectrum Laboratories Inc.) for 5 hours, with frequent changes of the dialysis media (every 1 hour). The purified nanogel dispersion was lyophilized to obtain dry nanogels.

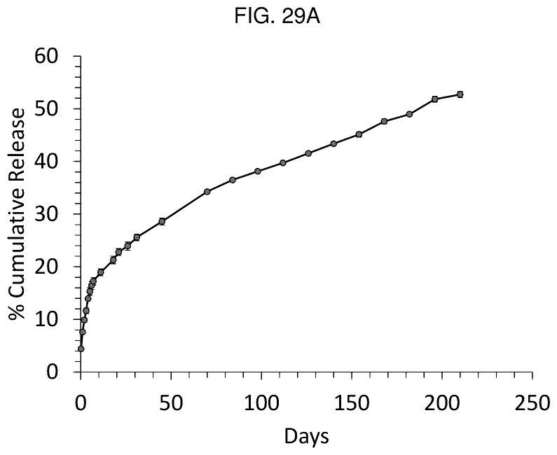

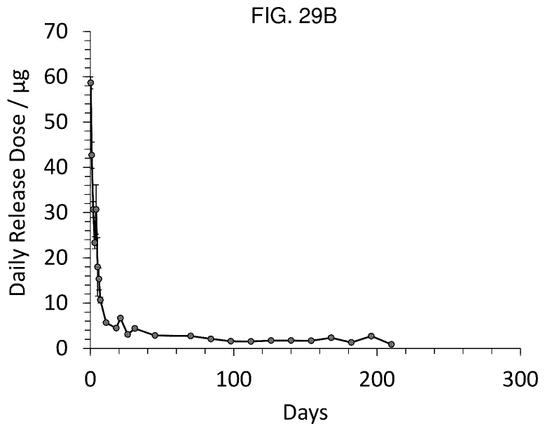

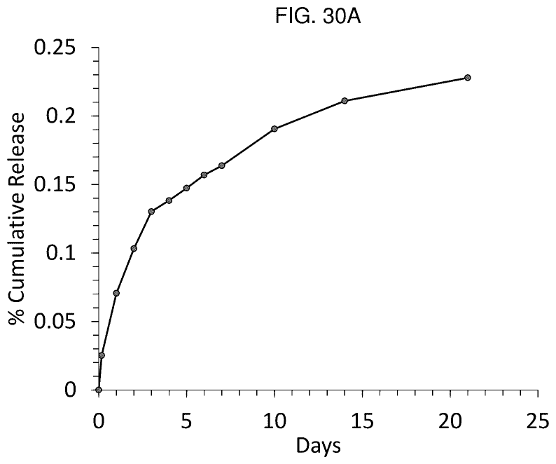

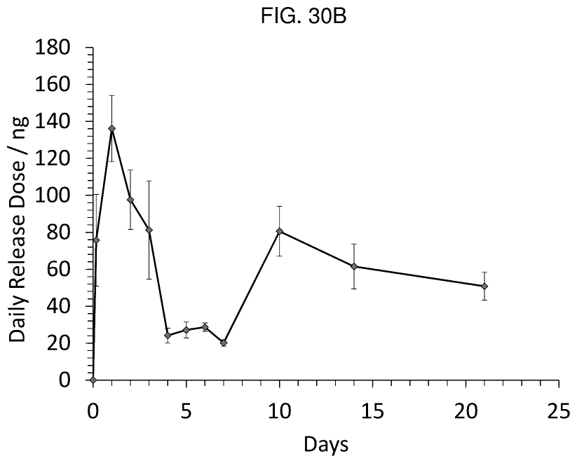

The siRNA or anti-TNFα-loaded nanogels were dispersed in 1 mL PBS (pH 7.4) inside a release device (Float-A-Lyzer® G2, Spectrum Laboratories Inc.) with MWCO 300 kDa at 20 mg/mL. The release device was immersed in 20 mL PBS (pH 7.4) in a 50 mL conical centrifuge tube, and the release was carried out in an incubator shaker at 70 rpm and 37° C. for seven months. At selected times, the release device was transferred to a new PBS (pH 7.4) release media maintained at 37° C. The amounts of siRNA and anti-TNFα released at each time point were determined using Nanodrop® spectrophotometry and ELISA, respectively (n=4). FIG. 29 and FIG. 30 present data that show that the nanogels can sustain the release of siRNA and anti-TNFα for seven months and three weeks, respectively (n=4).

### REFERENCES

All references listed below and throughout the specification are hereby incorporated by reference in their entirety.
1. Sahle F F, Fili C V, Kim S Y, Tang J Z, Hamilton D J and Lowe T L*. Formulation Optimization and Ocular Pharmacokinetics and Toxicity Investigations of Insulin-Loaded Thermoresponsive Biodegradable Nanogels Intended for the Treatment of Diabetic Retinopathy. 2018 PharmSci 360, American Association of Pharmaceutical Scientists (AAPS) Annual Meeting and Exposition, Washington DC, Nov. 6, 2018. Poster.
2. Kim S Y, Sahle F F, Ghonim N and Lowe T L*. Investigation of Surface Charge Effects on Insulin-Loaded Thermoresponsive Biodegradable Nanogels Intended for the Treatment of Diabetic Retinopathy. Formulation and Delivery—Chemical—Drug Delivery—Nanoparticles Track, 2020 PharmSci 360, American Association of Pharmaceutical Scientists (AAPS) Annual Meeting and Exposition, Virtual, Oct. 26-Nov. 5, 2020. Poster.
3. Huang, X. and T. L. Lowe, Biodegradable Thermoresponsive Hydrogels for Aqueous Encapsulation and Controlled Release of Hydrophilic Model Drugs. Biomacromolecules, 2005. 6(4): p. 2131-2139.
4. Imai, H., et al., Subconjunctivally Implanted Hydrogels for Sustained Insulin Release to Reduce Retinal Cell Apoptosis in Diabetic Rats. Investigative Ophthalmology & Visual Science, 2015. 56(13): p. 7839-7846.
5. Misra, G. P., D. R. Janagam, and T. L. Lowe, Effect of Excipients on the Stability of Insulin Lispro. Macromolecular Symposia, 2015. 351(1): p. 46-50.
6. Lowe T L, Kim Y S and Huang X. Multi-functional Polymeric Materials and Their Uses. U.S. Pat. No. 8,545,830, Issued Oct. 1, 2013.

1. A nanogel pharmaceutical composition, comprising:
(a) a biologic medicament; and
(b) a biodegradable nanogel composition comprising
(i) a macromer according to Formula I wherein CLU is a crosslinkable unit, HS is a hydrolysable spacer, and DU is a dextran or polysaccharide unit;
CLU-HS-DU Formula I
(ii) a hydrolyzable crosslinker;
(iii) a monomer; and
(iv) an initiator,
wherein the macromer, the monomer, and the hydrolysable crosslinker are reacted with an initiator to form a biodegradable nanogel.

2. The nanogel pharmaceutical composition of claim 1 wherein the biologic medicament is selected from the group consisting of a protein, a peptide, a nucleic acid, an antibody, a monoclonal antibody, a gene, a nucleic acid (RNA or DNA), a nucleotide, a oligonucleotide, a siRNA, a mRNA, an aptamer, a vaccine, a receptor, an enzyme, a ligand, a hormone, a blood product, a biopolymer, a natural polymer, a biomolecule, a biomacromolecule, a poly(amino acid), a protein kinase, a cytokine, a growth factor, a differentiation factor, a neurotrophic factor, a stem cell factor, a fusion protein, a carbohydrate, a polysaccharide, a lipid, a lipopolysaccharide, a glycosaminoglycan, a steroid, a nutrient, a tumor necrosis factor (TNF) inhibitor, an interleukin (IL) inhibitor, a B-cell inhibitor, or a T-cell inhibitor, or a combination thereof.

3. The nanogel pharmaceutical composition of claim 2 wherein the biologic medicament is selected from the group consisting of a peptide hormone, an antibody, an aptamer, and an siRNA or a combination thereof.

4. The nanogel pharmaceutical composition of claim 1 wherein CLU is a hydrolytically or enzymatically degradable, crosslinked nanogel.

5. The composition of claim 1, wherein the initiator is 2-hydroxy-4′-(2-hydroxyethoxy)-2-methylpropiophenone.

6. The composition of claim 1, wherein the monomer is N-isopropylacrylamide.

7. The nanogel pharmaceutical composition of claim 1 wherein the biologic medicament is released from the nanogel for at least 20 days.

8. The nanogel pharmaceutical composition of claim 1 wherein the biologic medicament is released from the nanogel for at least 30 days.

9. The nanogel pharmaceutical composition of claim 1 wherein the biologic medicament is released from the nanogel for at least 60 days.

10. The nanogel pharmaceutical composition of claim 1 wherein the biologic medicament is released from the nanogel for at least 90 days.

11. The nanogel pharmaceutical composition of claim 1 wherein the biologic medicament is loaded in the nanogels in aqueous solution with at least 50% loading efficiency.

12. The nanogel pharmaceutical composition of claim 1 wherein the biologic medicament is loaded in the nanogels in aqueous solution with at least 80% loading efficiency.

13. The nanogel pharmaceutical composition of claim 1 wherein the biologic medicament is loaded in the nanogels in aqueous solution with at least 95% loading efficiency.

14. The method of treating a subject in need of a biologic medicament, comprising administering to the subject a pharmaceutical composition of claim 1.
